(19) United States
(12) Patent Application Publication
(10) Pub. No.: US 2025/0243220 A1
(43) Pub. Date: Jul. 31, 2025

(54) ATR INHIBITORS

(71) Applicant: APREA THERAPEUTICS, INC., Ambler, PA (US)

(72) Inventors: Joseph VACCA, Telford, PA (US); Dansu LI, Warrington, PA (US); Lanqi JIA, Ambler, PA (US); Oren GILAD, Ambler, PA (US)

(21) Appl. No.: 18/855,713

(22) PCT Filed: Apr. 14, 2023
(86) PCT No.: PCT/US2023/065755

§ 371 Date: Oct. 10, 2024

Related U.S. Application Data
(60) Provisional application No. 63/330,944, filed on Apr. 14, 2022.

(51) Int. Cl. C07D 515/08; A61K 31/4995; A61P 35/00

(52) U.S. Cl. CPC C07D 515/08; A61K 31/4995; A61P 35/00

(57) ABSTRACT

The disclosure provides compounds of Formula (I), or a pharmaceutically acceptable salt thereof, wherein R′-R⁷ and L are defined herein: Also provided are pharmaceutical compositions, comprising one or more compounds described herein and methods of treating cancer in a patient comprising administering to the patient a compound or pharmaceutical composition described herein.

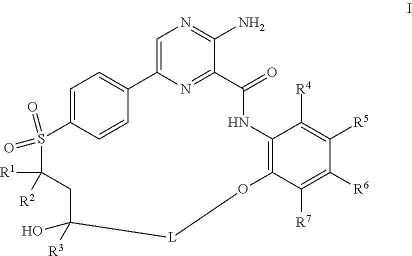

### CROSS-REFERENCE TO RELATED APPLICATIONS

This application claims priority of U.S. Provisional Patent Application No. 63/330,944, filed Apr. 14, 2022, the disclosure of which is incorporated by reference herein.

### TECHNICAL FIELD

The present disclosure relates to ATR inhibitors and methods of use thereof.

### BACKGROUND

Ataxia telengiectasia and rad3-related (ATR) protein kinase is integral to the replication stress response. ATR belongs to a family of kinases, i.e., phosphatidyl inositol 3′ kinase-related kinases (PIKKs), that are involved in the signaling scheme and repair of DNA damage. While other members of this family (ataxia-telangiectasia mutated (ATM) and DNA-dependent protein kinase catalytic subunit (DNA-PKcs)) are required for the repair of double strand breaks (DSBs), ATR is recruited to, and activated by, single strand DNA (ssDNA) generated at stalled replication forks or as an intermediate in the repair of DSBs. Upon replication fork stalling activated ATR phosphorylates the downstream kinase Chk1 resulting in stabilization of the replication fork and inhibition of cell-cycle progression, thus allowing time for resolution of the stress and continued replication. When the ATR-Chk1 pathway is disrupted stalled replication forks collapse into DSBs, thus if unresolved, replication stress can cause genomic instability and negatively impact cell survival. Due to its vital role in replication, loss of ATR is early-embryonic lethal in mice. However, it is important to note that significant suppression of ATR activity (by more than 90%) by mutations in ATR is well tolerated by bone marrow and intestinal epithelium, the tissues that are most sensitive to traditional chemotherapeutics.

ATR inhibition is synthetically lethal in cancers with mutations that cause oncogenic stress or disruption of the DNA damage response (DDR). Genetic changes associated with cancer promote the activation of the replicative stress response and other DNA damage response (DDR) pathways. Such oncogenic stress inducing alterations include K-Ras^{G12D} and H-Ras^{G12V} mutations, and c-Myc amplification. Activation of the DDR by oncogenic stress has been proposed to contribute to selection for mutation, and loss of, p53 and ATM. Mutations in the tumor suppressor p53 are found in ˜50% of all human cancers. Similar mutation frequencies are observed in the oncogene Myc, while significant numbers of cancers also harbor mutations in the Ras family of genes (˜16%) and to a lesser degree the DDR protein ATM. Alterations in these genes cause an increased reliance on the ATR-Chk1 pathway for genome maintenance. Studies have found that ATR inhibition elicits synthetic lethality under each of these cancer associated conditions.

Cancers deficient in components of the homologous recombination pathway, such as those harboring mutations in BRCA1 and BRCA2, are highly sensitive to PARP inhibition. While PARP is required for the repair of single strand breaks (SSBs), preventing their collapse into DSBs, ATR stabilizes replication forks, similarly preventing collapse and formation of DSBs. Loss of PARP and ATR activities therefore both force cells to rely on the DSB repair pathway. It is the inability of BRCA mutant cells to repair DSBs that renders them sensitive to PARP inhibition, it is therefore reasonable to suppose that cells deficient in the DDR, such as those harboring BRCA mutations, would also be sensitive to ATR inhibition.

Compounds that inhibit ATR are needed.

### SUMMARY

In some embodiments, the disclosure provides compounds of Formula (I), or a pharmaceutically acceptable salt thereof, wherein R¹-R⁷ and L are defined herein:

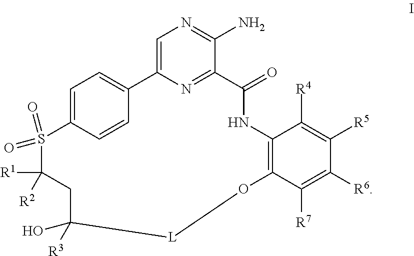

In other embodiments, the disclosure provides pharmaceutical compositions, comprising one or more compounds described herein.

In further embodiments, the disclosure provides methods of treating cancer in a patient comprising administering to the patient a compound or pharmaceutical composition described herein.

### DETAILED DESCRIPTION OF ILLUSTRATIVE EMBODIMENTS

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect.

In the following descriptions of exemplary embodiments of the present invention, all references, including publications, patent applications, and patents, cited herein are incorporated by reference into this application to the same extent as if each reference were individually and specifically indicated to be incorporated by reference and were set forth in its entirety herein.

Conversely, various features of the disclosed compositions and methods that are, for brevity, described in the context of a single aspect, may also be provided separately or in any subcombination.

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

The term “alkyl” refers to a straight- or branched-chain alkyl group having from 1 to 12 carbon atoms (“C₁₋₁₂”), preferably 1 to 6 carbons atoms (“C₁₋₆”), in the chain. Examples of alkyl groups include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and groups that in light of the ordinary skill in the art and the teachings provided herein would be considered equivalent to any one of the foregoing example. Unless stated otherwise, an alkyl group is optionally substituted by one or more of OH, halo, CN, NO₂, C₁₋₆alkoxy, C₃₋₄cycloalkyl, heterocycloalkyl, aryl, or heteroaryl.

“Cycloalkyl” refers to a saturated monocyclic or polycyclic radical that contains carbon and hydrogen. In some embodiments, cycloalkyl includes groups having from 3 to 12 ring atoms (i.e., (C₃₋₁₂)cycloalkyl). Illustrative examples of cycloalkyl groups include, but are not limited to, cyclopropyl, cyclobutyl, cyclopentyl, cyclohexyl, cycloheptyl, or cyclooctyl and the like. Unless stated otherwise, a cycloalkyl group is optionally substituted by one or more of OH, halo, CN, NO₂, C₁₋₆alkyl, C₁₋₆alkoxy, C₃₋₈cycloalkyl, heterocycloalkyl, aryl, or heteroaryl.

“Heterocycloalkyl” refers to a saturated monocyclic or polycyclic radical that contains carbon and hydrogen. In some embodiments, heterocycloalkyl includes groups having from 3 to 12 ring atoms (i.e., (C₃₋₁₂)heterocycloalkyl) and one nitrogen or oxygen atom. Illustrative examples of heterocycloalkyl groups include, but are not limited to azepanyl, azetidinyl, aziridinyl, azocanyl, azolidinyl, dioxanyl, oxetanyl, oxanyl, oxepanyl, oxiranyl, oxocanyl, oxolanyl, piperidinyl, pyranyl, pyrrolidinyl, tetrahydrofuranyl, thianyl, thiepanyl, thietanyl, thiiranyl, thiocanyl, thiolanyl, or the like. In some embodiments, the heterocycloalkyl is pyrrolidinyl, tetrahydrofuranyl, oxetanyl, pyranyl, piperidinyl, or azetidinyl. Unless stated otherwise, a heterocycloalkyl is optionally substituted by one or more of OH, halo, CN, NO₂, C₁₋₆alkyl, C₁₋₆alkoxy, C₃₋₈cycloalkyl, heterocycloalkyl, aryl, or heteroaryl.

“Halo” refer to fluoro, chloro, bromo or iodo. In some embodiments, the halo is fluoro. In other embodiments, the halo is chloro. In further embodiments, the halo is bromo. In yet other embodiments, the halo is iodo.

“Alkoxy” refers to a straight- or branched-chain alkyl group having from 1 to 12 carbon atoms (“C₁₋₁₂”), preferably 1 to 6 carbons atoms (“C₁₋₆”), and one oxygen atom in the chain. Examples of alkyl groups include methyl (OMe), ethyl (OEt), n-propyl (OⁿPr), isopropyl (OⁱPr), butyl (OBu), isobutyl (OⁱBu), sec-butyl (O^{s}Bu), tert-butyl (O^{t}Bu), pentyl (O-pentyl), isopentyl (O-ⁱpentyl), tert-pentyl (O-^{t}pentyl), hexyl (O-hexyl), isohexyl (O-ⁱhexyl), and groups that in light of the ordinary skill in the art and the teachings provided herein would be considered equivalent to any one of the foregoing example. Unless stated otherwise, an alkoxy is optionally substituted by one or more of OH, halo, CN, NO₂, C₁₋₆alkyl, C₃₋₈cycloalkyl, heterocycloalkyl, aryl, or heteroaryl.

“Aryl” refers to an unsaturated ring, having six to ten ring atoms (C₆₋₁₀aryl). In some embodiments, an aryl contains 6-10 ring atoms. In other embodiments, an aryl contains 6-8 ring atoms. Aryl also includes monocyclic or fused-ring polycyclic. Examples of aryl include, without limitation, phenyl, naphthyl, or indolyl. Unless stated otherwise, an aryl is optionally substituted by one or more of OH, halo, CN, NO₂, C₁₋₆alkyl, C₃₋₈cycloalkyl, heterocycloalkyl, aryl, or heteroaryl.

“Heteroaryl” refers to a 5-to 18-membered aromatic radical (e.g., (C₅₋₁₃)heteroaryl) that includes one or more ring heteroatoms (nitrogen, oxygen and/or sulfur) and is a monocyclic, bicyclic, tricyclic or tetracyclic ring system. In some embodiments, a heteroaryl contains 5-12 ring atoms. In further embodiments, a heteroaryl contains 5-10 ring atoms. In other embodiments, a heteroaryl contains 5-8 ring atoms. In still further embodiments, a heteroaryl contains 5-6 ring atoms. A polycyclic heteroaryl may be fused or non-fused. The heteroatom(s) in the heteroaryl are optionally oxidized and nitrogen atoms, if present, are optionally quaternized. The heteroaryl may be attached to the rest of the molecule through any atom of the ring(s). Examples of heteroaryls include, but are not limited to, benzimidazolyl, benzindolyl, benzofuranyl, benzothiazolyl, benzothiadiazolyl, benzo[b][1,4]dioxepinyl, benzo[1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl, benzothieno[3,2-d]pyrimidinyl, benzotriazolyl, carbazolyl, cinnolinyl, cyclopenta[d]pyrimidinyl, dibenzofuranyl, dibenzothiophenyl, furanyl, furazanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, 1,6-naphthyridinonyl, oxadiazolyl, oxoazepinyl, oxazolyl, oxiranyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyridopyrimidinyl, pyrazinyl, pyrimidinyl, pyridazinyl, pyrrolyl, quinazolinyl, quinoxalinyl, quinolinyl, isoquinolinyl, tetrahydroquinolinyl, thiazolyl, thiadiazolyl, thiapyranyl, triazolyl, tetrazolyl, triazinyl, or thiophenyl (i.e. thienyl). Unless stated otherwise, heteroaryl is optionally substituted by one or more of OH, halo, CN, NO₂, C₁₋₆alkyl, C₃₋₈cycloalkyl, heterocycloalkyl, aryl, or heteroaryl.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Subject” as used herein refers to a mammalian animal. In one embodiment, the patient or subject is a human. In another embodiment, the patient or subject is a veterinary or farm animal, a domestic animal or pet, or animal normally used for clinical research. In further embodiments, the subject is a canine, feline, or primate. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

Compounds described herein may include one or more chiral centers. Compounds described herein, therefore, may refer to a specific enantiomer or diastereomer. Compounds described herein may also be provided as mixtures of enantiomers or diastereomers.

Compounds of the disclosure may include all isotopes of any atom present in the compound. For example, one or more hydrogen atoms can be substituted with deuterium or tritium. Synthetic methods for preparing isotopes are generally known in the art.

### Compounds

Provided herein are compounds of Formula (I):

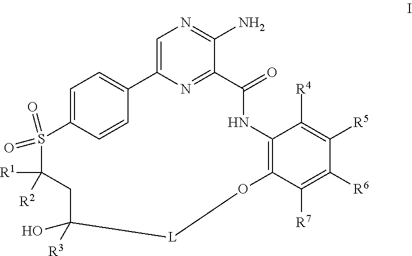

According to the disclosure, R¹ is H, C₁₋₆alkyl, or substituted C₁₋₆alkyl. In some embodiments, R¹ is H. In other embodiments, R¹ is C₁₋₆alkyl. For example, R¹ is methyl, ethyl, propyl, butyl, pentyl, or hexyl. In further embodiments, R¹ is methyl. In other embodiments, R¹ is ethyl. In yet further embodiments, R¹ is propyl. In still other embodiments, R¹ is butyl In further embodiments, R¹ is pentyl. In other embodiments, R¹ is hexyl. Optionally, R¹ is substituted, i.e., substituted C₁₋₆alkyl. In some embodiments, R¹ is substituted C₁₋₆alkyl. In other embodiments, R¹ is substituted methyl. In further embodiments, R¹ is substituted ethyl. In yet other embodiments, R¹ is substituted propyl. In still further embodiments, R¹ is substituted butyl. In other embodiments, R¹ is substituted pentyl. In further embodiments, R¹ is substituted hexyl.

According to the disclosure, R² is H, C₁₋₆alkyl, or substituted C₁₋₆alkyl. In some embodiments, R² is H. In other embodiments, R² is C₁₋₆alkyl. For example, R² is methyl, ethyl, propyl, butyl, pentyl, or hexyl. In further embodiments, R² is methyl. In other embodiments, R² is ethyl. In yet further embodiments, R² is propyl. In still other embodiments, R² is butyl In further embodiments, R² is pentyl. In other embodiments, R² is hexyl. Optionally, R² is substituted, i.e., substituted C₁₋₆alkyl. In some embodiments, R² is substituted C₁₋₆alkyl. In other embodiments, R² is substituted methyl. In further embodiments, R² is substituted ethyl. In yet other embodiments, R² is substituted propyl. In still further embodiments, R² is substituted butyl. In other embodiments, R² is substituted pentyl. In further embodiments, R² is substituted hexyl.

Alternatively, R¹ and R² are joined to form an optionally substituted C₃₋₈ cycloalkyl or an optionally substituted heterocycloalkyl. In some embodiments, R¹ and R² are joined to form an unsubstituted C₃₋₆cycloalkyl. For example, R¹ and R² are joined to form unsubstituted cyclopropyl. In certain aspects R¹ and R² are joined to form unsubstituted cyclobutyl. In other aspects, R¹ and R² are joined to form unsubstituted cyclopentyl. In further aspects, R¹ and R² are joined to form unsubstituted cyclohexyl. In other embodiments, R¹ and R² are joined to form a substituted C₃₋₆cycloalkyl. For example, R¹ and R² are joined to form substituted cyclopropyl, substituted cyclobutyl, substituted cyclopentyl, or substituted cyclohexyl. In certain aspects, R¹ and R² are joined to form substituted cyclopropyl. In other aspects, R¹ and R² are joined to form substituted cyclobutyl In further aspects, R¹ and R² are joined to form substituted cyclopentyl. In yet other aspects, R¹ and R² are joined to form substituted cyclohexyl. In further embodiments, R¹ and R² are joined to form an unsubstituted heterocycloalkyl. For example R¹ and R² are joined to form an unsubstituted pyrrolidinyl, unsubstituted tetrahydrofuranyl, unsubstituted oxetanyl, unsubstituted pyranyl, unsubstituted piperidinyl, or unsubstituted azetidinyl. In certain aspects, R¹ and R² are joined to form an unsubstituted pyrrolidinyl. In other aspects, R¹ and R² are joined to form an unsubstituted tetrahydrofuranyl. In further aspects, R¹ and R² are joined to form an unsubstituted oxetanyl. In yet other aspects, R¹ and R² are joined to form an unsubstituted pyranyl. In still further aspects, R¹ and R² are joined to form an unsubstituted piperidinyl. In other aspects, R¹ and R² are joined to form an unsubstituted azetidinyl. In yet other embodiments, R¹ and R² are joined to form substituted heterocycloalkyl. For example R¹ and R² are joined to form a substituted pyrrolidinyl, substituted tetrahydrofuranyl, substituted oxetanyl, substituted pyranyl, substituted piperidinyl, or substituted azetidinyl. In certain aspects, R¹ and R² are joined to form a substituted pyrrolidinyl. In other aspects, R¹ and R² are joined to form a substituted tetrahydrofuranyl. In further aspects, R¹ and R² are joined to form a substituted oxetanyl. In yet other aspects, R¹ and R² are joined to form a substituted pyranyl. In still further aspects, R¹ and R² are joined to form a substituted piperidinyl. In other aspects, R¹ and R² are joined to form a substituted azetidinyl.

According to the disclosure, R³ is H, C₁₋₆alkyl, or substituted C₁₋₆alkyl. In some embodiments, R³ is H. In other embodiments, R³ is C₁₋₆alkyl. For example, R³ is methyl, ethyl, propyl, butyl, pentyl, or hexyl. In further embodiments, R³ is methyl. In other embodiments, R³ is ethyl. In yet further embodiments, R³ is propyl. In still other embodiments, R³ is butyl In further embodiments, R³ is pentyl. In other embodiments, R³ is hexyl. Optionally, R³ is substituted, i.e., substituted C₁₋₆alkyl. In some embodiments, R³ is substituted C₁₋₆alkyl. In other embodiments, R³ is substituted methyl. In further embodiments, R³ is substituted ethyl. In yet other embodiments, R³ is substituted propyl. In still further embodiments, R³ is substituted butyl. In other embodiments, R³ is substituted pentyl. In further embodiments, R³ is substituted hexyl.

According to the disclosure, R⁴ is H, C₁₋₆alkyl, or substituted C₁₋₆alkyl. In some embodiments, R⁴ is H. In other embodiments, R⁴ is C₁₋₆alkyl. For example, R⁴ is methyl, ethyl, propyl, butyl, pentyl, or hexyl. In further embodiments, R⁴ is methyl. In other embodiments, R⁴ is ethyl. In yet further embodiments, R⁴ is propyl. In still other embodiments, R⁴ is butyl In further embodiments, R⁴ is pentyl. In other embodiments, R⁴ is hexyl. Optionally, R⁴ is substituted, i.e., substituted C₁₋₆alkyl. In some embodiments, R⁴ is substituted C₁₋₆alkyl. In other embodiments, R⁴ is substituted methyl. In further embodiments, R⁴ is substituted ethyl. In yet other embodiments, R⁴ is substituted propyl. In still further embodiments, R⁴ is substituted butyl. In other embodiments, R⁴ is substituted pentyl. In further embodiments, R⁴ is substituted hexyl. In certain aspects, R⁴ is substituted with NH₂, NH(C₁₋₆alkyl), or NH(C₁₋₆alkyl)(C₁₋₆alkyl). In other aspects, R⁴ is substituted with NH₂. In further aspects, R⁴ is substituted with NH(C₁₋₆alkyl). In yet other aspects, R⁴ is substituted with NH(C₁₋₆alkyl)(C₁₋₆alkyl). For example, R⁴ is substituted with NHCH₃, NHCH₂CH₃, N(CH₃)₂, or N(CH₂CH₃)₂. In certain aspects, R⁴ is substituted with NHCH₃. In further aspects, R⁴ is substituted with NHCH₂CH₃. In yet other aspects, R⁴ is substituted with N(CH₃)₂. In still further aspects, R⁴ is substituted with N(CH₂CH₃)₂.

According to the disclosure, R⁵ is H, C₁₋₆alkyl, or substituted C₁₋₆alkyl. In some embodiments, R⁵ is H. In other embodiments, R⁵ is C₁₋₆alkyl. For example, R⁵ is methyl, ethyl, propyl, butyl, pentyl, or hexyl. In further embodiments, R⁵ is methyl. In other embodiments, R⁵ is ethyl. In yet further embodiments, R⁵ is propyl. In still other embodiments, R⁵ is butyl In further embodiments, R⁵ is pentyl. In other embodiments, R³ is hexyl. Optionally, R⁵ is substituted, i.e., substituted C₁₋₆alkyl. In some embodiments, R⁵ is substituted C₁₋₆alkyl. In other embodiments, R⁵ is substituted methyl. In further embodiments, R⁵ is substituted ethyl. In yet other embodiments, R⁵ is substituted propyl. In still further embodiments, R⁵ is substituted butyl. In other embodiments, R³ is substituted pentyl. In further embodiments, R⁵ is substituted hexyl. In certain aspects, R⁵ is substituted with NH₂, NH(C₁₋₆alkyl), or NH(C₁₋₆alkyl)(C₁₋₆alkyl). In other aspects, R⁵ is substituted with NH₂. In further aspects, R⁵ is substituted with NH(C₁₋₆alkyl). In yet other aspects, R⁵ is substituted with NH(C₁₋₆alkyl)(C₁₋₆alkyl). For example, R is substituted with NHCH₃, NHCH₂CH₃, N(CH₃)₂, or N(CH₂CH₃)₂. In certain aspects, R⁵ is substituted with NHCH₃. In further aspects, R³ is substituted with NHCH₂CH₃. In yet other aspects, R⁵ is substituted with N(CH₃)₂. In still further aspects, R⁵ is substituted with N(CH₂CH₃)₂.

According to the disclosure, R⁶ is H, C₁₋₆alkyl, or substituted C₁₋₆alkyl. In some embodiments, R⁶ is H. In other embodiments, R⁶ is C₁₋₆alkyl. For example, R⁶ is methyl, ethyl, propyl, butyl, pentyl, or hexyl. In further embodiments, R⁶ is methyl. In other embodiments, R⁶ is ethyl. In yet further embodiments, R⁶ is propyl. In still other embodiments, R⁶ is butyl In further embodiments, R⁶ is pentyl. In other embodiments, R⁶ is hexyl. Optionally, R⁶ is substituted, i.e., substituted C₁₋₆alkyl. In some embodiments, R⁶ is substituted C₁₋₆alkyl. In other embodiments, R⁶ is substituted methyl. In further embodiments, R⁶ is substituted ethyl. In yet other embodiments, R⁶ is substituted propyl. In still further embodiments, R⁶ is substituted butyl. In other embodiments, R⁶ is substituted pentyl. In further embodiments, R⁶ is substituted hexyl. In certain aspects, R⁶ is substituted with NH₂, NH(C₁₋₆alkyl), or NH(C₁₋₆alkyl)(C₁₋₆alkyl). In other aspects, R⁶ is substituted with NH₂. In further aspects, R⁶ is substituted with NH(C₁₋₆alkyl). In yet other aspects, R⁶ is substituted with NH(C₁₋₆alkyl)(C₁₋₆alkyl). For example, R⁶ is substituted with NHCH₃, NHCH₂CH₃, N(CH₃)₂, or N(CH₂CH₃)₂. In certain aspects, R⁶ is substituted with NHCH₃. In further aspects, R⁶ is substituted with NHCH₂CH₃. In yet other aspects, R⁶ is substituted with N(CH₃)₂. In still further aspects, R⁶ is substituted with N(CH₂CH₃)₂.

According to the disclosure, R⁷ is H, C₁₋₆alkyl, or substituted C₁₋₆alkyl. In some embodiments, R⁷ is H. In other embodiments, R⁷ is C₁₋₆alkyl. For example, R⁷ is methyl, ethyl, propyl, butyl, pentyl, or hexyl. In further embodiments, R⁷ is methyl. In other embodiments, R⁷ is ethyl. In yet further embodiments, R⁷ is propyl. In still other embodiments, R⁷ is butyl In further embodiments, R⁷ is pentyl. In other embodiments, R⁷ is hexyl. Optionally, R⁷ is substituted, i.e., substituted C₁₋₆alkyl. In some embodiments, R⁷ is substituted C₁₋₆alkyl. In other embodiments, R⁷ is substituted methyl. In further embodiments, R⁷ is substituted ethyl. In yet other embodiments, R⁷ is substituted propyl. In still further embodiments, R⁷ is substituted butyl. In other embodiments, R⁷ is substituted pentyl. In further embodiments, R⁷ is substituted hexyl. In certain aspects, R⁷ is substituted with NH₂, NH(C₁₋₆alkyl), or NH(C₁₋₆alkyl)(C₁₋₆alkyl). In other aspects, R⁷ is substituted with NH₂. In further aspects, R⁷ is substituted with NH(C₁₋₆alkyl). In yet other aspects, R⁷ is substituted with NH(C₁₋₆alkyl)(C₁₋₆alkyl). For example, R⁷ is substituted with NHCH₃, NHCH₂CH₃, N(CH₃)₂, or N(CH₂CH₃)₂. In certain aspects, R⁷ is substituted with NHCH₃. In further aspects, R⁷ is substituted with NHCH₂CH₃. In yet other aspects, R⁷ is substituted with N(CH₃)₂. In still further aspects, R⁷ is substituted with N(CH₂CH₃)₂.

According to the disclosure, L is C₁₋₂₀alkylene, wherein one or more carbon atoms of the C₁₋₂₀alkylene are each optionally replaced with an oxygen atom. In some embodiments, L is unsubstituted C₁₋₂₀alkylene. In other embodiments, L is substituted C₁₋₂₀alkylene. In further embodiments, L is C₁₋₁₈alkylene, C₁₋₁₆alkylene, C₁₋₁₄alkylene. C₁₋₁₂alkylene, C₁₋₁₀alkylene, C₁₋₈alkylene, C₁₋₆alkylene, or C₁₋₄alkylene. In certain aspects, L is C₁₋₁₈alkylene. In other aspects, L is C₁₋₁₆alkylene. In further aspects, L is C₁₋₁₄alkylene. In yet other aspects, L is C₁₋₁₂alkylene. In still further aspects, L is C₁₋₁₀alkylene. In other aspects, L is C₁₋₈alkylene. In further aspects, L is C₁₋₄alkylene. In yet other aspects, L is C₁₋₄alkylene. In other embodiments, L comprises one or more oxygen atoms. In yet further embodiments, L comprises two oxygen atoms, three oxygen atoms, four oxygen atoms, five oxygen atoms, or six oxygen atoms. In certain aspects, L comprises one oxygen atom. In other aspects, L comprises two oxygen atoms. In further aspects, L comprises three oxygen atoms. In yet other aspects, L comprises four oxygen atoms. In still further aspects, L comprises five oxygen atoms. In other aspects, L comprises six oxygen atoms. In some embodiments, L is —(CH₂)₆—. In further embodiments, L is —CH₂O(CH₂)₄—. In other embodiments, L is —CH₂O(CH₂)₂O(CH₂)₂—

The disclosure also provides pharmaceutically acceptable salts of the compounds of Formula (I). “Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like. In some embodiments, the compounds are hydrochloride (HCl) salts.

In some embodiments, the compound is:

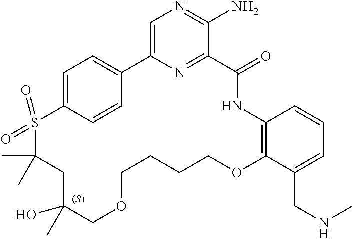

or a pharmaceutically acceptable salt thereof;

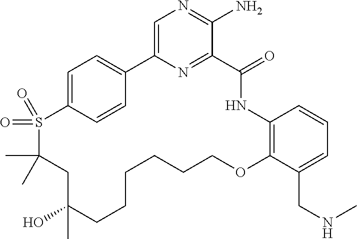

or a pharmaceutically acceptable salt thereof;

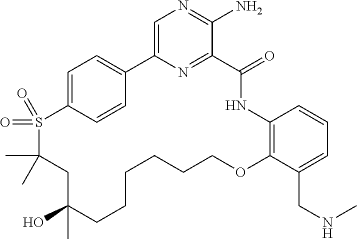

or a pharmaceutically acceptable salt thereof;

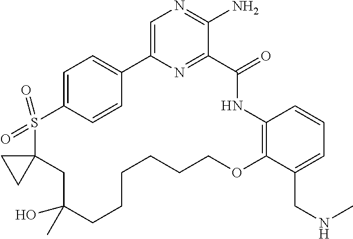 [SMILES: CNCc1cccc2c1OCCCCCCC(C)(O)CC1(CC1)S(=O)(=O)c1ccc(cc1)-c1cnc(N)c(n1)C(=O)N2]

or a pharmaceutically acceptable salt thereof;

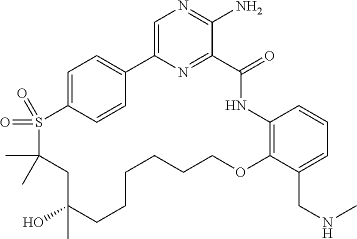

or a pharmaceutically acceptable salt thereof;

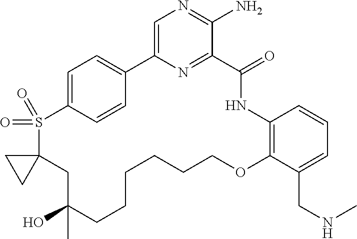 [SMILES: CNCc1cccc2c1OCCCCCC[C@@](C)(O)CC1(CC1)S(=O)(=O)c1ccc(cc1)-c1cnc(N)c(n1)C(=O)N2]

or a pharmaceutically acceptable salt thereof;

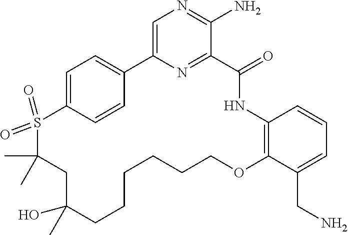 [SMILES: CC1(O)CCCCCCOc2c(CN)cccc2NC(=O)c2nc(cnc2N)-c2ccc(cc2)S(=O)(=O)C(C)(C)C1]

or a pharmaceutically acceptable salt thereof;

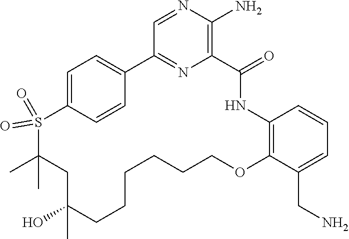 [SMILES: CC1(C)C[C@@](C)(O)CCCCCCOc2c(CN)cccc2NC(=O)c2nc(cnc2N)-c2ccc(cc2)S1(=O)=O]

or a pharmaceutically acceptable salt thereof;

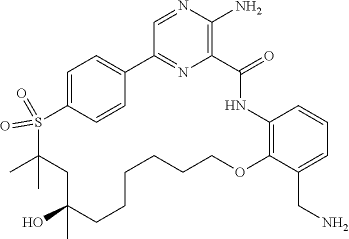 [SMILES: CC1(C)C[C@](C)(O)CCCCCCOc2c(CN)cccc2NC(=O)c2nc(cnc2N)-c2ccc(cc2)S1(=O)=O]

or a pharmaceutically acceptable salt thereof;

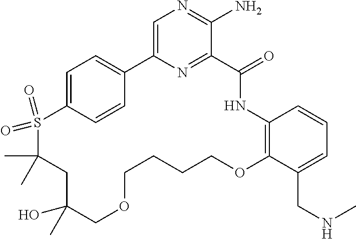

or a pharmaceutically acceptable salt thereof;

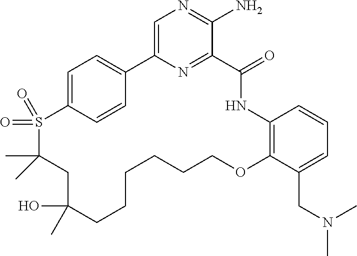

or a pharmaceutically acceptable salt thereof;

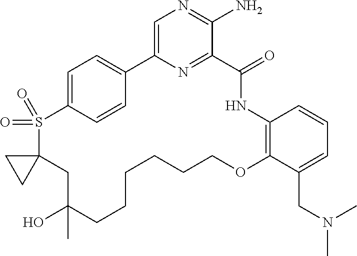 [SMILES: CN(C)Cc1cccc2c1OCCCCCCC(C)(O)CC1(CC1)S(=O)(=O)c1ccc(cc1)-c1cnc(N)c(n1)C(=O)N2]

or a pharmaceutically acceptable salt thereof;

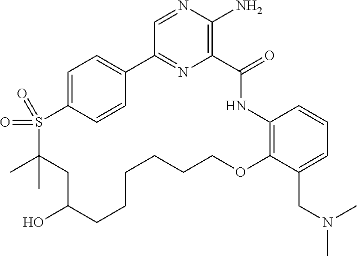 [SMILES: CN(C)Cc1cccc2c1OCCCCCCC(O)CC(C)(C)S(=O)(=O)c1ccc(cc1)-c1cnc(N)c(n1)C(=O)N2]

or a pharmaceutically acceptable salt thereof;

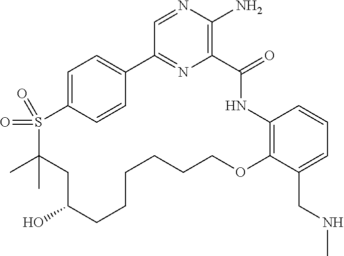 [SMILES: CNCc1cccc2c1OCCCCCC[C@H](O)CC(C)(C)S(=O)(=O)c1ccc(cc1)-c1cnc(N)c(n1)C(=O)N2]

or a pharmaceutically acceptable salt thereof;

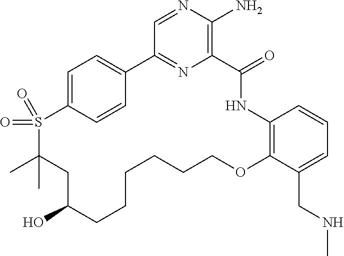 [SMILES: CNCc1cccc2c1OCCCCCC[C@@H](O)CC(C)(C)S(=O)(=O)c1ccc(cc1)-c1cnc(N)c(n1)C(=O)N2]

or a pharmaceutically acceptable salt thereof;

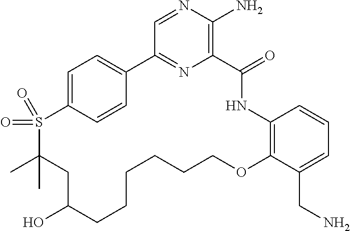 [SMILES: CC1(C)CC(O)CCCCCCOc2c(CN)cccc2NC(=O)c2nc(cnc2N)-c2ccc(cc2)S1(=O)=O]

or a pharmaceutically acceptable salt thereof;

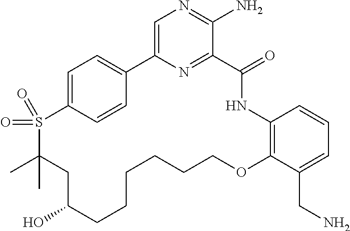 [SMILES: CC1(C)C[C@@H](O)CCCCCCOc2c(CN)cccc2NC(=O)c2nc(cnc2N)-c2ccc(cc2)S1(=O)=O]

or a pharmaceutically acceptable salt thereof;

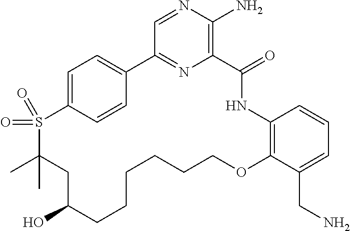 [SMILES: CC1(C)C[C@H](O)CCCCCCOc2c(CN)cccc2NC(=O)c2nc(cnc2N)-c2ccc(cc2)S1(=O)=O]

or a pharmaceutically acceptable salt thereof;

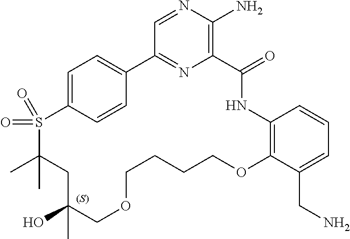

or a pharmaceutically acceptable salt thereof; or

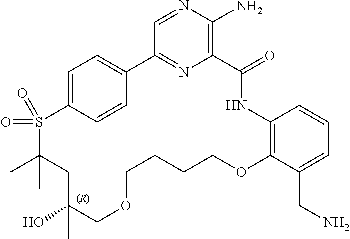

or a pharmaceutically acceptable salt thereof.

### Compositions

The compounds of the disclosure are used, alone or in combination with one or more additional active ingredients, to formulate pharmaceutical compositions of the disclosure. In some embodiments, the pharmaceutical composition comprises: (a) an effective amount of at least one compound in accordance with the disclosure; and (b) a pharmaceutically acceptable excipient.

The compositions or compounds may be administered by a suitable route of delivery, e.g., oral, parenteral, rectal, topical, or ocular routes, or by inhalation. In some embodiments, the compound or composition containing the compound is administered orally.

For oral administration, the compounds of the disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets or capsules may include a compound according to the disclosure mixed with pharmaceutically acceptable excipients such as inert diluents, disintegrating agents, binding agents, lubricating agents, sweetening agents, flavoring agents, coloring agents and preservative agents.

The compounds of this disclosure may also be administered by non-oral routes. For example, the compositions may be formulated for rectal administration as a suppository. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride.

### Methods of Treatment

The macrocyclic compounds, compositions containing the same and methods of treatment of the present disclosure have utility in treating many disease conditions, including cancer. In some embodiments, the compounds are useful for treating central nerve system cancer, breast cancer, pancreatic cancer, lung cancer, ovarian cancer, leukemia, lymphoma, melanoma, renal cancer, prostate cancer, colorectal cancer, brain cancer, and/or glioblastoma. In other embodiments, the methods treat ocular melanoma, desmoplastic round cell tumor, chondrosarcoma, leptomengial disease, diffuse large B-cell lymphoma, acute lymphoblastic leukemia, acute myeloid leukemia, adrenocortical carcinoma, AIDS-related cancer, AIDS-related lymphoma, anal cancer, rectal cancer, appendix cancer, astrocytomas, or atypical teratoid/rhabdoid tumor. In further embodiments, the methods treat basal cell carcinoma, bile duct cancer, bladder cancer, bone cancer, osteosarcoma, malignant fibrous histiocytoma, brain tumor, breast cancer, prostate cancer, bronchial tumor, Burkitt Lymphoma, or spinal cord tumor. In yet other embodiments, the methods treat carcinoid tumors, carcinoma of unknown primary, central nervous system atypical teratoid/rhabdoid tumor, leptomeningeal disease, central nervous system embryonal tumors, central nervous system lymphoma, cervical cancer, chordoma, chronic lymphocytic leukemia, chronic myelogenous leukemia, chronic myeloproliferative disorders, colon cancer, colorectal cancer, craniopharyngioma, or cutaneous T-cell lymphoma. In further embodiments, the methods treat endometrial cancer, ependymoblastoma, ependymoma, esophageal cancer, Ewing sarcoma family of tumors, extracranial germ cell tumor, extragonadal germ cell tumor, extrahepatic bile duct cancer, or eye cancer. In yet other embodiments, the methods treat gallbladder cancer, gastric (stomach) cancer, gastrointestinal carcinoid tumor, gastrointestinal stromal tumor (GIST), germ cell tumor, gestational trophoblastic tumor, or glioma. In yet other embodiments, the methods treat hairy cell leukemia, head and neck cancer, hepatocellular (liver) cancer, histiocytosis, Hodgkin lymphoma, or hypopharyngeal cancer. In still further embodiments, the methods treat Kaposi sarcoma or kidney (renal cell) cancer. In other embodiments, the methods treat Langerhans cell histiocytosis, laryngeal cancer, lip cancer, oral cavity cancer, liver cancer, lung cancer, Non-Hodgkin lymphoma, or primary central nervous system lymphoma. In further embodiments, the methods treat Waldenström's macroglobulinemia (lymphoplasmacytic lymphoma), malignant fibrous histiocytoma of bone, osteosarcoma, medulloblastoma, medulloepithelioma, melanoma, Merkel cell carcinoma, mesothelioma, metastatic squamous neck cancer with occult primary, multiple endocrine neoplasia syndrome, mouth cancer, multiple myeloma/plasma cell neoplasm, mycosis fungoides, myelodysplastic syndrome, myelodysplastic/myeloproliferative neoplasms, multiple myeloma, or myeloproliferative disorders. In other embodiments, the methods treat cancer. In further embodiments, the methods treat nasal cavity and paranasal sinus cancer, nasopharyngeal cancer, or neuroblastoma. In yet other embodiments, the methods treat oropharyngeal cancer, osteosarcoma and malignant fibrous histiocytoma of bone, ovarian cancer, ovarian germ cell tumor, ovarian epithelial cancer, or ovarian low malignant potential tumor. In still further embodiments, the methods treat pancreatic cancer, papillomatosis, paranasal sinus and nasal cavity cancer, parathyroid cancer, penile cancer, pharyngeal cancer, pineal parenchymal tumors of intermediate differentiation, pineoblastoma and supratentorial primitive neuroectodermal tumors, pituitary tumor, pleuropulmonary blastoma, pregnancy cancer, breast cancer, or prostate cancer. In other embodiments, the methods treat rectal cancer, renal cancer, pelvis cancer, ureter cancer, respiratory tract carcinoma involving the NUT gene on chromosome 15, retinoblastoma, or rhabdomyosarcoma. In further embodiments, the methods treat high grade prostate cancer. In yet other embodiments, the methods treat medium grade prostate cancer. In still further embodiments, the methods treat low grade prostate cancer. In other embodiments, the methods treat castration-resistant prostate cancer. In some embodiments, the cancer is breast cancer, prostate cancer, pancreatic cancer, lung cancer, colorectal cancer, ovarian cancer, liver cancer, melanoma, renal cancer, a central nervous system cancer, brain cancer, glioblastoma, a leukemia, or a lymphoma. In further embodiments, the methods treat salivary gland cancer, sarcoma, Sezary syndrome, skin cancer, ocular cancer, skin carcinoma, small intestine cancer, soft tissue sarcoma, squamous cell carcinoma, squamous neck cancer with occult primary, or supratentorial primitive neuroectodermal tumors. In yet other embodiments, the methods treat T-cell lymphoma, testicular cancer, throat cancer, thymoma and thymic carcinoma, thyroid cancer, transitional cell cancer of the renal, pelvis and ureter, or gestational trophoblastic tumor. In still further embodiments, the methods treat carcinoma of unknown primary site such as carcinoma of unknown primary site, unusual cancer of childhood, urethral cancer, or uterine sarcoma. In other embodiments, the methods treat vaginal cancer, vulvar cancer, Wilm's tumor, or a women's cancer. In further embodiments, the methods treat Wilm's tumor or a women's cancer. In yet other embodiments, the methods treat brain cancer, breast cancer, central nervous system cancer, colorectal cancer, glioblastoma, melanoma, leukemia, liver cancer, lung cancer, lymphoma, pancreatic cancer, prostate cancer, ovarian cancer, or renal cancer. In still further embodiments, the methods treat ocular melanoma, desmoplastic round cell tumor, chondrosarcoma, leptomengial disease, diffuse large B-cell lymphoma, acute lymphoblastic leukemia, acute myeloid leukemia, adrenocortical carcinoma, an AIDS-related cancer, an AIDS-related lymphoma, anal or rectal cancer, appendix cancer, astrocytomas, atypical teratoid/rhabdoid tumor, basal cell carcinoma, bile duct cancer, bladder cancer, bone cancer, osteosarcoma or malignant fibrous histiocytoma, brain tumor, breast cancer, prostate cancer, bronchial tumor, Burkitt lymphoma, spinal cord tumor, carcinoid tumor, carcinoma of unknown primary, central nervous system atypical teratoid/rhabdoid tumor, leptomeningeal disease, central nervous system embryonal tumors, central nervous system lymphoma, chordoma, chronic lymphocytic leukemia, chronic myelogenous leukemia, chronic myeloproliferative disorders, colon cancer, colorectal cancer, craniopharyngioma, cutaneous T-cell lymphoma, ependymoblastoma, ependymoma, esophageal cancer, a Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor (GIST), germ cell tumor, glioma, hairy cell leukemia, head or neck cancer, hepatocellular (liver) cancer, histiocytosis, Hodgkin's lymphoma, hypopharyngeal cancer, Kaposi sarcoma, kidney (renal) cancer, Langerhan's cell histiocytosis, laryngeal cancer, lip or oral cavity cancer, lung cancer, Non-Hodgkin's lymphoma, primary central nervous system lymphoma, Waldenström's macroglobulinemia (lymphoplasmacytic lymphoma), medulloblastoma, medulloepithelioma, melanoma, merkel cell carcinoma, mesothelioma, metastatic squamous neck cancer with occult primary, multiple endocrine neoplasia syndrome, mouth cancer, multiple myeloma/plasma cell neoplasm, mycosis fungoides, myelodysplastic syndromes, myelodysplastic/myeloproliferative neoplasms, multiple myeloma, myeloproliferative disorder, nasal cavity or paranasal sinus cancer, nasopharyngeal cancer, neuroblastoma, oral cancer, oropharyngeal cancer, osteosarcoma or malignant fibrous histiocytoma of bone, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, prostate cancer, rectal cancer, pelvis or ureter cancer, respiratory tract carcinoma involving the NUT gene on chromosome 15, retinoblastoma, rhabdomyosarcoma, high grade prostate cancer, medium grade prostate cancer, low grade prostate cancer, castration-resistant prostate cancer, salivary gland cancer, sarcoma, Sézary syndrome, skin cancer such as a skin carcinoma, ocular cancer, small intestine cancer, soft tissue sarcoma, squamous cell carcinoma, squamous neck cancer with occult primary, supratentorial primitive neuroectodermal tumors, T-cell lymphoma, testicular cancer, throat cancer, thymoma or thymic carcinoma, thyroid cancer, transitional cell cancer of the renal, pelvis or ureter, cancer of unknown primary site such as carcinoma of unknown primary site, unusual cancers of childhood, urethral cancer, Wilm's tumor, or a women's cancer such as breast cancer, cervical cancer, endometrial cancer, gestational trophoblastic tumor, ovarian cancer, ovarian germ cell tumor, ovarian epithelial cancer, ovarian low malignant potential tumor, pregnancy cancer, uterine sarcoma, vaginal cancer, or vulvar cancer.

The utility of the methods and compositions is not limited to any particular animal species. In at least one embodiment, a subject treated according to methods and using compositions, can be mammalian or non-mammalian. In at least one embodiment, a mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. In at least one embodiment, a non-mammalian subject can be any non-mammal including, but not limited to, a bird such as a duck, goose, chicken, or turkey. In at least one embodiment, subjects can be either gender and can be any age. In at least one embodiment, the compositions and methods can also be used to prevent cancer.

The compounds of the disclosure can also be used in combination with other therapeutic chemotherapy agents such as, e.g., enzyme inhibitors, PARP inhibitors, tyrosine kinase inhibitors, DNA binding agents, mitotic inhibitors, alkylating agents, anti-metabolites, anti-tumor antibiotics, topoisomerase inhibitors, microtubule inhibitors, angiogenesis inhibitors, signal transduction inhibitors, cell cycle inhibitors, bisphosphonates, telomerase inhibitors, biological response modifiers (such as antibodies, immunotherapy and peptide mimics), anti-hormones, anti-androgens, gene silencing agents, gene activating agents, and anti-vascular agents.

The compounds described herein may be prepared according to the procedures of, e.g., Schemes 1-7.

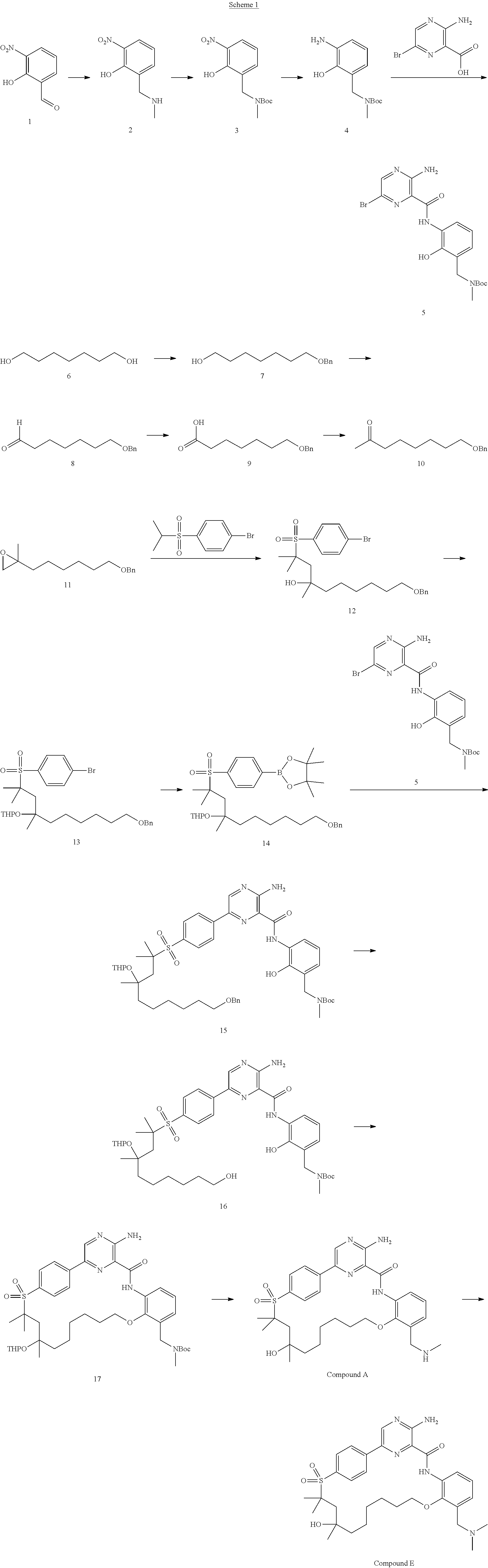

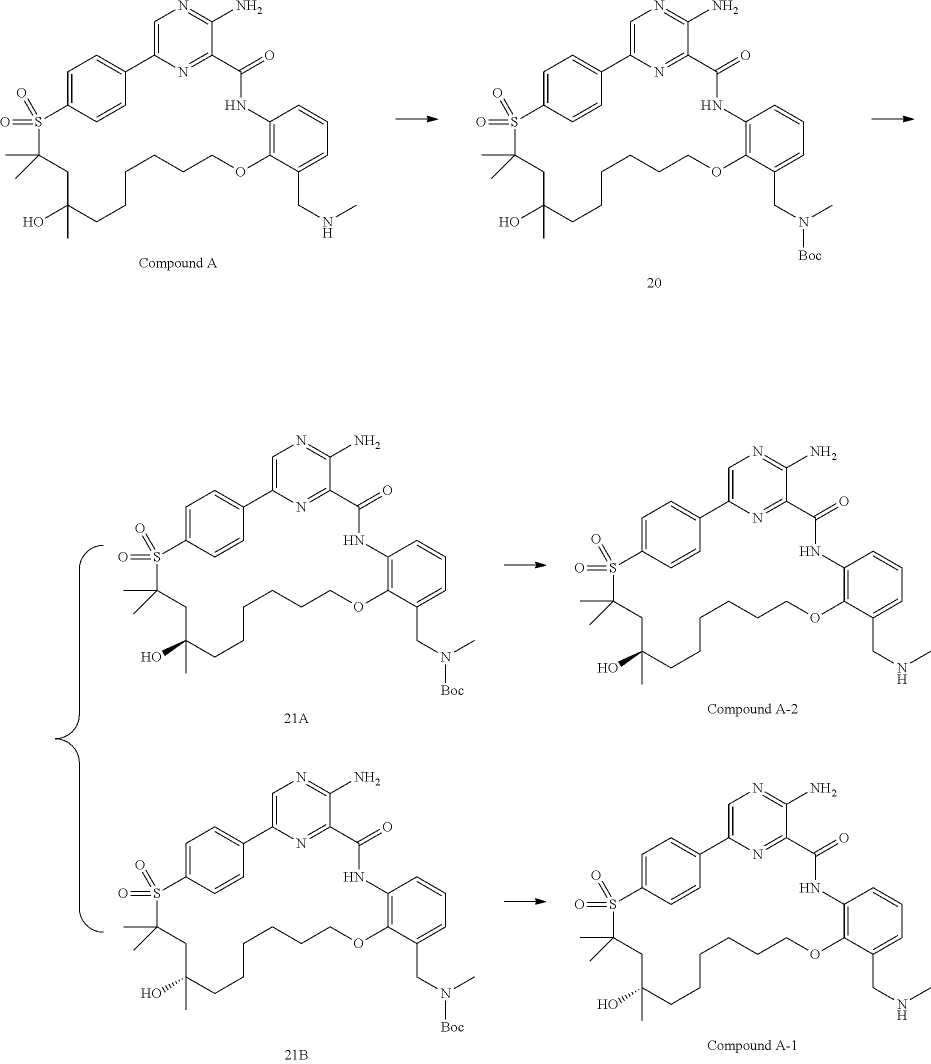

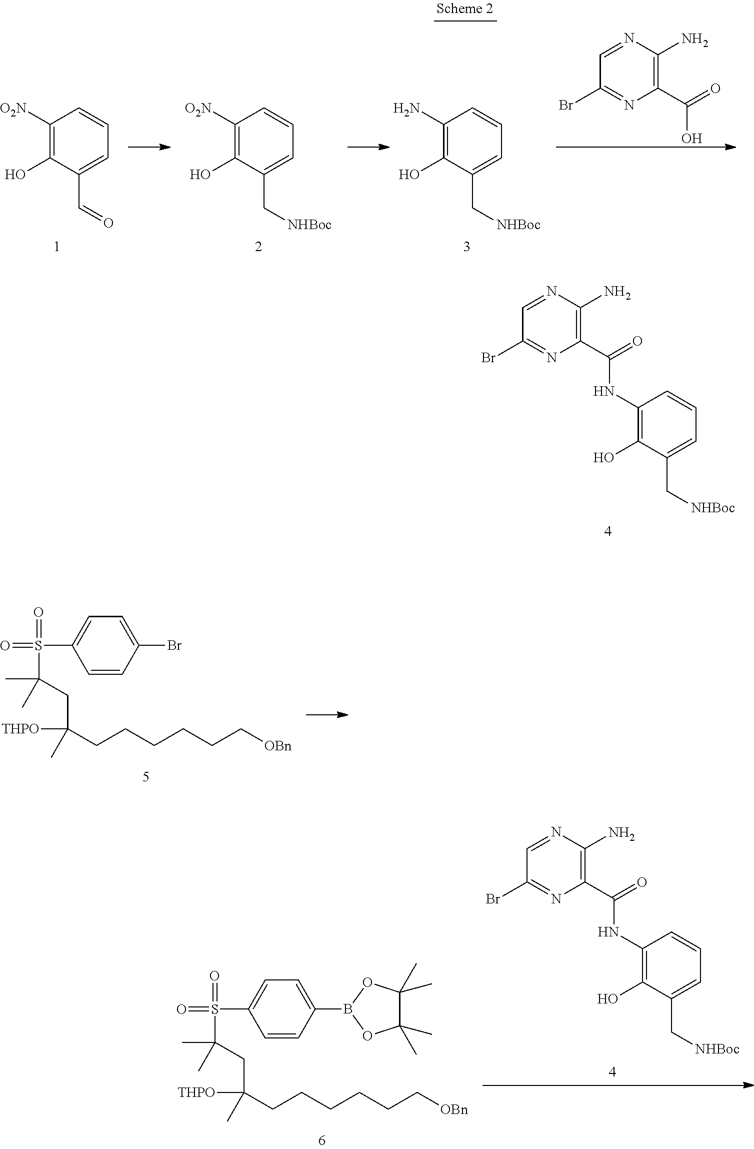

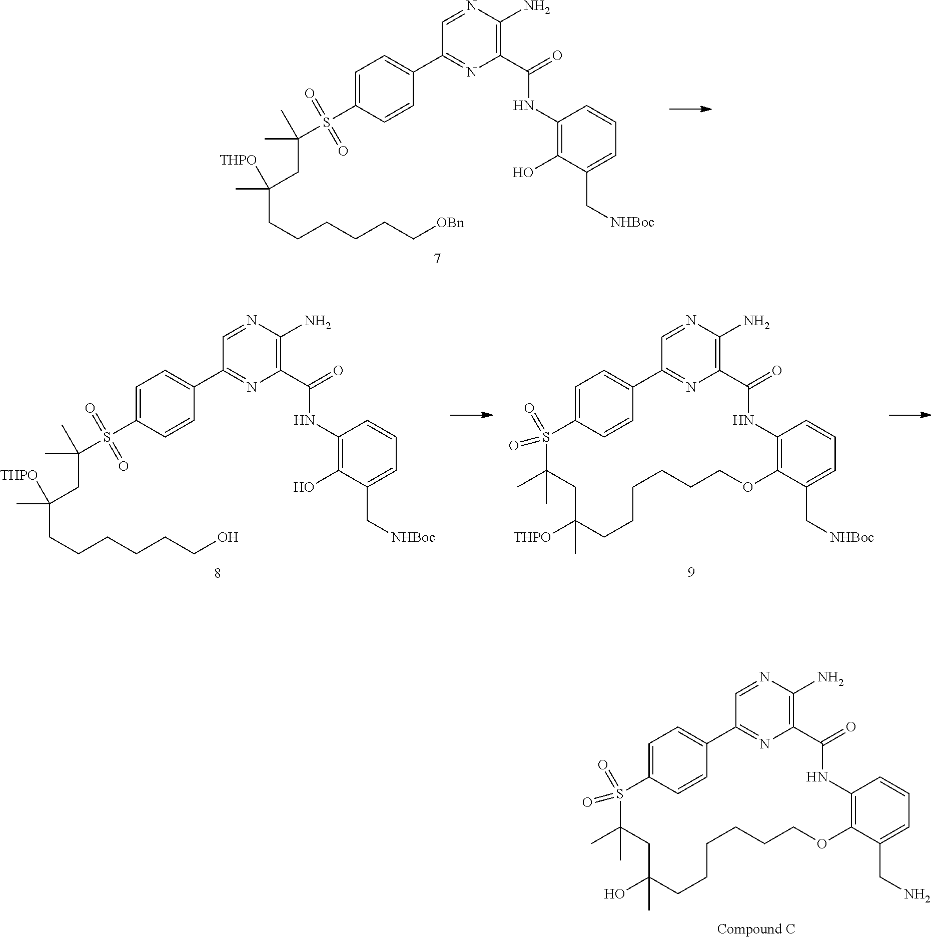

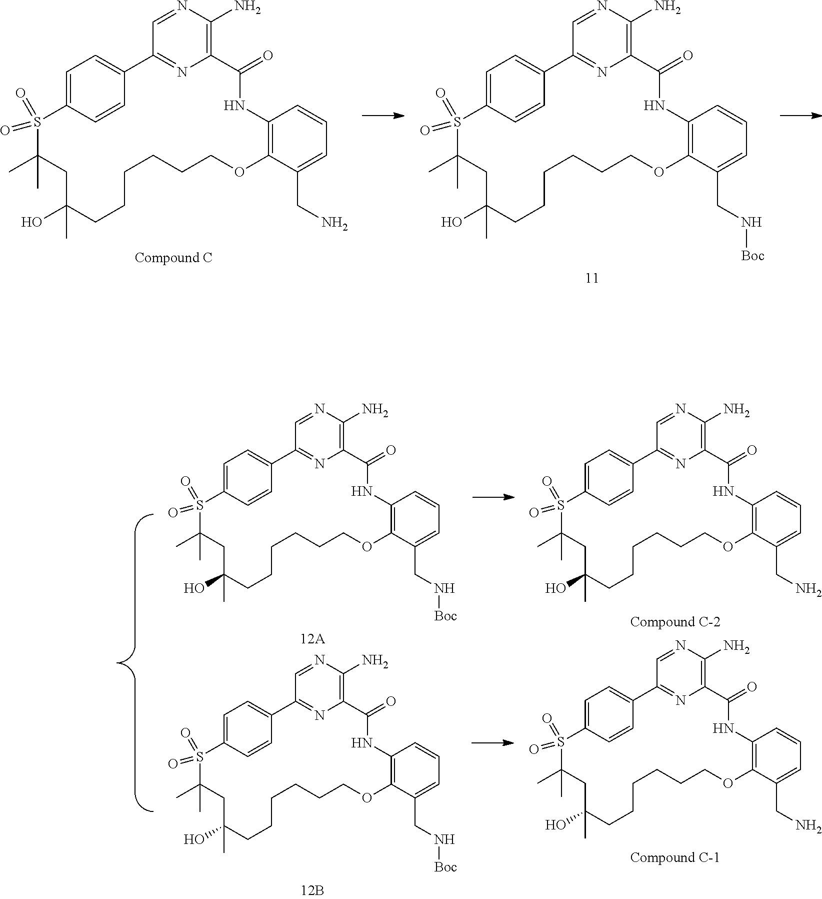 [SMILES: CC1(O)CCCCCCOc2c(CN)cccc2NC(=O)c2nc(cnc2N)-c2ccc(cc2)S(=O)(=O)C(C)(C)C1 ; CC1(O)CCCCCCOc2c(CNC(=O)OC(C)(C)C)cccc2NC(=O)c2nc(cnc2N)-c2ccc(cc2)S(=O)(=O)C(C)(C)C1 ; CC(C)(C)OC(=O)NCc1cccc2c1OCCCCCC[C@@](C)(O)CC(C)(C)S(=O)(=O)c1ccc(cc1)-c1cnc(N)c(n1)C(=O)N2 ; CC1(C)C[C@](C)(O)CCCCCCOc2c(CN)cccc2NC(=O)c2nc(cnc2N)-c2ccc(cc2)S1(=O)=O ; CC(C)(C)OC(=O)NCc1cccc2c1OCCCCCC[C@](C)(O)CC(C)(C)S(=O)(=O)c1ccc(cc1)-c1cnc(N)c(n1)C(=O)N2 ; CC1(C)C[C@@](C)(O)CCCCCCOc2c(CN)cccc2NC(=O)c2nc(cnc2N)-c2ccc(cc2)S1(=O)=O]

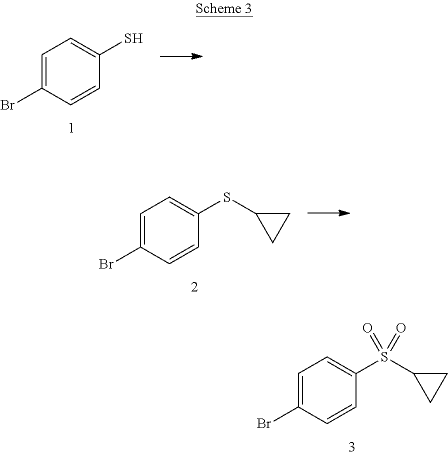 [SMILES: Sc1ccc(Br)cc1 ; Brc1ccc(SC2CC2)cc1 ; O=S(=O)(c1ccc(Br)cc1)C1CC1]

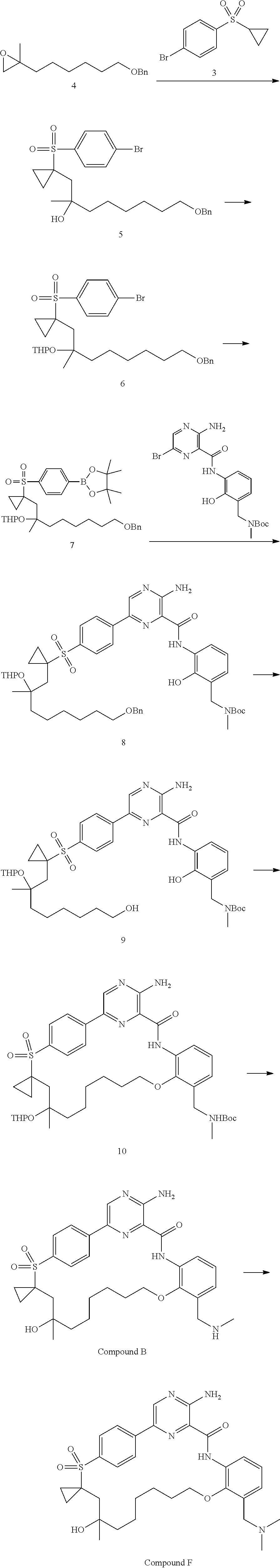

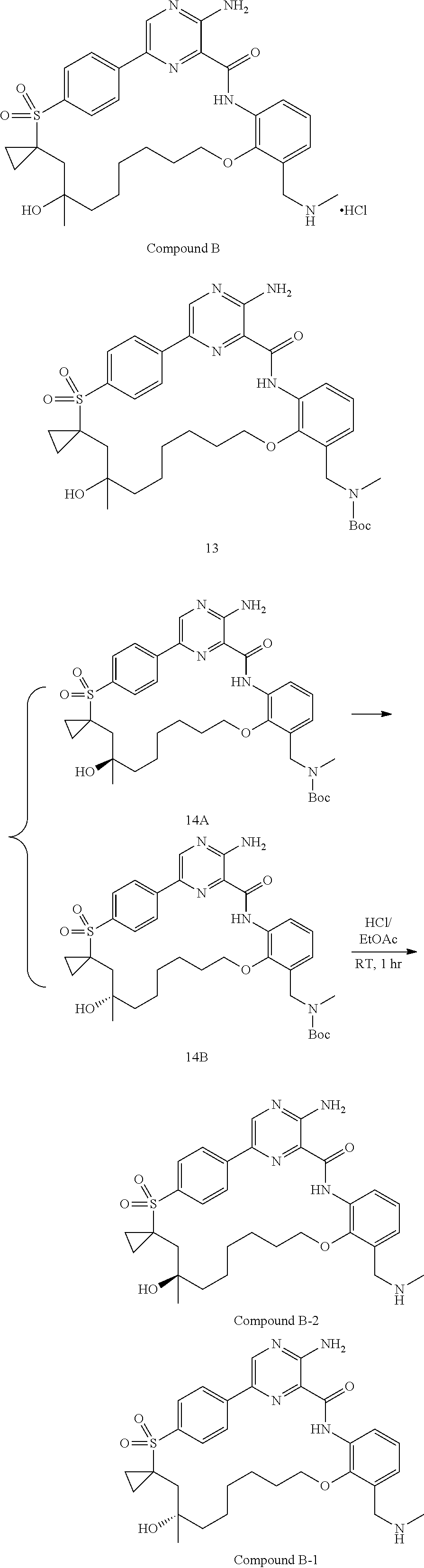

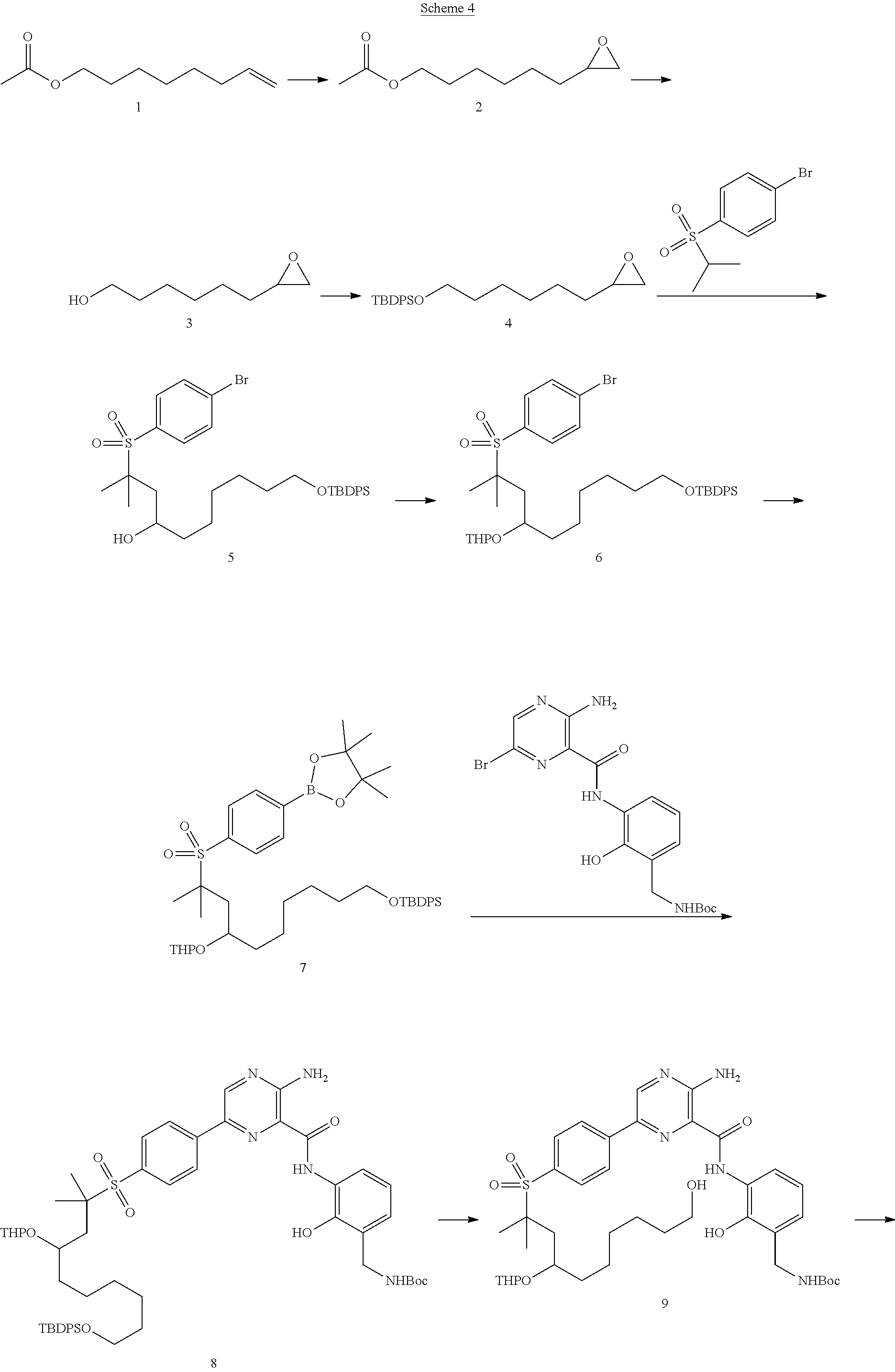

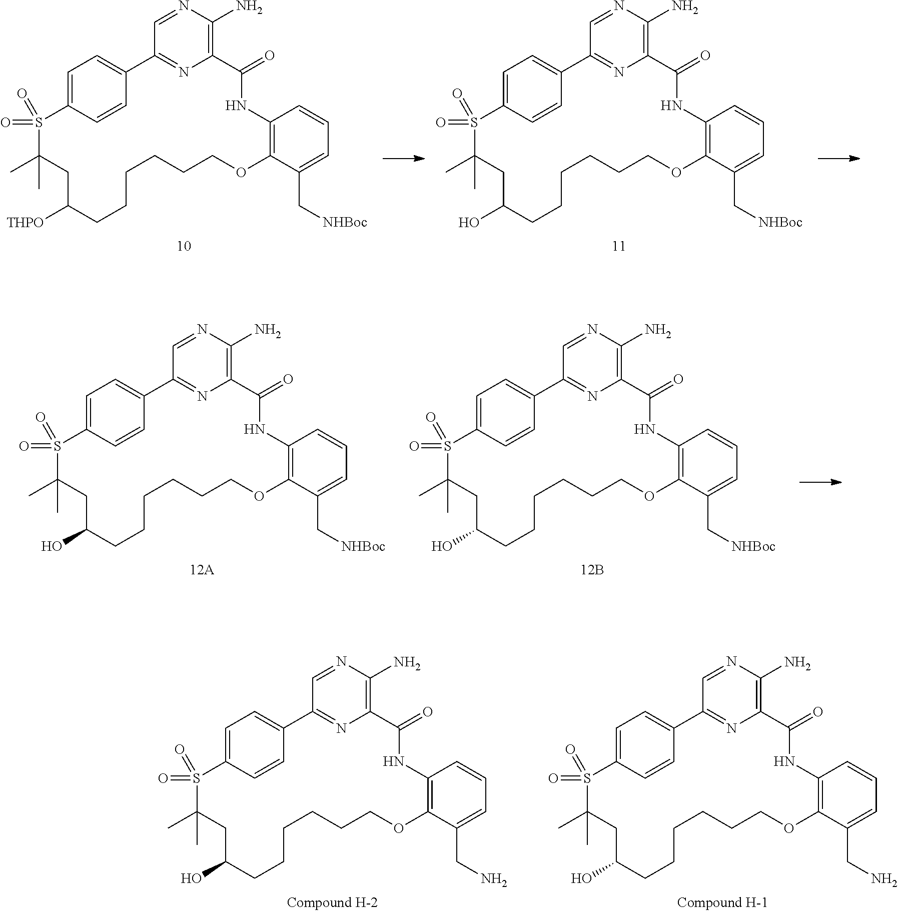

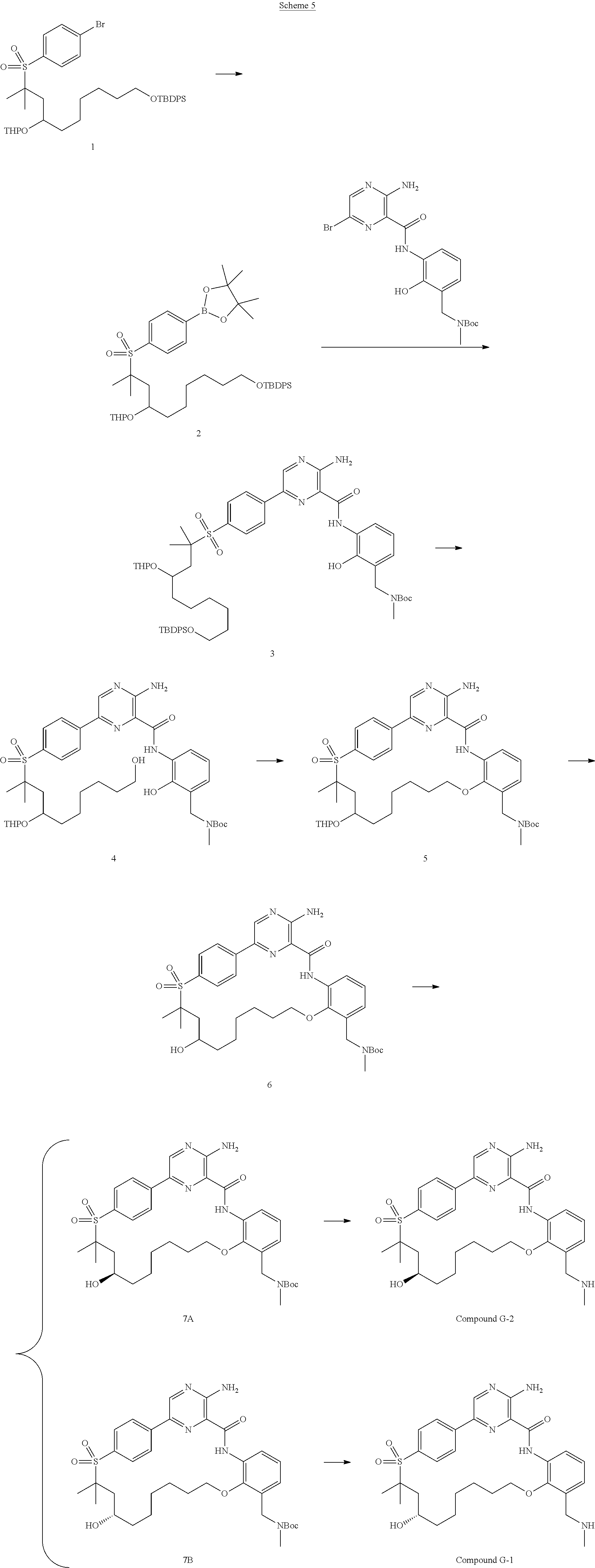

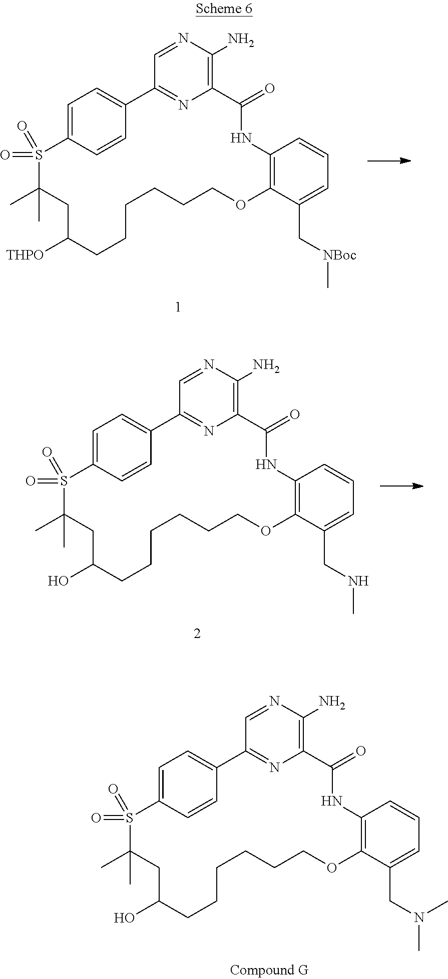

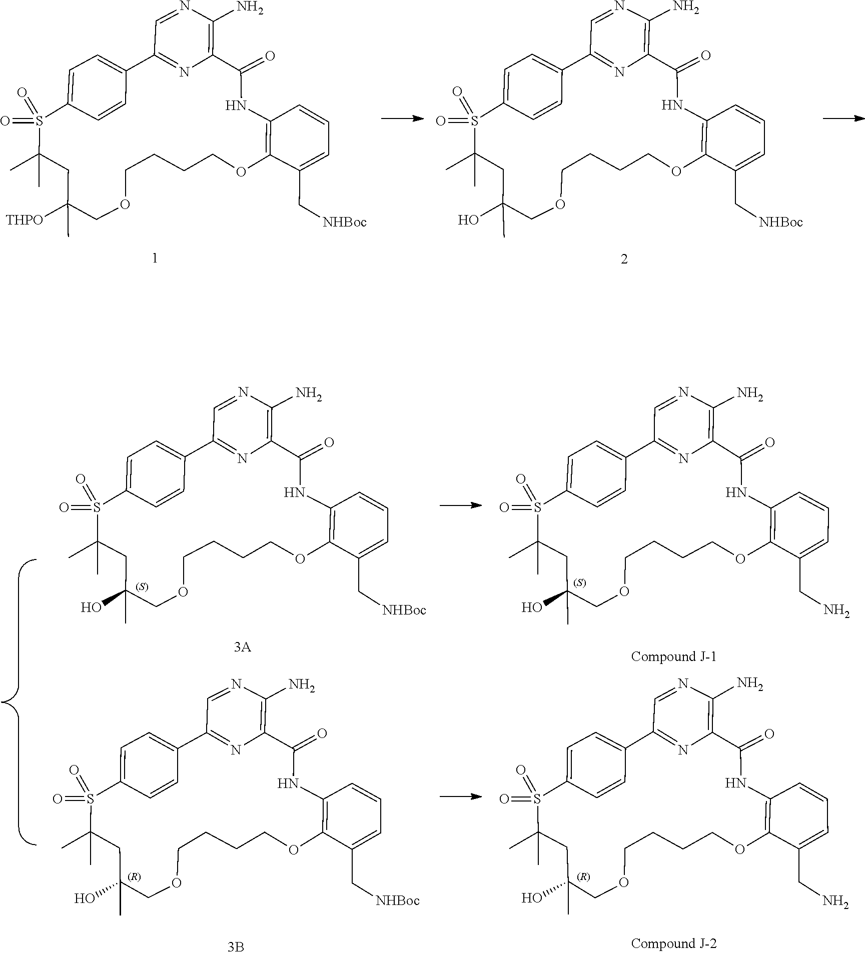

All references, including publications, patent applications, and patents, cited herein, are hereby incorporated by reference to the same extent as if each reference were individually and specifically indicated to be incorporated by reference and were set forth in its entirety herein.

### EXAMPLES

The following examples describe the preparation of representative compounds of the present invention. Melting points are reported as uncorrected in degrees centigrade. Mass spectral data is reported as the mass-to-charge ratio, m/z; and for high resolution mass spectral data, the calculated and experimentally found masses, [M⁺H]⁺, for the neutral formulae M are reported. Nuclear magnetic resonance data is reported as δ in parts per million (ppm) downfield from the standard, tetramethylsilane, along with the solvent, nucleus, and field strength parameters. The spin-spin homonuclear coupling constants are reported as J values in hertz; and the multiplicities are reported as: s, singlet; d, doublet; t, triplet; q, quartet; quintet; or br, broadened.

### Example 1: Preparation of Compound A

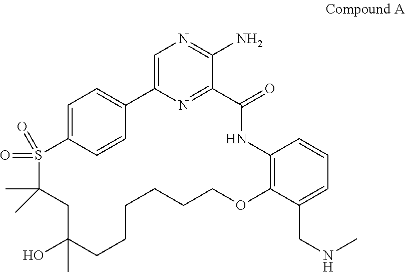 [SMILES: CNCc1cccc2c1OCCCCCCC(C)(O)CC(C)(C)S(=O)(=O)c1ccc(cc1)-c1cnc(N)c(n1)C(=O)N2]

### (i) Preparation of Compound 2

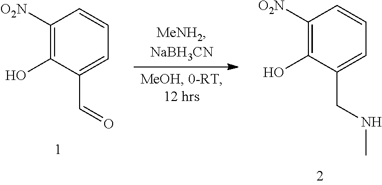

To a solution of 2-hydroxy-3-nitro-benzaldehyde (14 g, 83.77 mmol, 1.0 eq.) in MeOH (500 mL) was added MeNH₂ (45.04 g, 435.07 mmol, 30% purity, 5.19 eq.). The mixture was stirred at 20° C. for 20 mins. The reaction mixture was concentrated under reduced pressure to give a residue. Then the mixture was dissolved with MeOH (500 mL) and added NaBH₃CN (7.90 g, 125.66 mmol, 1.5 eq.) at 0° C. The mixture was stirred at 20° C. for 12 hours. The mixture was then quenched by sat. aq. Na₂CO₃ (100 mL) and extracted with EtOAc (30 mL×3). The combined organic layers were dried over Na₂SO₄, filtered and concentrated under reduced pressure to give 2-(methylaminomethyl)-6-nitro-phenol (15 g, crude) as an orange solid. ESI [M+H]=183.1.

### (ii) Preparation of Compound 3

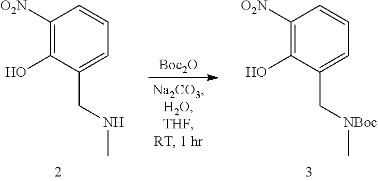 [SMILES: "Boc2O" ; CNCc1cccc([N+](=O)[O-])c1O ; CN(Cc1cccc([N+](=O)[O-])c1O)C(=O)OC(C)(C)C]

To a solution of 2-(methylaminomethyl)-6-nitro-phenol (15 g, 82.34 mmol, 1.0 eq.) in THF (300 mL) was added Na₂CO₃ (8.73 g, 82.34 mmol, 1.0 eq.) and H₂O (14.83 g, 823.38 mmol, 14.83 mL, 10.0 eq.) to pH=8, then Boc₂O (23.36 g, 107.04 mmol, 24.59 mL, 1.3 eq.) was added. The mixture was stirred at 20° C. for 1 hour. The reaction mixture was concentrated under reduced pressure to remove THF. The residue was diluted with sat. aq. KHSO₄ (100 mL) and extracted with ethyl acetate (300 mL×2), dried over Na₂SO₄, filtered and concentrated under reduced pressure to give a residue. The residue was purified by column chromatography (SiO₂, petroleum ether:ethyl acetate=100:1 to 5:1) to give tert-butyl N-[(2-hydroxy-3-nitro-phenyl)methyl]-N-methyl-carbamate (21 g, 74.39 mmol, 90.35% yield) as a yellow solid. ESI [M+H−tBu]=227.1.

(iii) Preparation of Compound 4

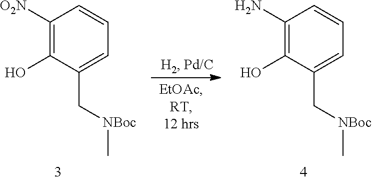 [SMILES: CN(Cc1cccc([N+](=O)[O-])c1O)C(=O)OC(C)(C)C ; "H2, Pd/C" ; CN(Cc1cccc(N)c1O)C(=O)OC(C)(C)C]

A mixture of tert-butyl N-[(2-hydroxy-3-nitro-phenyl)methyl]-N-methyl-carbamate (20.9 g, 74.04 mmol, 1.0 eq.), Pd/C (20.9 g, 10% purity) in EtOAc (600 mL) was degassed and purged with H₂ for 3 times, and then the mixture was stirred at 20° C. for 12 hours under H₂ (15 Psi) atmosphere. The reaction mixture was filtered, the filtrate was concentrated under reduced pressure to give tert-butyl N-[(3-amino-2-hydroxy-phenyl)methyl]-N-methyl-carbamate (18 g, crude) as an orange oil. ESI [M+H−tBu]=197.3.

### (iv) Preparation of Compound 5

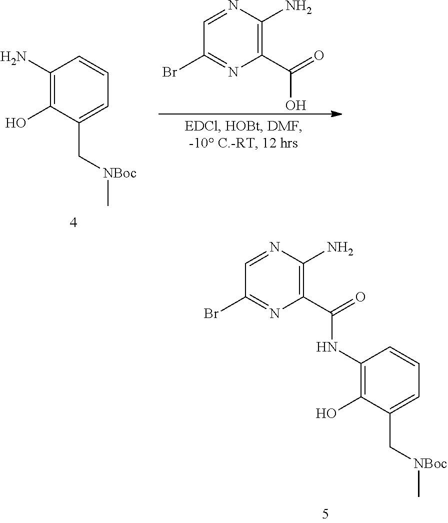 [SMILES: CN(Cc1cccc(N)c1O)C(=O)OC(C)(C)C ; Nc1ncc(Br)nc1C(=O)O ; CN(Cc1cccc(NC(=O)c2nc(Br)cnc2N)c1O)C(=O)OC(C)(C)C]

To a solution of tert-butyl N-[(3-amino-2-hydroxy-phenyl)methyl]-N-methyl-carbamate (18 g, 71.34 mmol, 1.05 eq.) and 3-amino-6-bromo-pyrazine-2-carboxylic acid (14.81 g, 67.94 mmol, 1.0 eq.) in DMF (300 mL) was added EDCI (15.63 g, 81.53 mmol, 1.2 eq.) and HOBt (4.59 g, 33.97 mmol, 0.5 eq.) at −10° C. The mixture was stirred at 20° C. for 12 hours. The reaction mixture was added H₂O (300 mL) and extracted with ethyl acetate (300 mL×2). The organic layer was washed with brine (200 mL×2), dried over Na₂SO₄, filtered and concentrated to give a residue. The residue was purified by column chromatography (SiO₂, petroleum ether:ethyl acetate=100:1 to 2:1) to give tert-butyl N-[[3-[(3-amino-6-bromo-pyrazine-2-carbonyl)amino]-2-hydroxy-phenyl]methyl]-N-methyl-carbamate (22 g, 47.16 mmol, 69.41% yield, 96.96% purity) as a yellow solid. ESI [M+H−Boc]=352.1/354.1.

### (v) Preparation of Compound 7

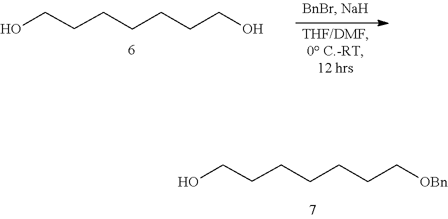 [SMILES: OCCCCCCCO ; OCCCCCCCOCc1ccccc1]

To a solution of heptane-1,7-diol (150 g, 1.13 mol, 1.0 eq.) in THF (3000 mL) and DMF (1000 mL) was added NaH (45.38 g, 1.13 mol, 60% purity, 1.0 eq.) at 0° C. The mixture was stirred at 20° C. for 1 hour, then BnBr (194.06 g, 1.13 mol, 134.77 mL, 1.0 eq.) was added at 0° C. The mixture was stirred at 20° C. for 11 hours. The reaction mixture was quenched with sat. aq. NH₄Cl (3 L) and extracted with Ethyl acetate (3 L×3). The organic layer was washed with brine (2 L), dried over Na₂SO₄, filtered and concentrated under reduced pressure to give a residue. The residue was purified by column chromatography (SiO₂, petroleum ether:ethyl acetate=100:1 to 2:1) to give 7-benzyloxyheptan-1-ol (401 g, 1.80 mol, 52.99% yield) as a yellow oil. ¹H NMR (400 MHz, chloroform-d) δ 7.29-7.15 (m, 5H), 4.42 (s, 2H), 3.51 (t, J=6.6 Hz, 2H), 3.38 (t, J=6.6 Hz, 2H), 1.86-1.69 (m, 1H), 1.59-1.22 (m, 10H).

### (vi) Preparation of Compound 8

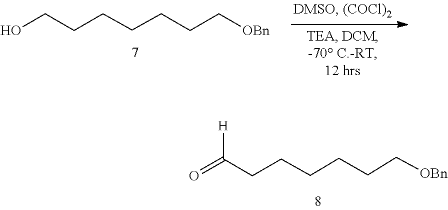 [SMILES: OCCCCCCCOCc1ccccc1 ; O=CCCCCCCOCc1ccccc1]

To a solution of (COCl)₂ (85.64 g, 674.70 mmol, 59.06 mL, 1.5 eq.) in DCM (400 mL) was added DMSO (87.86 g, 1.12 mol, 87.86 mL, 2.5 eq.) at −70° C. under N₂ atmosphere. The mixture was stirred at −70° C. for 0.5 hour. Then a solution of 7-benzyloxyheptan-1-ol (100 g, 449.80 mmol, 1.0 eq.) in DCM (1100 mL) was added to the mixture at −70° C. and the mixture was stirred at −70° C. for 0.5 hour, followed by the addition of TEA (250.33 g, 2.47 mol, 344.33 mL, 5.5 eq.). The mixture was stirred at −70° C. for 1 hour under N₂ atmosphere and then slow warm to 20° C. for 10 hours under N₂ atmosphere. The reaction mixture was quenched with sat. aq. NH₄Cl (3000 mL) and extracted with dichloromethane (3000 mL×3). The organic layer was dried over Na₂SO₄, filtered and concentrated under reduced pressure to give 7-benzyloxyheptanal (400 g, crude) as a yellow oil. ¹H NMR (400 MHz, chloroform-d) δ 9.76 (s, 1H), 7.39-7.24 (m, 5H), 4.51 (s, 2H), 3.48 (t, J=6.5 Hz, 2H), 2.43 (t, J=7.3 Hz, 2H), 1.64 (sxt, J=6.9 Hz, 4H), 1.47-1.31 (m, 4H).

(vii) Preparation of Compound 9.

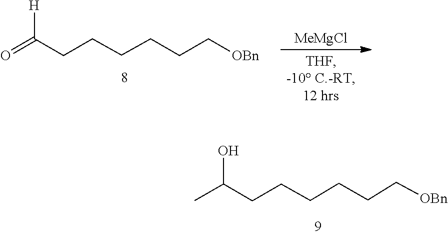 [SMILES: "MeMgCl" ; O=CCCCCCCOCc1ccccc1 ; CC(O)CCCCCCOCc1ccccc1]

To a solution of 7-benzyloxyheptanal (100 g, 453.91 mmol, 1.0 eq.) in THF (1000 mL) was added MeMgBr (3 M, 226.96 mL, 1.5 eq.) at −10° C. under N₂ atmosphere. The mixture was stirred at 20° C. for 12 hours under N₂ atmosphere. The reaction mixture was quenched with sat. aq. NH₄Cl (2000 mL) and extracted with ethyl acetate (2000 mL×3). The organic layer was washed with brine (150 mL), dried over Na₂SO₄, filtered and concentrated under reduced pressure to give a residue. The residue was purified by column chromatography (SiO₂, petroleum ether:ethyl acetate=100:1 to 3:1) to give 8-benzyloxyoctan-2-ol (120 g, 507.72 mmol, 27.96% yield) as a yellow oil. ¹H NMR (400 MHz, chloroform-d) δ 7.32-7.15 (m, 5H), 4.43 (s, 2H), 3.77-3.64 (m, 1H), 3.39 (t, J=6.6 Hz, 2H), 1.59-1.50 (m, 2H), 1.40-1.21 (m, 9H), 1.13-1.07 (m, 3H).

(viii) Preparation of Compound 10.

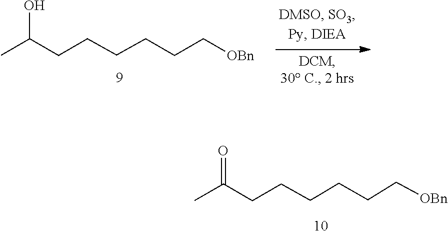 [SMILES: CC(O)CCCCCCOCc1ccccc1 ; CC(=O)CCCCCCOCc1ccccc1]

To a solution of 8-benzyloxyoctan-2-ol (60 g, 253.86 mmol, 1.0 eq.) in DCM (1000 mL) was added DIEA (131.24 g, 1.02 mol, 176.87 mL, 4.0 eq.), DMSO (79.34 g, 1.02 mol, 79.34 mL, 4.0 eq.), and then SO₃·Py (161.62 g, 1.02 mol, 4.0 eq.) was added at 0° C. The mixture was stirred at 30° C. for 2 hours. The reaction mixture was added H₂O (1000 mL) and extracted with dichloromethane (200 mL×3). The organic layer was washed with brine (50 mL), dried over Na₂SO₄, filtered and concentrated under reduced pressure to give a residue. The residue was purified by column chromatography (SiO₂, petroleum ether:ethyl acetate=100:1 to 3:1) to give 8-benzyloxyoctan-2-one (80 g, 341.39 mmol, 67.24% yield) as a yellow oil. ¹H NMR (400 MHz, chloroform-d) δ 7.41-7.26 (m, 5H), 4.52 (s, 2H), 3.48 (t, J=6.5 Hz, 2H), 2.43 (t, J=7.5 Hz, 2H), 2.15 (s, 3H), 1.62 (quind, J=7.2, 14.6 Hz, 4H), 1.46-1.28 (m, 4H).

### (ix) Preparation of Compound 11.

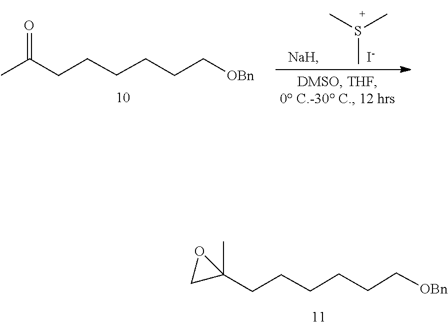

To a solution of NaH (16.64 g, 416.07 mmol, 60% purity, 1.5 eq.) in THF (1000 mL) was added trimethylsulfonium; iodide (73.59 g, 360.60 mmol, 1.3 eq.) in DMSO (1000 mL) at 0° C. Then 8-benzyloxyoctan-2-one (65 g, 277.38 mmol, 1.0 eq.) was added at 0° C. The mixture was stirred at 30° C. for 12 hours. The reaction mixture was quenched with sat. aq. NH₄Cl (1000 mL), concentrated to remove solvent and extracted with ethyl acetate (1000 mL×3). The organic layer was washed with brine (1000 mL), dried over Na₂SO₄, filtered and concentrated under reduced pressure to give a residue. The residue was purified by column chromatography (SiO₂, petroleum ether ethyl acetate=100:1 to 4:1) to give 2-(6-benzyloxyhexyl)-2-methyl-oxirane (51 g, 205.35 mmol, 74.03% yield) as a yellow oil. ¹H NMR (400 MHz, chloroform-d) δ 7.32-7.14 (m, 5H), 4.43 (s, 2H), 3.39 (t, J=6.6 Hz, 2H), 2.57-2.45 (m, 2H), 1.60-1.46 (m, 3H), 1.46-1.20 (m, 10H).

### (x) Preparation of Compound 12.

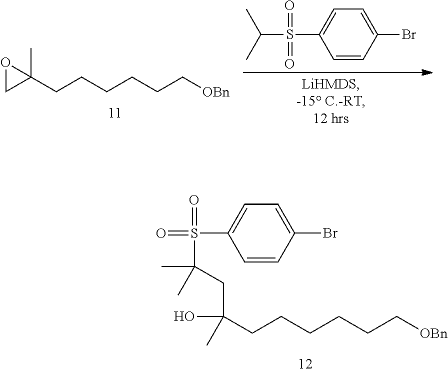 [SMILES: CC1(CCCCCCOCc2ccccc2)CO1 ; CC(C)S(=O)(=O)c1ccc(Br)cc1 ; CC(O)(CCCCCCOCc1ccccc1)CC(C)(C)S(=O)(=O)c1ccc(Br)cc1]

To a solution of 2-(6-benzyloxyhexyl)-2-methyl-oxirane (10 g, 40.26 mmol, 1.2 eq.) and 1-bromo-4-isopropylsulfonyl-benzene (8.83 g, 33.55 mmol, 1.0 eq.) in THF (80 mL) was added dropwise LiHMDS (1 M, 50.33 mL, 1.5 eq.) at −10° C. under N₂ atmosphere. The mixture was stirred at 20° C. for 12 hours. The reaction mixture was quenched with sat. aq. NH₄Cl (300 mL), concentrated to remove solvent, then the mixture was extracted with ethyl acetate (500 mL). The organic layer was washed with brine (100 mL), dried over Na₂SO₄, filtered and concentrated under reduced pressure to give a residue. The residue was purified by column chromatography (SiO₂, petroleum ether:ethyl acetate=100:1 to 5:1) to give 10-benzyloxy-2-(4-bromophenyl)sulfonyl-2,4-dimethyl-decan-4-ol (8 g, 15.64 mmol, 67.44% yield) as a yellow oil. ESI [M+Na⁺]=533.1/535.1.

### (xi) Preparation of Compound 13.

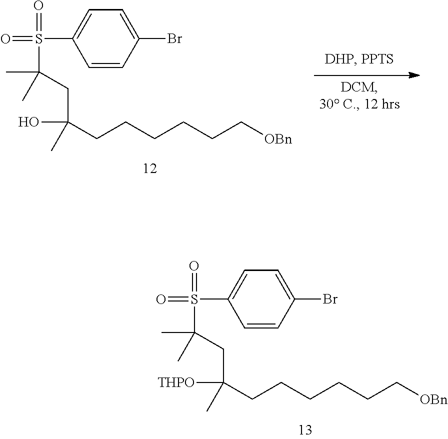

To a solution of 10-benzyloxy-2-(4-bromophenyl)sulfonyl-2,4-dimethyl-decan-4-ol (20 g, 39.10 mmol, 1.0 eq.) in DCM (300 mL) was added DHP (65.78 g, 782.00 mmol, 71.50 mL, 20.0 eq.) and PPTS (982.59 mg, 3.91 mmol, 0.1 eq.), the mixture was stirred at 30° C. for 12 hours under N₂ atmosphere. The reaction mixture was diluted with H₂O (300 mL) and extracted with dichloromethane (250 mL×2). The organic layer was dried over Na₂SO₄, filtered and concentrated under reduced pressure to give a residue. The residue was purified by column chromatography (SiO₂, petroleum ether:ethyl acetate=100:1 to 3:1) to give 2-[7-benzyloxy-1-[2-(4-bromophenyl)sulfonyl-2-methyl-propyl]-1-methyl-heptoxy]tetrahydropyran (20 g, 33.58 mmol, 85.88% yield) as a yellow oil. ESI [M+Na⁺]=617.2/619.2.

(xii) Preparation of Compound 14

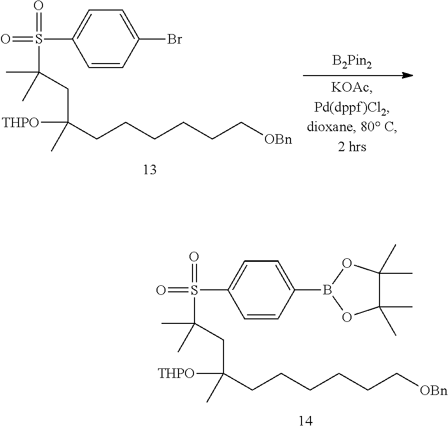

A mixture of 2-[7-benzyloxy-1-[2-(4-bromophenyl)sulfonyl-2-methyl-propyl]-1-methyl-heptoxy]tetrahydropyran (10 g, 16.79 mmol, 1.0 eq.), 4,4,5,5-tetramethyl-2-(4,4,5,5-tetramethyl-1,3,2-dioxaborolan-2-yl)-1,3,2-dioxaborolane (5.12 g, 20.15 mmol, 1.2 eq.), KOAc (4.94 g, 50.37 mmol, 3.0 eq.), Pd(dppf)Cl₂ (1.23 g, 1.68 mmol, 0.1 eq.) in dioxane (200 mL) was degassed and purged with N₂ for 3 times, and then the mixture was stirred at 80° C. for 2 hours under N₂ atmosphere. The reaction solution was used directly to next step. ESI [M+Na⁺]=665.3.

(xiii) Preparation of Compound 15

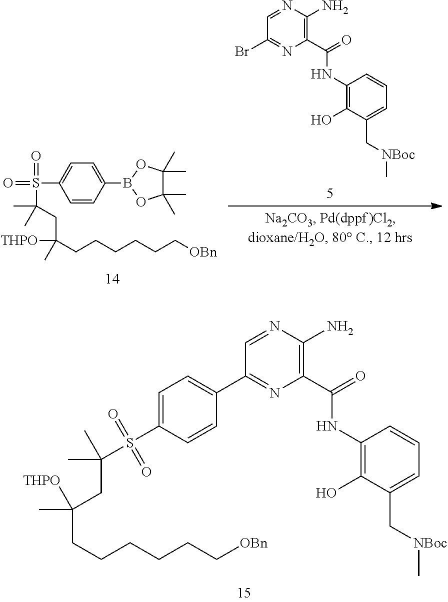

A mixture of 2-[4-(9-benzyloxy-1,1,3-trimethyl-3-tetrahydropyran-2-yloxy-nonyl)sulfonylphenyl]-4,4,5,5-tetramethyl-1,3,2-dioxaborolane (10.8 g, 16.80 mmol, 1.0 eq.), tert-butyl N-[[3-[(3-amino-6-bromo-pyrazine-2-carbonyl)amino]-2-hydroxyphenyl]methyl]-N-methyl-carbamate (7.60 g, 16.80 mmol, 1.0 eq.), Na₂CO₃ (2.67 g, 25.21 mmol, 1.5 eq.), Pd(dppf)Cl₂ (1.23 g, 1.68 mmol, 0.1 eq.) in dioxane (250 mL) and H₂O (50 mL) was degassed and purged with N₂ for 3 times, and then the mixture was stirred at 80° C. for 12 hours under N₂ atmosphere. The reaction mixture was concentrated to remove solvent, then added H₂O (200 mL) and extracted with ethyl acetate (300 mL). The organic layer was washed with brine (200 mL), dried over Na₂SO₄, filtered and concentrated under reduced pressure to give a residue. The residue was purified by column chromatography (SiO₂, petroleum ether:ethyl acetate=100:1 to 1:1) to give tert-butyl N-[[3-[[3-amino-6-[4-(9-benzyloxy-1,1,3-trimethyl-3-tetrahydropyran-2-yloxynonyl)sulfonylphenyl]pyrazine-2-carbonyl]amino]-2-hydroxy-phenyl]methyl]-N-methyl-carbamate (27.6 g, 31.08 mmol, 92.47% yield) as a yellow oil. ESI [M+Na⁺]=910.4.

(xvi) Preparation of Compound 16

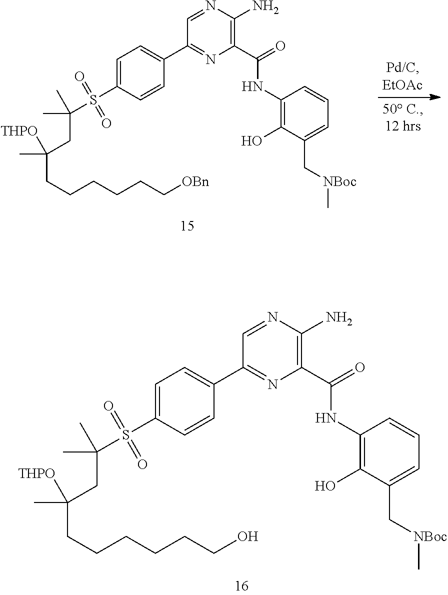

A mixture of tert-butyl N-[[3-[[3-amino-6-[4-(9-benzyloxy-1,1,3-trimethyl-3-tetrahydropyran-2-yloxynonyl)sulfonylphenyl]pyrazine-2-carbonyl]amino]-2-hydroxy-phenyl]methyl]-N-methyl-carbamate (6 g, 6.76 mmol, 1.0 eq.), Pd/C (2.6 g, 10% purity) in EtOAc (90 mL) was degassed and purged with H₂ for 3 times, and then the mixture was stirred at 50° C. for 12 hours under H₂ (50 Psi) atmosphere. The reaction mixture was filtered, the filtrate was concentrated under reduced pressure to give a residue. The residue was purified by column chromatography (SiO₂, petroleum ether:tetrahydrofuran=100:1 to 1:1) to give tert-butyl N-[[3-[[3-amino-6-[4-(9-hydroxy-1,1,3-trimethyl-3-tetrahydropyran-2-yloxynonyl)sulfonylphenyl]pyrazine-2-carbonyl]amino]-2-hydroxy-phenyl]methyl]-N-methyl-carbamate (4.5 g, 5.64 mmol, 41.74% yield) as a yellow oil. ESI [M+Na⁺]=820.4.

### (xv) Preparation of Compound 17.

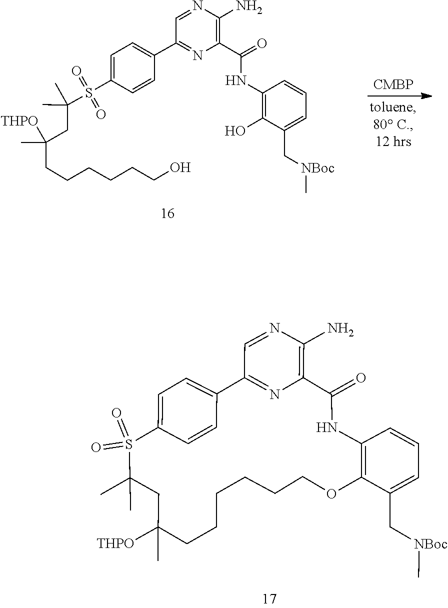

A mixture of tert-butyl N-[[3-[[3-amino-6-[4-(9-hydroxy-1,1,3-trimethyl-3-tetrahydropyran-2-yloxynonyl)sulfonylphenyl]pyrazine-2-carbonyl]amino]-2-hydroxy-phenyl]methyl]-N-methyl-carbamate (4.2 g, 5.26 mmol, 1.0 eq.), 2-(tributyl-λ⁵-phosphanylidene)acetonitrile (3.81 g, 15.79 mmol, 3.0 eq.) in Tol. (80 mL) was degassed and purged with N₂ for 3 times, and then the mixture was stirred at 80° C. for 12 hours under N₂ atmosphere. The reaction mixture was diluted with H₂O (100 mL) and extracted with Ethyl acetate (100 mL×2). The organic layer was washed with brine (100 mL), dried over Na₂SO₄, filtered and concentrated under reduced pressure to give a residue. The residue was purified by column chromatography (SiO₂, petroleum ether ethyl acetate=100:1 to 1:1) to give tert-butyl N-[(5-amino-22,24,24-trimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (2.6 g, 3.33 mmol, 63.33% yield) as a yellow oil. ESI [M+Na⁺]=802.4.

(xvi) Preparation of Compound A

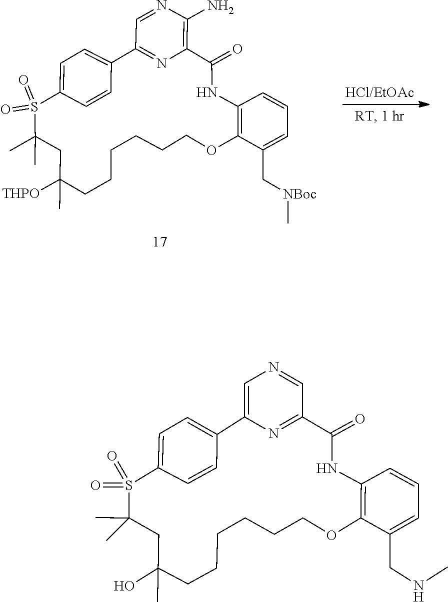

A solution of tert-butyl N-[(5-amino-22,24,24-trimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (1.9 g, 2.44 mmol, 1.0 eq.) in HCl/EtOAc (25 mL; 4 M) was stirred at 25° C. for 1 hour. The reaction mixture was concentrated under reduced pressure to give a residue. The residue was triturated with EtOH (20 mL) to give 5-amino-22-hydroxy-22,24,24-trimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (1.34 g, 2.08 mmol, 85.21% yield, 97.93% purity, HCl) as a yellow solid. ¹H NMR (400 MHz, DMSO-d₆) δ 10.40 (s, 1H), 8.95-9.14 (m, 3H), 8.46 (dd, J=1.63, 7.88 Hz, 1H), 8.37 (d, J=8.63 Hz, 2H), 7.89-8.07 (m, 4H), 7.30-7.40 (m, 2H), 4.20 (br t, J=5.69 Hz, 2H), 3.83-3.94 (m, 2H), 2.64 (br t, J=5.19 Hz, 3H), 1.73-1.83 (m, 3H), 1.54 (s, 3H), 1.48 (s, 3H), 1.29-1.40 (m, 5H), 1.11-1.22 (m, 5H), 0.98 (br d, J=6.13 Hz, 2H). ESI [M+H]=596.2.

### Example 2: Preparation of Compound B

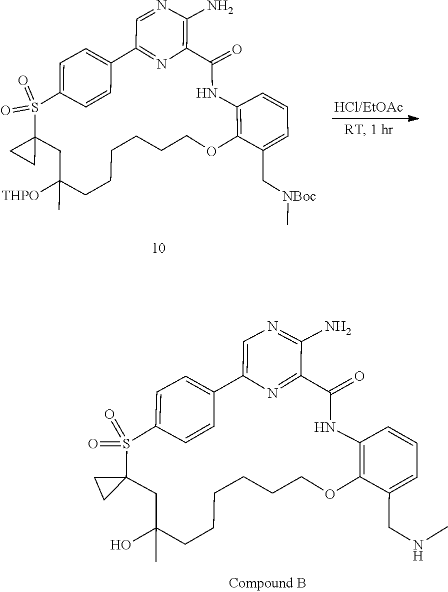

A mixture of tert-butyl N-[(5-amino-22-methyl-7,25,25-trioxo-22-tetrahydropyran-2-yloxy-spiro[15-oxa-25λ⁶-thia-4,8,31-triazatetracyclo[24.2.2.1^{2,6}.0^{9,14}]hentriaconta-1(28),2(31),3,5,9(14),10,12,26,29-nonaene-24,1′-cyclopropane]-13-yl)methyl]-Nmethyl-carbamate (compound 10; 2 g, 2.57 mmol, 1.0 eq.) in HCl/EtOAc (5 mL; 4 M) was stirred at 25° C. for 1 hour. The mixture was concentrated in vacuo and the residue was purified by prep-HPLC (column: Phenomenex luna C18 250*50 mm*10 μm; mobile phase: [water(HCl)-ACN]; B %: 20%-50%,10 min) to give 5-amino-22-hydroxy-22-methyl-13-(methylaminomethyl)-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (1 g, 1.59 mmol, 61.72% yield, HCl) as a yellow solid. ¹H NMR (400 MHz, DMSO-d₆) δ 10.33 (s, 1H), 9.29-9.00 (m, 3H), 8.43-8.24 (m, 3H), 7.98 (br d, J=8.5 Hz, 3H), 7.44 (dd, J=1.3, 7.8 Hz, 1H), 7.36-7.27 (m, 1H), 4.19 (br t, J=5.7 Hz, 2H), 3.97-3.72 (m, 2H), 2.63 (br t, J=5.3 Hz, 3H), 2.10-2.00 (m, 1H), 1.99-1.89 (m, 1H), 1.74 (tt, J=6.1, 11.9 Hz, 1H), 1.68-1.54 (m, 3H), 1.54-1.43 (m, 2H), 1.41-1.30 (m, 1H), 1.21 (qd, J=5.6, 11.6 Hz, 1H), 1.15-1.00 (m, 2H), 0.99-0.64 (m, 7H). ESI [M+H]=594.4.

### Example 3: Preparation of Compound C

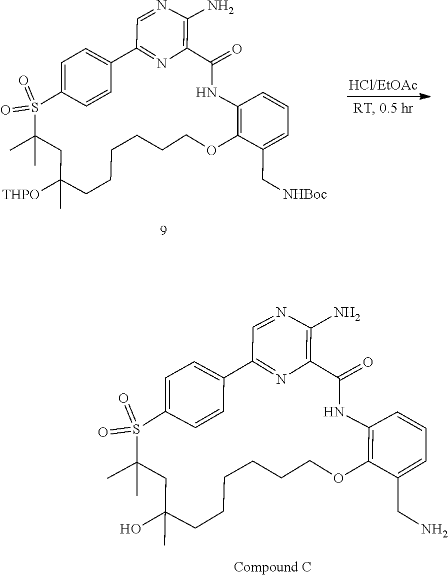

A solution of tert-butyl N-[(5-amino-22,24,24-trimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (65 mg, 84.86 μmol, 1.0 eq.) in HCl/EtOAc (2 mL; 4 M) was stirred at 25° C. for 0.5 hour. The reaction mixture was concentrated under reduced pressure to give a residue. The residue was purified by prep-HPLC (column: Phenomenex luna C18 80*40 mm*3 μm; mobile phase: [water (0.04% HCl)-ACN]; B %: 22%-40%, 7 min) to give 5-amino-13-(aminomethyl)-22-hydroxy-22,24,24-trimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (50 mg, 77.95 μmol, 91.86% yield, 96.38% purity, HCl) as a yellow solid. ¹H NMR (400 MHz, DMSO-d₆) δ 10.40 (s, 1H), 9.13 (s, 1H), 8.48-8.24 (m, 6H), 8.09-7.89 (m, 4H), 7.32 (d, J=4.8 Hz, 2H), 4.13 (br d, J=5.9 Hz, 2H), 3.95-3.83 (m, 2H), 1.77 (br d, J=14.1 Hz, 3H), 1.54 (s, 3H), 1.49 (s, 3H), 1.45-1.29 (m, 5H), 1.23 (br d, J=6.3 Hz, 1H), 1.16 (s, 4H), 1.01 (br s, 2H). ESI [M+H]=582.3.

### Example 4: Preparation of Compound D

Compound D was prepared using the general procedures for Examples 1-3 and 5-19.

### Example 5: Preparation of Compound E

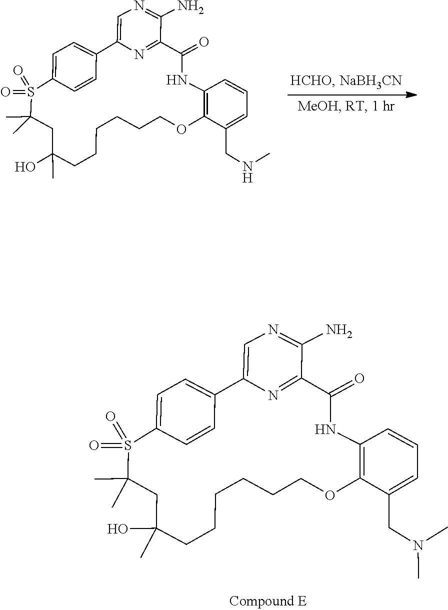 [SMILES: CNCc1cccc2c1OCCCCCCC(C)(O)CC(C)(C)S(=O)(=O)c1ccc(cc1)-c1cnc(N)c(n1)C(=O)N2 ; CN(C)Cc1cccc2c1OCCCCCCC(C)(O)CC(C)(C)S(=O)(=O)c1ccc(cc1)-c1cnc(N)c(n1)C(=O)N2]

To a solution of 5-amino-22-hydroxy-22,24,24-trimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-7-one (50 mg, 79.09 μmol, 1.0 eq., HCl) in MeOH (2 mL) was added DIEA (30.66 mg, 237.26 μmol, 41.33 μL, 3.0 eq.), AcOH (4.75 mg, 79.09 μmol, 4.52 μL, 1.0 eq.) and HCHO (12.84 mg, 158.17 μmol, 11.78 μL, 37% purity, 2.0 eq.). The mixture was stirred at 25° C. for 0.5 hour. Then NaBH₃CN (4.97 mg, 79.09 μmol, 1.0 eq.) was added. The mixture was stirred at 25° C. for 0.5 hour. The mixture was added sat. aq. Na₂CO₃(3 mL) and extracted with dichloromethane (5 mL×2). The organic layers were dried over Na₂SO₄, filtered and concentrated under reduced pressure to give a residue. The residue was purified by prep-HPLC (column: Phenomenex Luna 80*30 mm*3 μm; mobile phase: [water(HCl)-ACN]; B %: 10%-40%, 8 min) to give 5-amino-13-[(dimethylamino)methyl]-22-hydroxy-22,24,24-trimethyl-25,25-dioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-7-one (30.91 mg, 47.83 μmol, 60.48% yield, 100% purity, HCl) as a yellow solid. ¹H NMR (400 MHz, DMSO-d₆) δ 10.42-10.29 (m, 2H), 9.24-9.07 (m, 2H), 8.51-8.43 (m, 1H), 8.40-8.35 (m, 2H), 7.92 (br d, J=8.4 Hz, 4H), 7.51-7.40 (m, 1H), 7.34 (t, J=8.1 Hz, 1H), 4.32 (br d, J=5.1 Hz, 2H), 3.90-3.81 (m, 2H), 2.77 (br t, J=5.9 Hz, 6H), 1.84-1.73 (m, 3H), 1.59 (br d, J=14.1 Hz, 1H), 1.53 (s, 3H), 1.48 (s, 3H), 1.33 (br t, J=7.5 Hz, 4H), 1.24-1.14 (m, 5H), 1.03-0.93 (m, 2H). ESI [M+H]=610.2

### Example 6: Preparation of Compound F

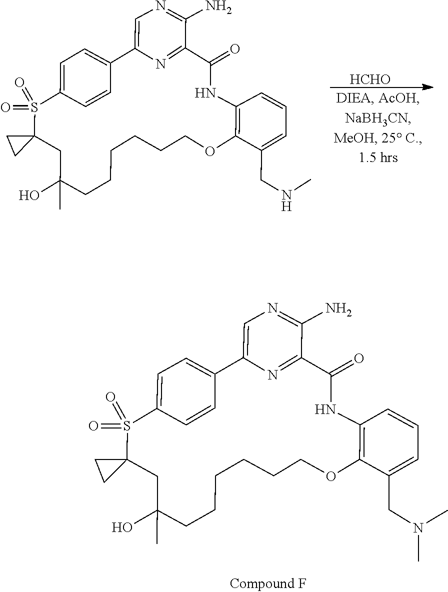 [SMILES: CNCc1cccc2c1OCCCCCCC(C)(O)CC1(CC1)S(=O)(=O)c1ccc(cc1)-c1cnc(N)c(n1)C(=O)N2 ; "HCHO" ; CN(C)Cc1cccc2c1OCCCCCCC(C)(O)CC1(CC1)S(=O)(=O)c1ccc(cc1)-c1cnc(N)c(n1)C(=O)N2]

To a solution of 5-amino-22-hydroxy-22-methyl-13-(methylaminomethyl)-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (61 mg, 96.80 μmol, 1.0 eq., HCl) in MeOH (2 mL) was added DIEA (37.53 mg, 290.39 μmol, 50.58 μL, 3.0 eq.), AcOH (5.81 mg, 96.80 μmol, 5.54 μL, 1.0 eq.) and HCHO (15.71 mg, 193.59 μmol, 14.41 μL, 37% purity, 2.0 eq.). The mixture was stirred at 25° C. for 0.5 hour. Then NaBH₃CN (6.08 mg, 96.80 μmol, 1.0 eq.) was added. The mixture was stirred at 25° C. for 1 hour. The mixture was added sat. aq. Na₂CO₃ (3 mL) and extracted with dichloromethane (5 mL×2). The organic layer was dried over Na₂SO₄, filtered and concentrated to give a residue. Then the residue was purified by prep-HPLC (column: Phenomenex Luna 80*30 mm*3 μm; mobile phase: [water(HCl)-ACN]; B %: 10%-40%,8 min) to give 5-amino-13-[(dimethylamino)methyl]-22-hydroxy-22-methyl-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (46.45 mg, 72.10 μmol, 74.49% yield, 100% purity, HCl) as a yellow solid. ¹H NMR (400 MHz, DMSO-d₆) δ 10.34 (s, 1H), 10.12 (br s, 1H), 9.09 (s, 1H), 8.42-8.27 (m, 3H), 7.98 (br d, J=7.9 Hz, 4H), 7.52-7.39 (m, 1H), 7.34 (br t, J=7.8 Hz, 1H), 4.34 (br s, 2H), 3.88-3.77 (m, 2H), 2.79 (br s, 6H), 2.09-2.01 (m, 1H), 1.98-1.89 (m, 1H), 1.74 (br d, J=8.3 Hz, 1H), 1.68-1.56 (m, 3H), 1.53-1.44 (m, 2H), 1.34 (br d, J=3.8 Hz, 1H), 1.22 (br dd, J=4.8, 11.5 Hz, 1H), 1.09 (br d, J=9.0 Hz, 1H), 0.98-0.68 (m, 8H). ESI [M+H]=608.4.

### Example 7: Preparation of Compound H-2

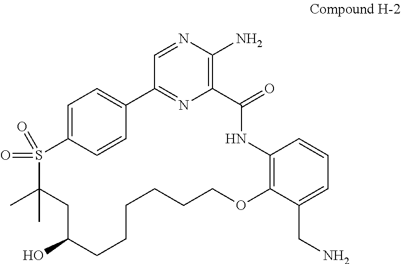 [SMILES: CC1(C)C[C@H](O)CCCCCCOc2c(CN)cccc2NC(=O)c2nc(cnc2N)-c2ccc(cc2)S1(=O)=O]

### (i) Preparation of Compound 2

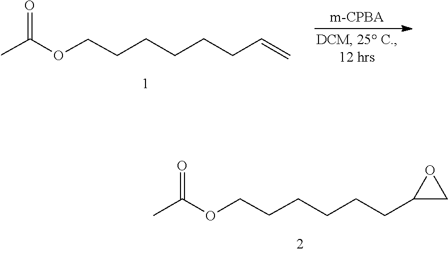

To a solution of oct-7-enyl acetate (9.4 g, 55.21 mmol, 1.0 eq.) in DCM (500 mL) was added m-CPBA (33.63 g, 165.64 mmol, 85% purity, 3.0 eq.) at 0° C. The mixture was stirred at 25° C. for 12 hours. The reaction mixture was quenched with sat. aq. Na₂SO₃ (500 mL) and extracted with dichloromethane (800 mL×2). The organic layer was washed with sat. aq. NaHCO₃ (300 mL×2), dried over Na₂SO₄, filtered and concentrated to give 6-(oxiran-2-yl)hexyl acetate (10.2 g, crude) as a yellow oil. ¹H NMR (400 MHz, chloroform-d) δ 3.99 (t, J=6.7 Hz, 2H), 2.87-2.81 (m, 1H), 2.68 (t, J=4.4 Hz, 1H), 2.40 (dd, J=2.7, 4.8 Hz, 1H), 1.98 (s, 3H), 1.60-1.52 (m, 2H), 1.51-1.37 (m, 4H), 1.31 (br d, J=3.6 Hz, 4H).

### (ii) Preparation of Compound 3

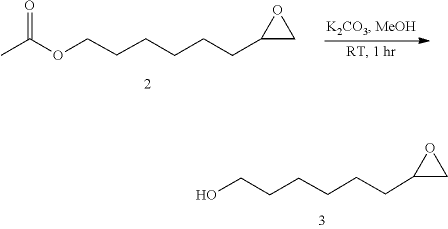

To a solution of 6-(oxiran-2-yl)hexyl acetate (10.2 g, 54.77 mmol, 1.0 eq.) in MeOH (150 mL) was added K₂CO₃ (11.35 g, 82.15 mmol, 1.5 eq.). The mixture was stirred at 25° C. for 1 hour. The mixture was filtered and concentrated to give a residue. The residue was diluted with H₂O (100 mL) and extracted with dichloromethane:methanol=8:1 (300 mL×2). The organic layer was dried over Na₂SO₄, filtered and concentrated to give 6-(oxiran-2-yl)hexan-1-ol (7.9 g, crude) as a yellow oil. ¹H NMR (400 MHz, chloroform-d) δ 3.58 (t, J=6.5 Hz, 2H), 2.88-2.79 (m, 1H), 2.68 (t, J=4.5 Hz, 1H), 2.40 (dd, J=2.8, 4.8 Hz, 1H), 1.56-1.31 (m, 10H).

(iii) Preparation of Compound 4

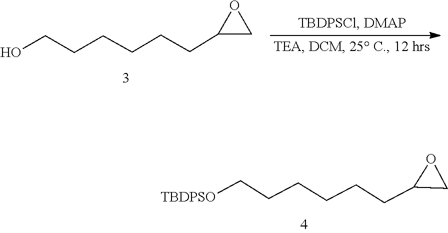

To a solution of 6-(oxiran-2-yl)hexan-1-ol (7.9 g, 54.78 mmol, 1.0 eq.) in DCM (100 mL) was added TEA (16.63 g, 164.34 mmol, 22.87 mL, 3.0 eq.) and DMAP (669.25 mg, 5.48 mmol, 0.1 eq.). Then TBDPSCl (18.07 g, 65.74 mmol, 16.89 mL, 1.2 eq.) was added at 0° C. The mixture was stirred at 25° C. for 12 hours. The reaction mixture was diluted with H₂O (50 mL) and extracted with dichloromethane (100 mL×2). The organic layer was dried over Na₂SO₄, filtered and concentrated to give a residue. Then the residue was purified by column chromatography (SiO₂, petroleum ether:ethyl acetate=100:1 to 0:1) to give tert-butyl-[6-(oxiran-2-yl)hexoxy]-diphenyl-silane (14.9 g, 38.94 mmol, 71.09% yield) as a yellow oil. ¹H NMR (400 MHz, chloroform-d) δ 7.59 (dd, J=1.5, 7.8 Hz, 4H), 7.38-7.27 (m, 6H), 3.58 (t, J=6.4 Hz, 2H), 2.85-2.79 (m, 1H), 2.67 (t, J=4.5 Hz, 1H), 2.38 (dd, J=2.8, 5.0 Hz, 1H), 1.52-1.26 (m, 10H), 0.97 (s, 9H).

### (iv) Preparation of Compound 5

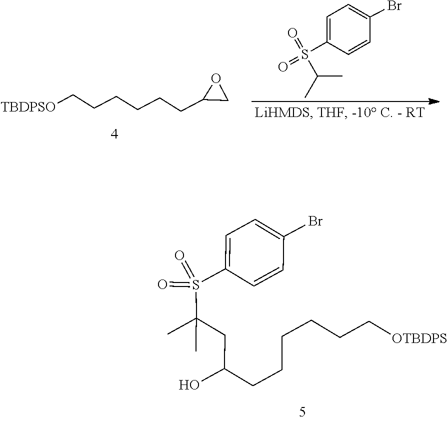

A mixture of tert-butyl-[6-(oxiran-2-yl)hexoxy]-diphenyl-silane (6.08 g, 15.88 mmol, 1.1 eq.) and 1-bromo-4-isopropylsulfonylbenzene (3.8 g, 14.44 mmol, 1.0 eq.) in THF (80 mL) was added dropwise LiHMDS (1 M, 21.66 mL, 1.5 eq.) at −10° C. The mixture was stirred at 15° C. for 5 hours under N₂ atmosphere. The mixture was quenched with sat. aq. NH₄Cl (60 mL) at 0° C. and extracted with ethyl acetate (100 ml×2). The organic layer was dried over Na₂SO₄, filtered and concentrated to give a residue. Then the residue was purified by column chromatography (SiO₂, petroleum ether ethyl acetate=100:1 to 0:1) to give 2-(4-bromophenyl)sulfonyl-10-[tert-butyl(diphenyl)silyl]oxy-2-methyl-decan-4-ol (14.1 g, 21.83 mmol, 75.60% yield) as a yellow oil. ¹H NMR (400 MHz, chloroform-d) δ 7.78-7.68 (m, 8H), 7.48-7.38 (m, 6H), 3.90 (br d, J=1.2 Hz, 1H), 3.68 (br t, J=6.3 Hz, 2H), 1.59 (br d, J=2.4 Hz, 2H), 1.48-1.28 (m, 16H), 1.08 (s, 9H).

### (v) Preparation of Compound 6

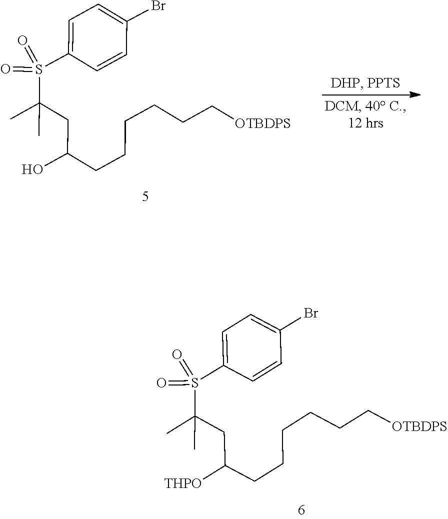

To a solution of 2-(4-bromophenyl)sulfonyl-10-[tert-butyl(diphenyl)silyl]oxy-2-methyl-decan-4-ol (14.1 g, 21.83 mmol, 1.0 eq.) in DCM (200 mL) was added DHP (45.92 g, 545.87 mmol, 49.91 mL, 25.0 eq.) and PPTS (548.71 mg, 2.18 mmol, 0.1 eq.). The mixture was stirred at 40° C. for 12 hours under N₂ atmosphere. The mixture was added H₂O (100 mL) and extracted with dichloromethane (200 mL×2). The organic layer was dried over Na₂SO₄, filtered and concentrated to give a residue. Then the residue was purified by column chromatography (SiO₂, petroleum ether:ethyl acetate=100:1 to 0:1) to give [9-(4-bromophenyl)sulfonyl-9-methyl-7-tetrahydropyran-2-yloxy-decoxy]-tert-butyl-diphenyl-silane (13.5 g, 18.50 mmol, 84.71% yield) as a yellow oil. ¹H NMR (400 MHz, chloroform-d) δ 7.77-7.66 (m, 8H), 7.48-7.37 (m, 6H), 4.62-4.57 (m, 1H), 3.95-3.84 (m, 2H), 3.79-3.71 (m, 1H), 3.68 (dt, J=2.8, 6.4 Hz, 2H), 1.62-1.45 (m, 13H), 1.41-1.23 (m, 11H), 1.07 (d, J=2.4 Hz, 9H). ESI [M+Na⁺]=753.2.

### (vi) Preparation of Compound 7

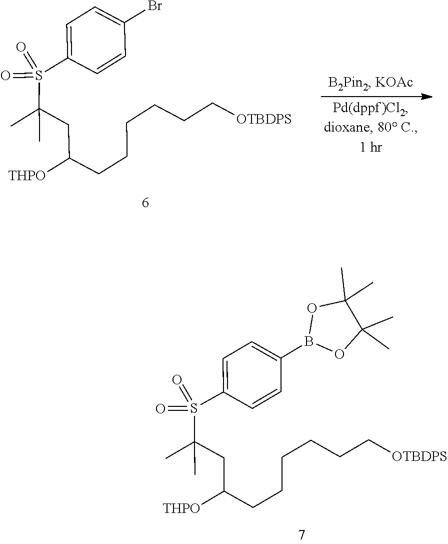

A mixture of [9-(4-bromophenyl)sulfonyl-9-methyl-7-tetrahydropyran-2-yloxy-decoxy]-tert-butyl-diphenyl-silane (2 g, 2.74 mmol, 1.0 eq.), 4,4,5,5-tetramethyl-2-(4,4,5,5-tetramethyl-1,3,2-dioxaborolan-2-yl)-1,3,2-dioxaborolane (765.42 mg, 3.01 mmol, 1.1 eq.), Pd(dppf)Cl₂(200.50 mg, 274.02 μmol, 0.1 eq.) and KOAc (537.86 mg, 5.48 mmol, 2.0 eq.) in dioxane (30 mL) was degassed and purged with N₂ for 3 times, and then the mixture was stirred at 80° C. for 1 hour under N₂ atmosphere. The reaction solution was used directly to next step. ESI [M+Na⁺]=799.4.

(vii) Preparation of Compound 8

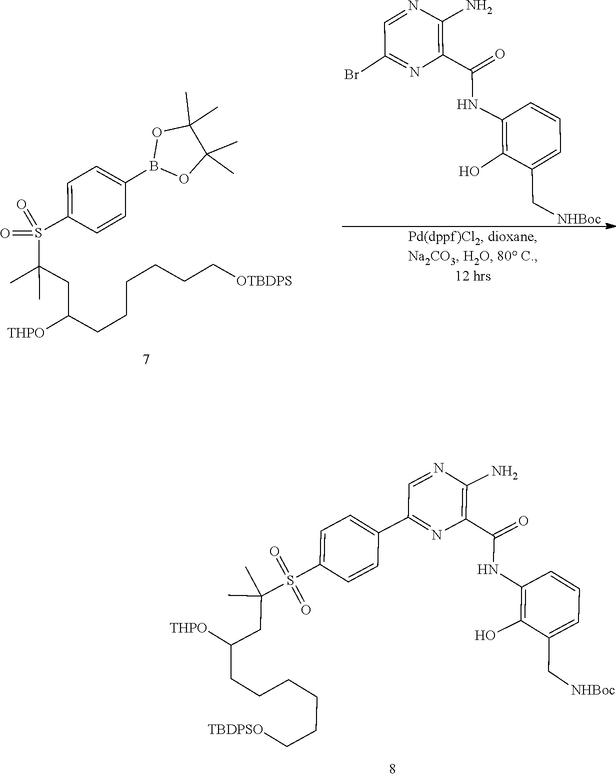

A mixture of tert-butyl-[9-methyl-7-tetrahydropyran-2-yloxy-9-[4-(4,4,5,5-tetramethyl-1,3,2-dioxaborolan-2-yl)phenyl]sulfonyldecoxy]-diphenyl-silane (2.1 g, 2.70 mmol, 1.0 eq.), tert-butylN-[[3-[(3-amino-6-bromo-pyrazine-2-carbonyl)amino]-2-hydroxy-phenyl]methyl]carbamate (1.07 g, 2.43 mmol, 0.9 eq.), Na₂CO₃ (572.96 mg, 5.41 mmol, 2.0 eq.) and Pd(PPh₃)₄ (624.67 mg, 540.58 μmol, 0.2 eq.) in dioxane (30 mL) and H₂O (6 mL) was degassed and purged with N₂ for 3 times, and then the mixture was stirred at 80° C. for 12 hours under N₂ atmosphere. The mixture was filtered and concentrated, and then diluted with H₂O (50 mL) and extracted with ethyl acetate (100 mL×2). The combined organic layers were dried over Na₂SO₄, filtered and concentrated to give a residue. Then the residue was purified by column chromatography (SiO₂, petroleum ether:ethyl acetate=100:1 to 0:1) to give tert-butyl N-[[3-[[3-amino-6-[4-[9-[tert-butyl(diphenyl)silyl]oxy-1,1-dimethyl-3-tetrahydropyran-2-yloxynonyl]sulfonylphenyl]pyrazine-2-carbonyl]amino]-2-hydroxy-phenyl]methyl]carbamate (2.3 g, 2.28 mmol, 84.39% yield) as a yellow solid. ¹H NMR (400 MHz, chloroform-d) δ 10.61 (br s, 1H), 9.77 (br s, 1H), 8.69 (d, J=3.5 Hz, 1H), 8.27 (br s, 1H), 8.13-8.06 (m, 2H), 7.92 (d, J=8.1 Hz, 2H), 7.59 (br d, J=7.4 Hz, 4H), 7.38-7.27 (m, 6H), 6.88-6.80 (m, 2H), 4.57-4.39 (m, 1H), 4.20 (d, J=6.6 Hz, 2H), 3.92-3.66 (m, 2H), 3.57 (dt, J=3.0, 6.4 Hz, 2H), 3.45-3.28 (m, 1H), 2.03-1.93 (m, 2H), 1.89-1.77 (m, 1H), 1.75-1.54 (m, 3H), 1.53-1.39 (m, 14H), 1.35-1.16 (m, 13H), 0.97 (d, J=2.6 Hz, 9H). ESI [M+H−THP−Boc]=824.4.

(viii) Preparation of Compound 9

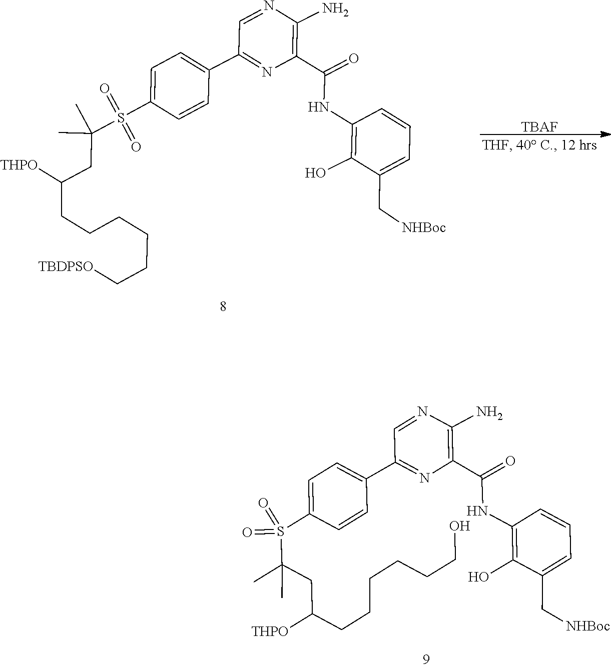

To a solution of tert-butyl N-[[3-[[3-amino-6-[4-[9-[tert-butyl(diphenyl)silyl]oxy-1,1-dimethyl-3-tetrahydropyran-2-yloxynonyl]sulfonylphenyl]pyrazine-2carbonyl]amino]-2-hydroxy-phenyl]methyl]carbamate (2.2 g, 2.18 mmol, 1.0 eq.) in THF (30 mL) was added TBAF (1 M, 4.36 mL, 2.0 eq.). The mixture was stirred at 40° C. for 12 hours. The mixture was concentrated in vacuo and the residue was diluted with ethyl acetate (100 mL) and washed with H₂O (30 mL*3). Then the combined organic layers were dried over Na₂SO₄, filtered and concentrated to give a residue. The residue was purified by column chromatography (SiO₂, petroleum ether:tetrahydrofuran=100:1 to 0:1) to give tert-butyl N-[[3-[[3-amino-6-[4-(9-hydroxy-1,1-dimethyl-3-tetrahydropyran-2-yloxy-nonyl)sulfonylphenyl]pyrazine-2-carbonyl]amino]-2-hydroxyphenyl]methyl]carbamate (760 mg, 987.08 μmol, 45.24% yield) as a yellow gum. ESI [M+H−THP−Boc]=586.3.

### (ix) Preparation of Compound 10

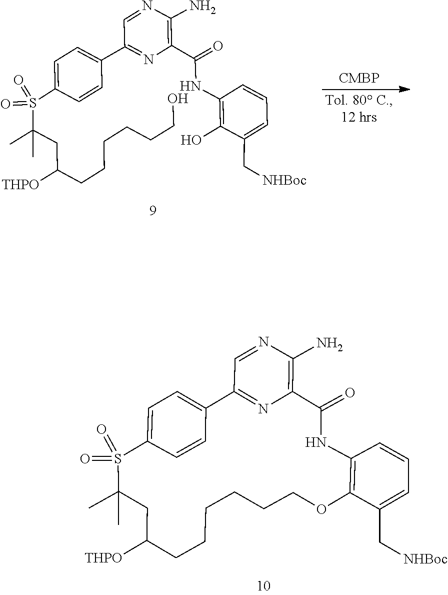

To a solution of tert-butyl N-[[3-[[3-amino-6-[4-(9-hydroxy-1,1-dimethyl-3-tetrahydropyran-2-yloxynonyl)sulfonylphenyl]pyrazine-2-carbonyl]amino]-2-hydroxy-phenyl]methyl]carbamate (750 mg, 974.10 μmol, 1.0 eq.) in Tol. (80 mL) was added a solution of 2-(tributyl-3-phosphanylidene)acetonitrile (705.30 mg, 2.92 mmol, 3.0 eq.) in Tol. (3 mL) at 0° C. The mixture was stirred at 80° C. for 12 hours. The mixture was concentrated and the residue was purified by column chromatography (SiO₂, petroleum ether:tetrahydrofuran=100:1 to 1:1) to give tert-butyl N-[(5-amino-24,24-dimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29 nonaen-13-yl)methyl]carbamate (800 mg, crude) as a yellow oil. ESI [M+H−THP−Boc]=568.3.

### (x) Preparation of Compound 11

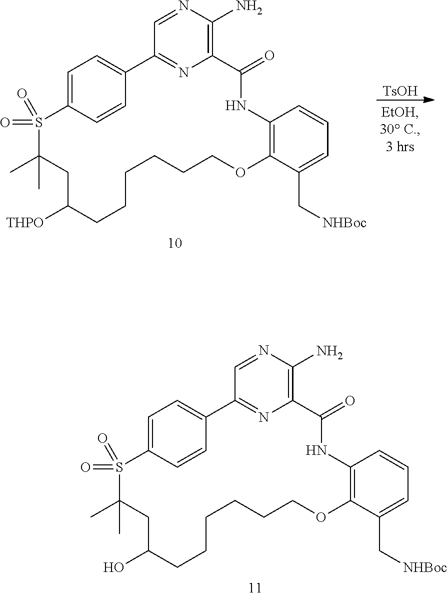

To a solution of tert-butyl N-[(5-amino-24,24-dimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (700 mg, 930.94 μmol, 1.0 eq.) in EtOH (10 mL) was added 4-methylbenzenesulfonic acid; hydrate (177.08 mg, 930.94 μmol, 1.0 eq.). The mixture was stirred at 30° C. for 3 hours. The mixture was quenched with sat. aq. Na₂CO₃ (10 mL) at 0° C. and extracted with Ethyl acetate (30 mL×3). The organic layer was dried over Na₂SO₄, filtered and concentrated to give a residue. Then the residue was purified by column chromatography (SiO₂, petroleum ether:tetrahydrofuran=100:1 to 0:1) to give tert-butyl N-[(5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (440 mg, 658.87 μmol, 70.77% yield) as a yellow solid. ESI [M+H−Boc]=568.2.

### (xi) Preparation of Compounds 12A and 12B

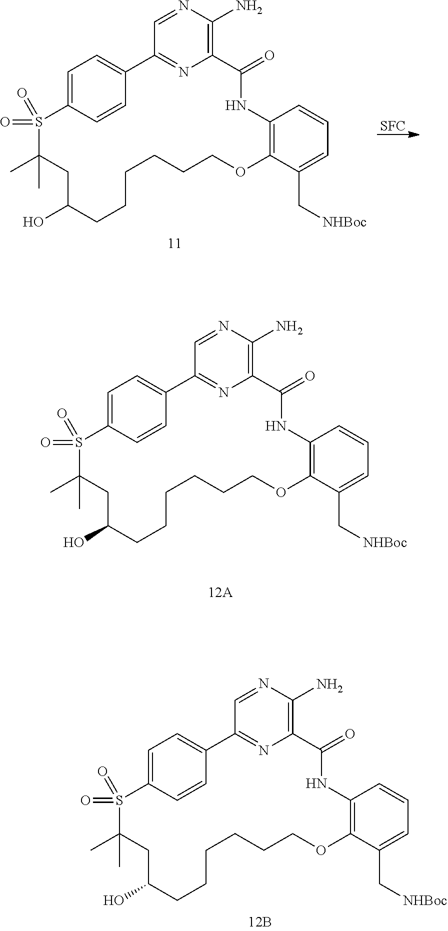

The racemic material was purified by SFC (column: DAICEL CHIRALPAKAD (250 mm*30 mm, 10 μm); mobile phase: [0.1% NH₃H₂O EtOH]; B %: 46%-46%, 8 min) to give arbitrarily assigned: tert-butyl N-[[(22R)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (Peak 1, retention time=1.813 min) (70 mg, 104.82 μmol, 35.00% yield, ee %=100%) as a yellow solid and tert-butyl N-[[(22S)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (Peak 2, retention time=2.038 min) (80 mg, 119.79 μmol, 40.00% yield, ee %=96.02%) as a yellow solid. ESI [M+H−Boc]=568.2.

(xii) Preparation of Compound H-2

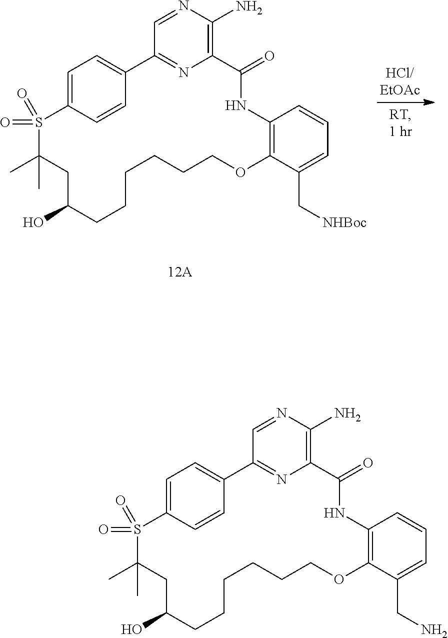 [SMILES: CC(C)(C)OC(=O)NCc1cccc2c1OCCCCCC[C@@H](O)CC(C)(C)S(=O)(=O)c1ccc(cc1)-c1cnc(N)c(n1)C(=O)N2 ; CC1(C)C[C@H](O)CCCCCCOc2c(CN)cccc2NC(=O)c2nc(cnc2N)-c2ccc(cc2)S1(=O)=O]

A mixture of tert-butyl N-[[(22R)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (70 mg, 104.82 μmol, 1.0 eq.) in HCl/EtOAc (2 mL; 4 M) was stirred at 20° C. for 1 hour. The mixture was concentrated and the residue purified by prep-HPLC (column: Phenomenex Luna 80*30 mm*3 μm; mobile phase: [water(HCl)-ACN]; B %: 20%-70%, 8 min) to give (22R)-5-amino-13-(aminomethyl)-22-hydroxy-24,24-dimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (46.27 mg, 76.59 μmol, 73.06% yield, 100% purity, HCl) as a yellow solid. ¹H NMR (400 MHz, DMSO-d₆) δ 10.40 (s, 1H), 9.13 (s, 1H), 8.49-8.30 (m, 6H), 8.15-7.84 (m, 4H), 7.37-7.27 (m, 2H), 4.12 (q, J=5.5 Hz, 2H), 3.94-3.88 (m, 2H), 3.60-3.53 (m, 1H), 1.78 (quin, J=7.3 Hz, 2H), 1.68 (br d, J=13.9 Hz, 1H), 1.52-1.38 (m, 7H), 1.34 (s, 4H), 1.26-1.16 (m, 2H), 1.15-1.06 (m, 2H). ESI [M+H]=568.4

### Example 8: Preparation of Compound H-1

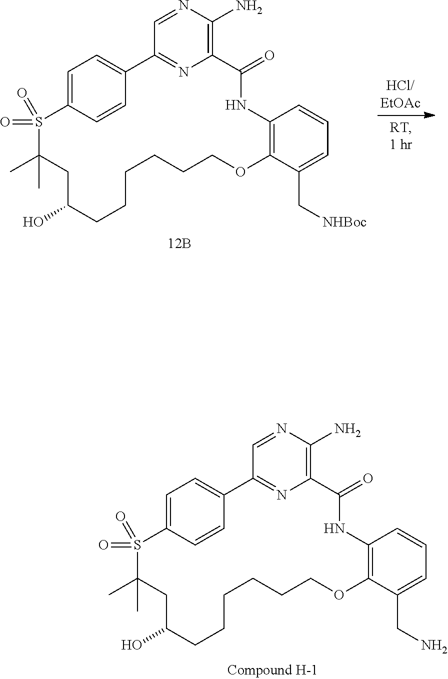 [SMILES: CC(C)(C)OC(=O)NCc1cccc2c1OCCCCCC[C@H](O)CC(C)(C)S(=O)(=O)c1ccc(cc1)-c1cnc(N)c(n1)C(=O)N2 ; CC1(C)C[C@@H](O)CCCCCCOc2c(CN)cccc2NC(=O)c2nc(cnc2N)-c2ccc(cc2)S1(=O)=O]

A mixture of tert-butylN-[[(22S)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (80 mg, 119.79 μmol, 1.0 eq.) in HCl/EtOAc (2 mL; 4 M) was stirred at 20° C. for 1 hour. The mixture was concentrated and the residue was purified by prep-HPLC (column: Phenomenex Luna 80*30 mm*3 μm; mobile phase: [water(HCl)-ACN]; B %: 20%-70%, 8 min) to give (22S)-5-amino-13-(aminomethyl)-22-hydroxy-24,24-dimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (42.58 mg, 70.48 μmol, 58.83% yield, 100% purity, HCl) as a yellow solid. ¹H NMR (400 MHz, DMSO-d₆) δ 10.40 (s, 1H), 9.13 (s, 1H), 8.46 (dd, J=2.3, 7.3 Hz, 1H), 8.41-8.28 (m, 5H), 8.12-7.90 (m, 4H), 7.36-7.28 (m, 2H), 4.12 (q, J=5.4 Hz, 2H), 3.96-3.82 (m, 2H), 3.55 (br d, J=9.4 Hz, 1H), 1.83-1.74 (m, 2H), 1.68 (br d, J=13.8 Hz, 1H), 1.51-1.38 (m, 7H), 1.38-1.31 (m, 4H), 1.20 (td, J=7.3, 14.5 Hz, 2H), 1.16-1.07 (m, 2H). ESI [M+H]=568.4.

### Example 9: Preparation of Compound G

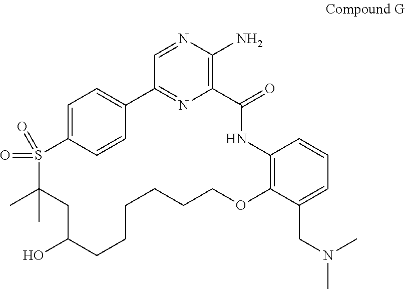 [SMILES: CN(C)Cc1cccc2c1OCCCCCCC(O)CC(C)(C)S(=O)(=O)c1ccc(cc1)-c1cnc(N)c(n1)C(=O)N2]

### (i) Preparation of Compound 2

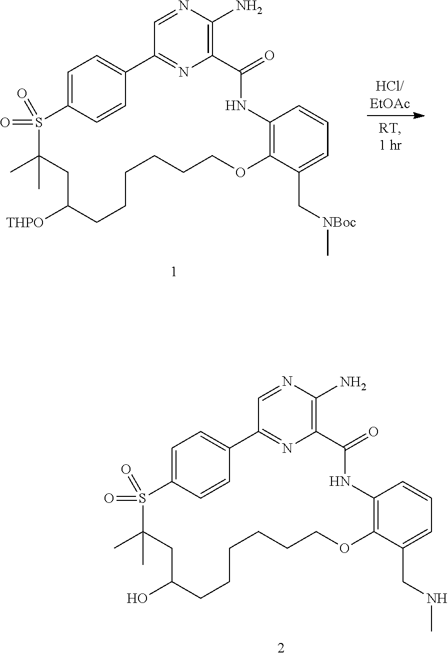

A mixture of tert-butyl N-[(5-amino-24,24-dimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (270 mg, 352.50 μmol, 1.0 eq.) in HCl/EtOAc (3 mL; 4 M) was stirred at 15° C. for 1 hour. The combined organic layers were concentrated to give 5-amino-22-hydroxy-24,24-dimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (210 mg, crude, HCl) as a yellow solid. ESI [M+H]=582.4.

### (ii) Preparation of Compound G

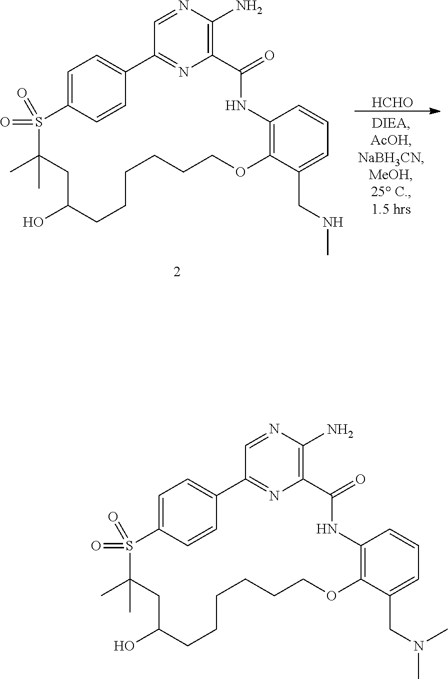 [SMILES: CNCc1cccc2c1OCCCCCCC(O)CC(C)(C)S(=O)(=O)c1ccc(cc1)-c1cnc(N)c(n1)C(=O)N2 ; "HCHO" ; CN(C)Cc1cccc2c1OCCCCCCC(O)CC(C)(C)S(=O)(=O)c1ccc(cc1)-c1cnc(N)c(n1)C(=O)N2]

To a solution of 5-amino-22-hydroxy-24,24-dimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (200 mg, 323.53 μmol, 1.0 eq., HCl) in MeOH (5 mL) was added DIEA (125.44 mg, 970.58 μmol, 3.0 eq.), AcOH (19.43 mg, 323.53 μmol, 1.0 eq.) and HCHO (52.51 mg, 647.05 μmol, 37% purity, 2.0 eq.). The mixture was stirred at 25° C. for 0.5 hour. Then NaBH₃CN (20.33 mg, 323.53 μmol, 1.0 eq.) was added. The mixture was stirred at 25° C. for 1 hour. The mixture was added sat. aq. Na₂CO₃ (5 mL) and extracted with dichloromethane (10 mL×2). The organic layer was dried over Na₂SO₄, filtered and concentrated to give a residue. Then the residue was purified by prep-HPLC (column: Phenomenex C18 80*40 mm*3 μm; mobile phase: [water(NH₄HCO₃)-ACN]; B %: 35%-65%, 8 min) to give 5-amino-13-[(dimethylamino)methyl]-22-hydroxy-24,24-dimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (42.42 mg, 70.58 μmol, 21.82% yield, 99.127% purity) as a yellow solid. ¹H NMR (400 MHz, DMSO-d₆) δ 10.39 (s, 1H), 9.10 (s, 1H), 8.41-8.30 (m, 3H), 8.22-7.83 (m, 4H), 7.17 (d, J=5.3 Hz, 2H), 4.50 (d, J=5.6 Hz, 1H), 3.96-3.80 (m, 2H), 3.55 (br d, J=4.0 Hz, 1H), 3.50-3.40 (m, 2H), 2.19 (s, 6H), 1.80-1.65 (m, 3H), 1.52-1.46 (m, 4H), 1.46-1.37 (m, 3H), 1.37-1.30 (m, 4H), 1.25-1.07 (m, 4H). ESI [M+H]=596.2.

### Example 10: Preparation of Compound G-2

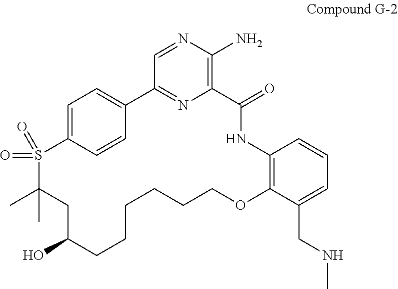 [SMILES: CNCc1cccc2c1OCCCCCC[C@@H](O)CC(C)(C)S(=O)(=O)c1ccc(cc1)-c1cnc(N)c(n1)C(=O)N2]

### (i) Preparation of Compound 2

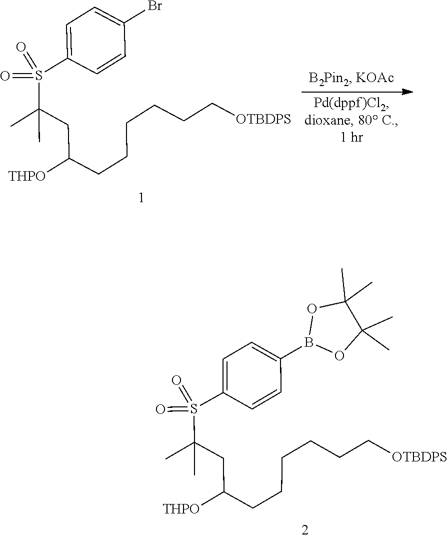

A mixture of [9-(4-bromophenyl)sulfonyl-9-methyl-7-tetrahydropyran-2-yloxy-decoxy]-tert-butyl-diphenyl-silane (2 g, 2.74 mmol, 1.0 eq.), 4,4,5,5-tetramethyl-2-(4,4,5,5-tetramethyl-1,3,2-dioxaborolan-2-yl)-1,3,2-dioxaborolane (765.42 mg, 3.01 mmol, 1.1 eq.), Pd(dppf)Cl₂ (200.50 mg, 274.02 μmol, 0.1 eq.) and KOAc (537.86 mg, 5.48 mmol, 2.0 eq.) in dioxane (30 mL) was stirred at 80° C. for 1 hour under N₂ atmosphere. The reaction solution was used directly to next step. ESI [M+Na⁺]=799.4.

(iii) Preparation of Compound 3

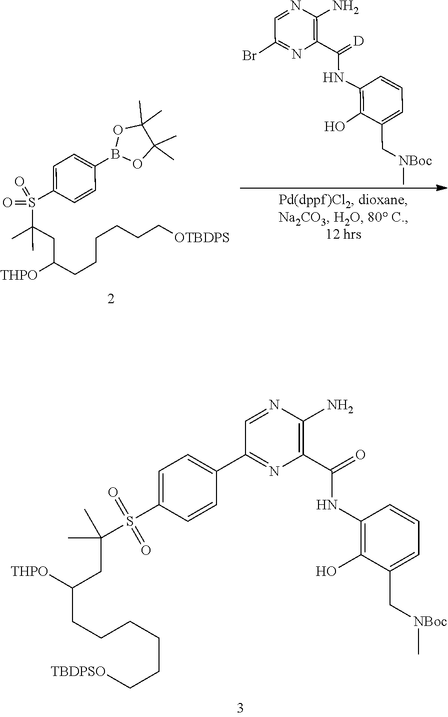

A mixture of tert-butyl N-[[3-[(3-amino-6-bromo-pyrazine-2-carbonyl)amino]-2-hydroxy-phenyl]methyl]-N-methyl-carbamate (1.10 g, 2.43 mmol, 0.9 eq.), tert-butyl-[9-methyl-7-tetrahydropyran-2-yloxy-9-[4-(4,4,5,5-tetramethyl-1,3,2-dioxaborolan-2-yl)phenyl]sulfonyl-decoxy]-diphenyl-silane (2.1 g, 2.70 mmol, 1.0 eq.), Na₂CO₃ (572.35 mg, 5.40 mmol, 2.0 eq.) and Pd(dppf)Cl₂ (395.12 mg, 540.00 μmol, 0.2 eq.) in dioxane (30 mL) and H₂O (6 mL) was stirred at 80° C. for 12 hours under N₂ atmosphere. The mixture was filtered, concentrated and the residue was diluted with H₂O (50 mL) and extracted with ethyl acetate (100 mL×2). Then the organic layers were dried over Na₂SO₄, filtered and concentrated to give a residue. The residue was purified by column chromatography (SiO₂, petroleum ether:ethyl acetate=100:1 to 0:1) to give tert-butyl N-[[3-[[3-amino-6-[4-[9-[tert-butyl(diphenyl)silyl]oxy-1,1-dimethyl-3-tetrahydropyran-2-yloxynonyl]sulfonylphenyl]pyrazine-2-carbonyl]amino]-2-hydroxy-phenyl]methyl]-N-methyl-carbamate (2.5 g, 2.45 mmol, 90.57% yield) as a yellow oil. ¹H NMR (400 MHz, chloroform-d) δ 10.73 (br s, 1H), 10.30-10.04 (m, 1H), 8.76 (br s, 1H), 8.45 (br d, J=3.7 Hz, 1H), 8.18 (br d, J=1.6 Hz, 2H), 7.99 (br d, J=2.6 Hz, 2H), 7.67 (br s, 4H), 7.40 (br s, 6H), 6.92 (br d, J=1.7 Hz, 2H), 4.67-4.46 (m, 1H), 4.37 (br s, 2H), 3.98-3.77 (m, 2H), 3.64 (br s, 2H), 3.54-3.34 (m, 1H), 2.92 (br s, 3H), 2.08-2.01 (m, 2H), 1.86 (br d, J=1.7 Hz, 4H), 1.81-1.63 (m, 6H), 1.51 (br s, 14H), 1.41-1.32 (m, 7H), 1.04 (br s, 9H). ESI [M+H−THP−Boc]=838.4.

(iii) Preparation of Compound 4

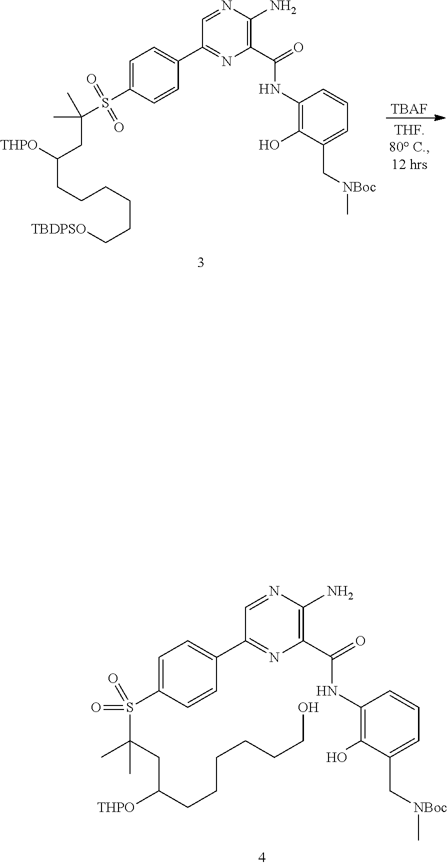

To a solution of tert-butyl N-[[3-[[3-amino-6-[4-[9-[tert-butyl(diphenyl)silyl]oxy-1,1-dimethyl-3-tetrahydropyran-2-yloxynonyl]sulfonylphenyl]pyrazine-2-carbonyl]amino]-2-hydroxy-phenyl]methyl]-N-methyl-carbamate (2.3 g, 2.25 mmol, 1.0 eq.) in THF (40 mL) was added TBAF (1 M, 4.50 mL, 2.0 eq.). The mixture was stirred at 40° C. for 12 hours. The mixture was concentrated and the residue was purified by column chromatography (SiO₂, petroleum ether:tetrahydrofuran=100:1 to 0:1) to give tert-butyl N-[[3-[[3-amino-6-[4-(9-hydroxy-1,1-dimethyl-3-tetrahydropyran-2-yloxy-nonyl)sulfonylphenyl]pyrazine-2-carbonyl]amino]-2-hydroxy-phenyl]methyl]-N-methyl-carbamate (1.4 g, 1.79 mmol, 79.38% yield) as a yellow solid. ESI [M+H−Boc]=684.3.

### (iv) Preparation of Compound 5

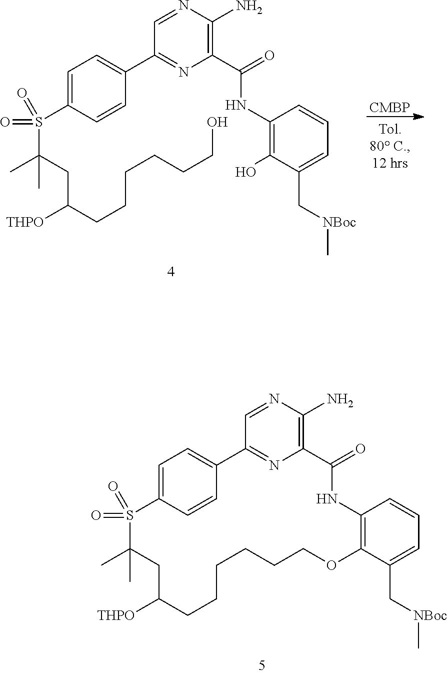

To a solution of tert-butyl N-[[3-[[3-amino-6-[4-(9-hydroxy-1,1-dimethyl-3-tetrahydropyran-2-yloxynonyl)sulfonylphenyl]pyrazine-2-carbonyl]amino]-2-hydroxy-phenyl]methyl]-N-methyl-carbamate (300 mg, 382.67 μmol, 1.0 eq.) in Tol. (5 mL) was added a solution of 2-(tributyl-λ⁵-phosphanylidene)acetonitrile (277.07 mg, 1.15 mmol, 3.0 eq.) in Tol. (1 mL) at 0° C. under N₂ atmosphere. The mixture was stirred at 80° C. for 12 hours under N₂ atmosphere. The combined organic layers were concentrated and purified by column chromatography (SiO₂, petroleum ether:tetrahydrofuran=100:1 to 0:1) to give tert-butyl N-[(5-amino-24,24-dimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (280 mg, crude) as a yellow gum. ESI [M+H−THP−Boc]=582.3.

### (v) Preparation of Compound 6

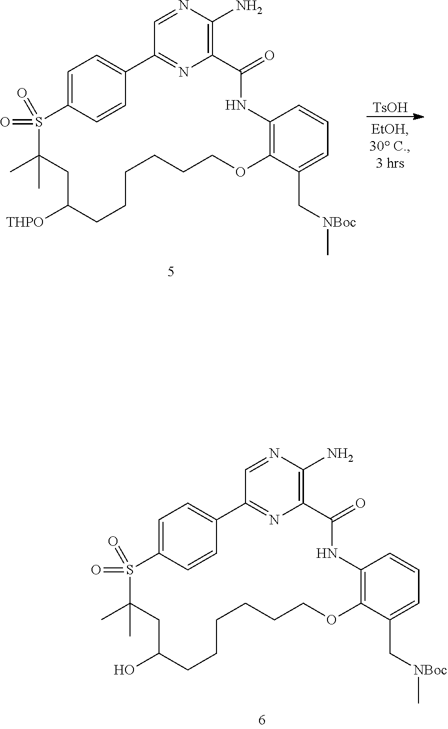

To a solution of tert-butyl N-[(5-amino-24,24-dimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (1.15 g, 1.50 mmol, 1.0 eq.) in EtOH (10 mL) was added 4-methylbenzenesulfonic acid; hydrate (285.59 mg, 1.50 mmol, 1.0 eq.). The mixture was stirred at 30° C. for 3 hours. The mixture was diluted with ice water, adjusted to pH=7 with sat. aq. Na₂CO₃ (10 mL) and extracted with ethyl acetate (50 mL×2). Then the reaction mixture was dried over Na₂SO₄, filtered and concentrated to give a residue. The residue was purified by column chromatography (SiO₂, petroleum ether:tetrahydrofuran=50:1 to 0:1) to give tert-butyl N-[(5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]-N-methyl-carbamate (630 mg, 923.97 μmol, 61.54% yield) as a yellow solid. ESI [M+H−Boc]=582.4.

### (vi) Preparation of Compounds 7A and 7B

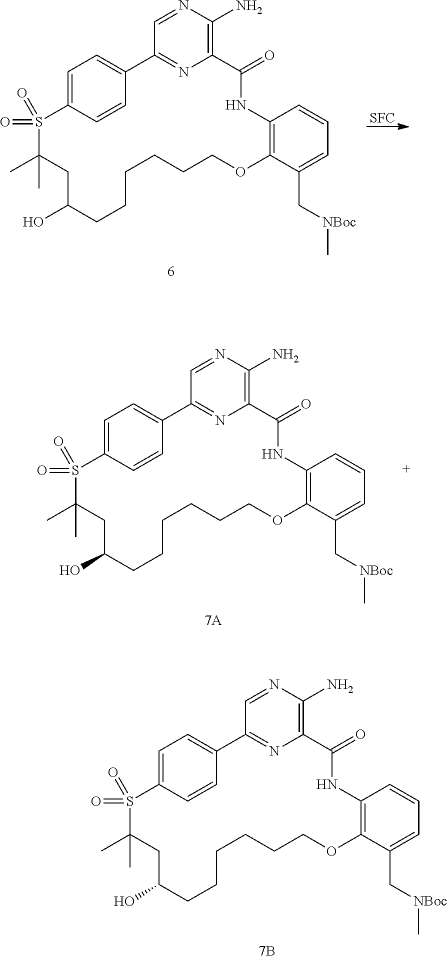

The racemic material was purified by SFC (column: Phenomenex-Cellulose-2 (250 mm*30 mm, 10 μm); mobile phase: [MeOH-ACN]; B %: 70%-70%, 15 min) to give arbitrarily assigned: tert-butyl N-[[(22R)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]-N-methyl-carbamate (Peak 1, retention time=2.255 min) (90 mg, 132.00 μmol, 45.00% yield, ee %=99.64%) as a yellow solid and tert-butyl N-[[(22S)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]-N-methyl-carbamate (Peak 2, retention time=3.724 min) (110 mg, 161.33 μmol, 55.00% yield, ee %=99%) as a yellow solid. ESI [M+H−Boc]=582.4.

(vii) Preparation of Compound G-2

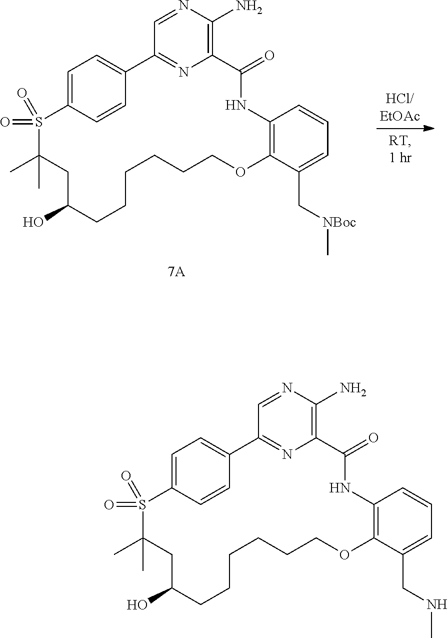 [SMILES: CN(Cc1cccc2c1OCCCCCC[C@@H](O)CC(C)(C)S(=O)(=O)c1ccc(cc1)-c1cnc(N)c(n1)C(=O)N2)C(=O)OC(C)(C)C ; CNCc1cccc2c1OCCCCCC[C@@H](O)CC(C)(C)S(=O)(=O)c1ccc(cc1)-c1cnc(N)c(n1)C(=O)N2]

To a solution of tert-butyl N-[[(22R)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]-N-methyl-carbamate (80 mg, 117.33 μmol, 1.0 eq.) in HCl/EtOAc (1 mL; 4 M). The mixture was stirred at 20° C. for 1 hour. The mixture was concentrated and purified by prep-HPLC (column: Phenomenex Luna 80*30 mm*3 μm; mobile phase: [water(HCl)-ACN]; B %: 20%-70%, 8 min) to give (22R)-5-amino-22-hydroxy-24,24-dimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (56.83 mg, 91.93 μmol, 78.35% yield, 100% purity, HCl) as a yellow solid. ¹H NMR (400 MHz, DMSO-d₆) δ 10.39 (s, 1H), 9.16-9.03 (m, 3H), 8.50-8.45 (m, 1H), 8.38 (d, J=8.4 Hz, 2H), 8.10-7.96 (m, 1H), 7.92 (d, J=8.4 Hz, 2H), 7.41-7.29 (m, 2H), 4.19 (br t, J=5.8 Hz, 2H), 3.95-3.81 (m, 2H), 3.61-3.50 (m, 1H), 2.63 (t, J=5.3 Hz, 3H), 1.85-1.72 (m, 2H), 1.67 (br d. J=13.8 Hz, 1H), 1.52-1.30 (m, 11H), 1.19 (td, J=6.8, 13.7 Hz, 2H), 1.15-1.06 (m, 2H). ESI [M+H]=582.4.

### Example 11: Preparation of Compound G-1

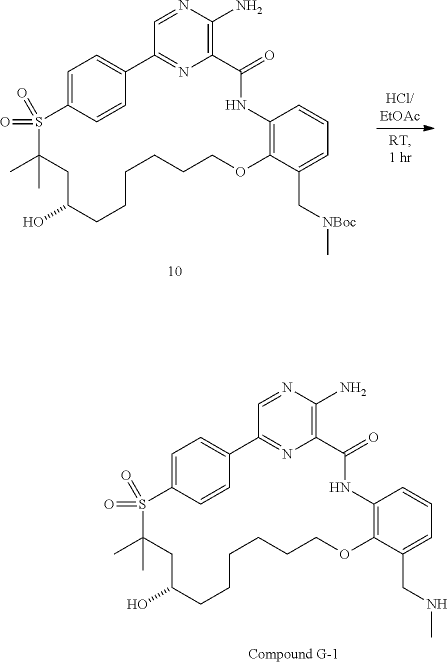 [SMILES: CN(Cc1cccc2c1OCCCCCC[C@H](O)CC(C)(C)S(=O)(=O)c1ccc(cc1)-c1cnc(N)c(n1)C(=O)N2)C(=O)OC(C)(C)C ; CNCc1cccc2c1OCCCCCC[C@H](O)CC(C)(C)S(=O)(=O)c1ccc(cc1)-c1cnc(N)c(n1)C(=O)N2]

To a solution of tert-butyl N-[[(22S)-5-amino-22-hydroxy-24,24-dimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]-N-methyl-carbamate (100 mg, 146.66 μmol, 1.0 eq.) in HCl/EtOAc (1 mL; 4 M). The mixture was stirred at 20° C. for 1 hour. The mixture was concentrated and purified by prep-HPLC (column: Phenomenex Luna 80*30 mm*3 μm; mobile phase: [water(HCl)-ACN]; B %: 20%-70%, 8 min) to give (22S)-5-amino-22-hydroxy-24,24-dimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (53.37 mg, 91.74 μmol, 62.56% yield, 100% purity) as a yellow solid. ¹H NMR (400 MHz, DMSO-d₆) δ 10.39 (s, 1H), 9.14 (s, 1H), 9.08 (br d, J=3.4 Hz, 2H), 8.48 (dd, J=1.4, 8.1 Hz, 1H), 8.38 (d, J=8.5 Hz, 2H), 8.10-7.96 (m, 1H), 7.92 (d, J=8.5 Hz, 2H), 7.48-7.25 (m, 2H), 4.19 (br t, J=5.8 Hz, 2H), 3.96-3.79 (m, 2H), 3.61-3.50 (m, 1H), 2.63 (t, J=5.3 Hz, 3H), 1.88-1.73 (m, 2H), 1.67 (br d, J=13.8 Hz, 1H), 1.51-1.30 (m, 11H), 1.20 (td, J=7.0, 13.9 Hz, 2H), 1.14-1.03 (m, 2H). ESI [M+H]=582.4.

### Example 12: Preparation of Compound C-2

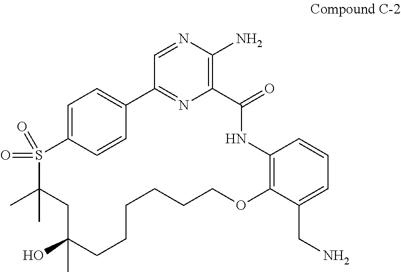 [SMILES: CC1(C)C[C@](C)(O)CCCCCCOc2c(CN)cccc2NC(=O)c2nc(cnc2N)-c2ccc(cc2)S1(=O)=O]

### (i) Preparation of Compound 2

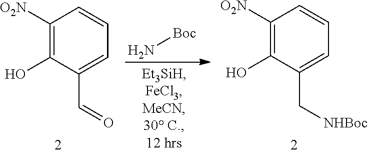

To a solution of 2-hydroxy-3-nitro-benzaldehyde (3 g, 17.95 mmol, 1.0 eq.), tert-butyl carbamate (6.31 g, 53.85 mmol, 3.0 eq.), FeCl₃ (5.82 g, 35.90 mmol, 2.08 mL, 2.0 eq.) in ACN (80 mL) was added Et₃SiH (14.61 g, 125.66 mmol, 20.07 mL, 7.0 eq.) under N₂ atmosphere. The mixture was stirred at 30° C. for 12 hours under N₂ atmosphere. The reaction mixture was quenched with 1N HCl (80 mL) and extracted with ethyl acetate (100 mL×2). The organic layer was washed with sat. aq. NaHCO₃ (100 mL×2), dried over Na₂SO₄, filtered and concentrated under reduced pressure to give a residue. The residue was purified by column chromatography (SiO₂, petroleum ether:ethyl acetate=100:1 to 0:1) to give tert-butyl N-[(2-hydroxy-3-nitro-phenyl)methyl]carbamate (2.7 g, 10.06 mmol, 56.07% yield) as a yellow solid. ESI [M+H−tBu]=213.1.

### (ii) Preparation of Compound 3

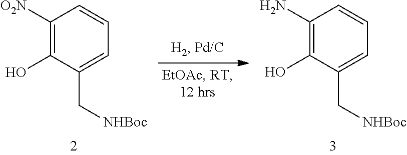 [SMILES: CC(C)(C)OC(=O)NCc1cccc([N+](=O)[O-])c1O ; CC(C)(C)OC(=O)NCc1cccc(N)c1O]

A mixture of tert-butyl N-[(2-hydroxy-3-nitro-phenyl)methyl]carbamate (2.4 g, 8.95 mmol, 1.0 eq.), Pd/C (10% purity) in EtOAc (160 mL) was degassed and purged with H₂ for 3 times, and then the mixture was stirred at 30° C. for 12 hours under H₂ (15 psi) atmosphere. The mixture was filtered, the filtrate was concentrated under reduced pressure to give tert-butyl N-[(3-amino-2-hydroxy-phenyl)methyl]carbamate (2.2 g, crude) as a yellow oil. ESI [M+H−tBu]=183.1.

(iii) Preparation of Compound 4

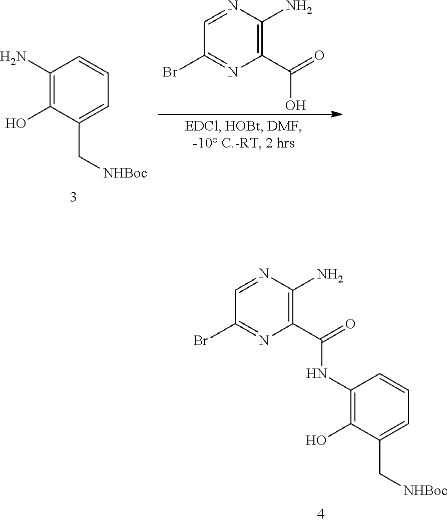 [SMILES: CC(C)(C)OC(=O)NCc1cccc(N)c1O ; Nc1ncc(Br)nc1C(=O)O ; CC(C)(C)OC(=O)NCc1cccc(NC(=O)c2nc(Br)cnc2N)c1O]

To a solution of 3-amino-6-bromo-pyrazine-2-carboxylic acid (1.8 g, 8.26 mmol, 1.0 eq.) and tert-butyl N-[(3-amino-2-hydroxyphenyl)methyl]carbamate (2.16 g, 9.08 mmol, 1.1 eq.) in DMF (40 mL) was added EDCI (1.90 g, 9.91 mmol, 1.2 eq.) and HOBt (557.81 mg, 4.13 mmol, 0.5 eq.) at −10° C. The mixture was stirred at 20° C. for 12 hours. The reaction mixture was diluted with H₂O (50 mL) and extracted with ethyl acetate (50 mL×2). The organic layer was washed with brine (20 mL×3), dried over Na₂SO₄, filtered and concentrated under reduced pressure to give a residue. The residue was purified by column chromatography (SiO₂, petroleum ether:tetrahydrofuran=100:1 to 0:1) and then triturated with EtOH (20 mL) to give tert-butyl N-[[3-[(3-amino-6-bromo-pyrazine-2-carbonyl)amino]-2-hydroxy-phenyl]methyl]carbamate (1.5 g, 3.42 mmol, 41.45% yield) as a yellow solid. ¹H NMR (400 MHz, DMSO-d₆) δ 10.18 (s, 1H), 9.62 (br s, 1H), 8.47 (s, 1H), 8.08 (br d, J=7.0 Hz, 1H), 7.82 (br s, 2H), 7.53 (br t, J=5.1 Hz, 1H), 6.91 (q, J=7.8 Hz, 2H), 4.16 (br d, J=6.1 Hz, 2H), 1.43 (s, 9H). ESI [M+H−tBu]=382.1/384.1.

### (iv) Preparation of Compound 6.

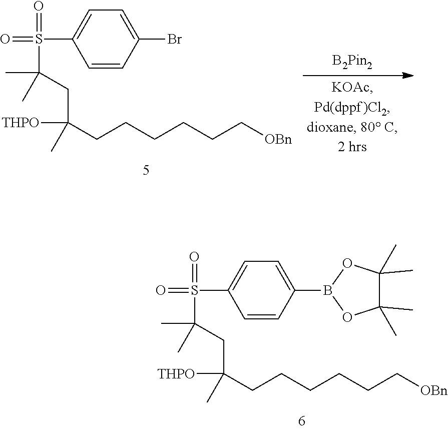

A mixture of 2-[7-benzyloxy-1-[2-(4-bromophenyl)sulfonyl-2-methyl-propyl]-1-methyl-heptoxy]tetrahydropyran (1.25 g, 2.10 mmol, 1.0 eq.), 4,4,5,5-tetramethyl-2-(4,4,5,5-tetramethyl-1,3,2-dioxaborolan-2-yl)-1,3,2-dioxaborolane (639.51 mg, 2.52 mmol, 1.2 eq.), KOAc (617.89 mg, 6.30 mmol, 3.0 eq.), Pd(dppf)Cl₂ (153.56 mg, 209.86 μmol, 0.1 eq.) in dioxane (20 mL) was degassed and purged with N₂ for 3 times, and then the mixture was stirred at 80° C. for 2 hours under N₂ atmosphere. The reaction solution was used directly to next step. ESI [M+Na]=665.3.

### (v) Preparation of Compound 7

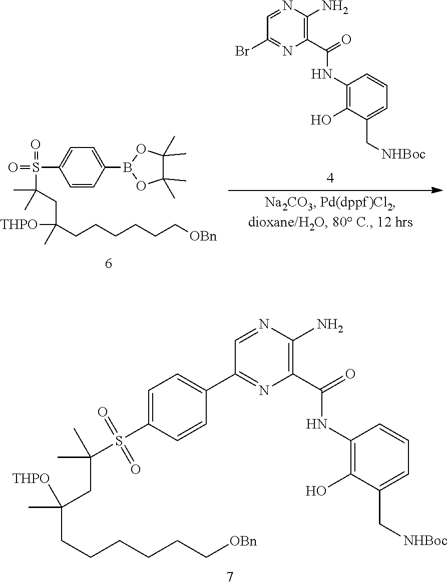

To a solution of 2-[4-(9-benzyloxy-1,1,3-trimethyl-3-tetrahydropyran-2-yloxy-nonyl)sulfonylphenyl]-4,4,5,5-tetramethyl-1,3,2-dioxaborolane (1.35 g, 2.10 mmol, 1.0 eq.) and tert-butyl N-[[3-[(3-amino-6-bromo-pyrazine-2-carbonyl)amino]-2-hydroxyphenyl]methyl]carbamate (920.61 mg, 2.10 mmol, 1.0 eq.) in dioxane (25 mL) and H₂O (5 mL) was added Pd(dppf)Cl₂ (153.70 mg, 210.05 μmol, 0.1 eq.) and Na₂CO₃ (333.95 mg, 3.15 mmol, 1.5 eq.). The mixture was stirred at 80° C. for 12 hours. The reaction mixture was concentrated to remove solvent and then added H₂O (50 mL), extracted with ethyl acetate (50 mL). The organic layer was washed with brine (50 mL), dried over Na₂SO₄, filtered and concentrated under reduced pressure to give a residue. The residue was purified by column chromatography (SiO₂, petroleum ether:ethyl acetate=50:1 to 1:1) to give tert-butyl N-[[3-[[3-amino-6-[4-(9-benzyloxy-1,1,3-trimethyl-3-tetrahydropyran-2-yloxynonyl)sulfonylphenyl]pyrazine-2-carbonyl]amino]-2-hydroxy-phenyl]methyl]carbamate (1.45 g, crude) as a yellow solid. ESI [M+H⁺]=874.3.

### (vi) Preparation of Compound 8

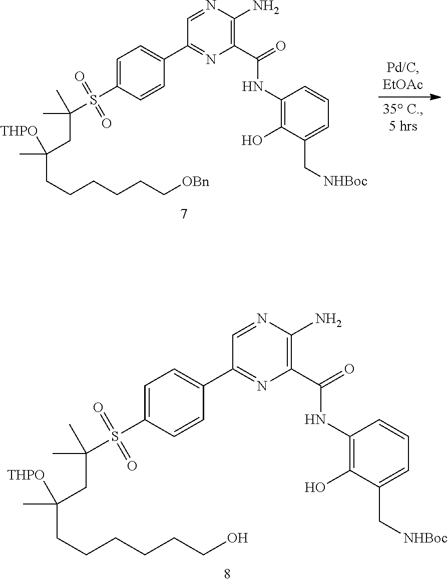

A mixture of tert-butyl N-[[3-[[3-amino-6-[4-(9-benzyloxy-1,1,3-trimethyl-3-tetrahydropyran-2-yloxynonyl)sulfonylphenyl]pyrazine-2-carbonyl]amino]-2-hydroxy-phenyl]methyl]carbamate (50 mg, 57.20 μmol, 1.0 eq.), Pd/C (50 mg, 10% purity) in EtOAc (2 mL) was degassed and purged with H₂ for 3 times, and then the mixture was stirred at 35° C. for 5 hours under H₂ (30 Psi) atmosphere. The reaction mixture was filtered, the filtrate was concentrated under reduced pressure to give a residue. The residue was purified by column chromatography (SiO₂, petroleum ether:tetrahydrofuran=50:1 to 1:1) to give tert-butyl N-[[3-[[3-amino-6-[4-(9-hydroxy-1,1,3-trimethyl-3-tetrahydropyran-2-yloxynonyl)sulfonylphenyl]pyrazine-2-carbonyl]amino]-2-hydroxy-phenyl]methyl]carbamate (45 mg, crude) as a yellow solid. ESI [M+Na⁺]=806.4.

(vii) Preparation of compound 9

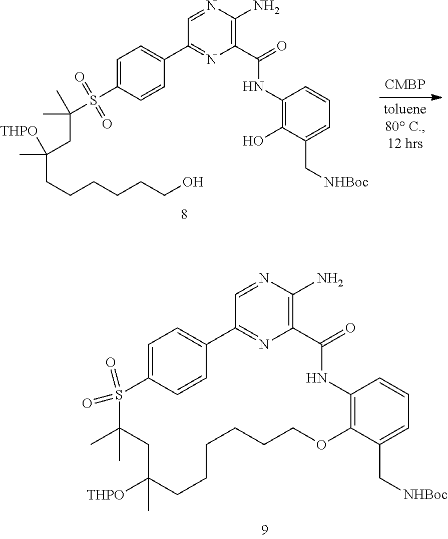

A mixture of tert-butyl N-[[3-[[3-amino-6-[4-(9-hydroxy-1,1,3-trimethyl-3-tetrahydropyran-2-yloxynonyl)sulfonylphenyl]pyrazine-2-carbonyl]amino]-2-hydroxy-phenyl]methyl]carbamate (70 mg, 89.29 μmol, 1.0 eq.), 2-(tributyl-λ⁵-phosphanylidene)acetonitrile (43.10 mg, 178.58 μmol, 2.0 eq.) in Tol. (2 mL) was degassed and purged with N₂ for 3 times, and then the mixture was stirred at 80° C. for 12 hours under N₂ atmosphere. The reaction mixture was diluted with H₂O (20 mL) and extracted with ethyl acetate (30 mL×2). The organic layer was dried over Na₂SO₄, filtered and concentrated under reduced pressure to give tert-butyl N-[(5-amino-22,24,24-trimethyl-7,25,25-trioxo-22-tetrahydropyran-2-yloxy-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (65 mg, crude) as a yellow solid. ESI [M+Na⁺]=788.4.

(viii) Preparation of Compound 11

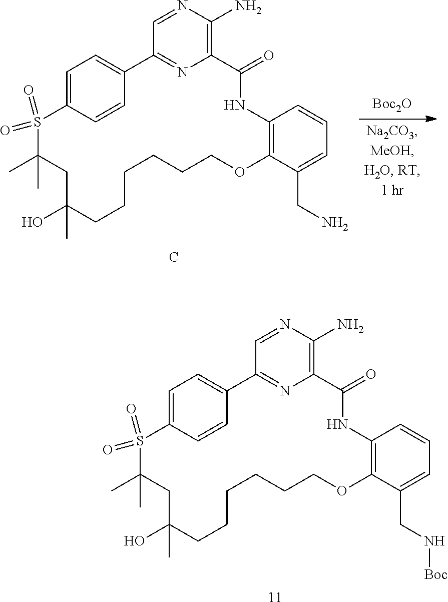

To a solution of 5-amino-13-(aminomethyl)-22-hydroxy-22,24,24-trimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (80 mg, 129.41 μmol, 1.0 eq., HCl) in MeOH (5 mL) and H₂O (1 mL) was added Na₂CO₃ (13.72 mg, 129.41 μmol, 1.0 eq.), Boc₂O (31.07 mg, 142.35 μmol, 32.70 μL, 1.1 eq.). The mixture was stirred at 20° C. for 1 hour. The mixture was added H₂O (20 mL) and extracted with ethyl acetate (40 mL×2). The organic layers were dried over Na₂SO₄, filtered and concentrated under reduced pressure to give a residue. The residue was purified by column chromatography (SiO₂, petroleum ether:ethyl acetate=100:1 to 1:2) to give tert-butyl N-[(5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (85 mg, 124.66 μmol, 96.33% yield) as a yellow solid. ESI [M+Na⁺]=704.3.

### (ix) Preparation of Compounds 12A and 12B

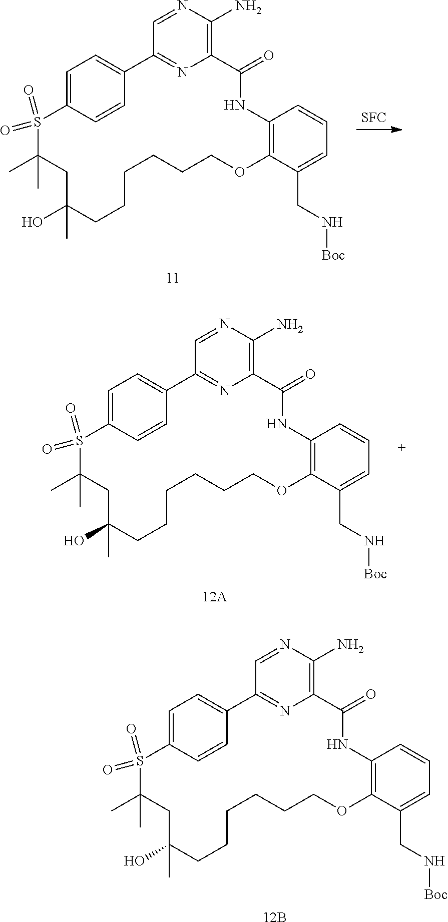

The racemic material was purified by SFC (column: DAICEL CHIRALPAK AD (250 mm*30 mm, 10 μm); mobile phase: [0.1% NH₃H₂O IPA]; B %: 50%-50%, 11 min) to give arbitrarily assigned: tert-butyl N-[[(22R)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (Peak 1, retention time=1.487 min) (45 mg, 66.00 μmol, 52.94% yield, ee %=100%) as a yellow solid and tert-butyl N-[[(22S)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (Peak 2, retention time=2.137 min) (40 mg, 58.66 μmol, 47.06% yield, ee %=98.44%) as a yellow solid. ESI [M+Na⁺]=704.3.

### (x) Preparation of Compound C-2

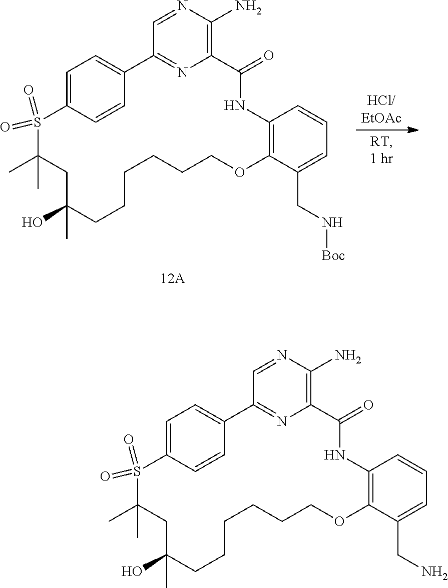 [SMILES: CC(C)(C)OC(=O)NCc1cccc2c1OCCCCCC[C@@](C)(O)CC(C)(C)S(=O)(=O)c1ccc(cc1)-c1cnc(N)c(n1)C(=O)N2 ; "HCl/EtOAc" ; "12A" ; CC1(C)C[C@](C)(O)CCCCCCOc2c(CN)cccc2NC(=O)c2nc(cnc2N)-c2ccc(cc2)S1(=O)=O]

A mixture of tert-butyl N-[[(22R)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (45 mg, 66.00 μmol, 1.0 eq.) in HCl/EtOAc (1 mL; 4 M) was stirred at 20° C. for 1 hour. The reaction mixture was concentrated under reduced pressure to give a residue. The residue was purified by prep-HPLC (column: Phenomenex Luna 80*30 mm*3 μm; mobile phase: [water(HCl)-ACN]; B %: 15%-40%, 8 min) to give (22R)-5-amino-13-(aminomethyl)-22-hydroxy-22,24,24-trimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (19 mg, 30.74 μmol, 46.57% yield, 100% purity, HCl) as a yellow solid. ¹H NMR (400 MHz, DMSO-d₆) δ 10.40 (s, 1H), 9.12 (s, 1H), 8.47-8.41 (m, 1H), 8.36 (d, J=8.5 Hz, 2H), 8.29 (br s, 3H), 8.10-7.88 (m, 4H), 7.35-7.28 (m, 2H), 4.36-4.19 (m, 1H), 4.16-4.08 (m, 2H), 3.95-3.82 (m, 2H), 1.86-1.73 (m, 3H), 1.53 (s, 3H), 1.48 (s, 3H), 1.43-1.31 (m, 4H), 1.28-1.10 (m, 6H), 1.07-0.95 (m, 2H). ESI [M+H]=582.3.

### Example 13: Preparation of Compound C-1

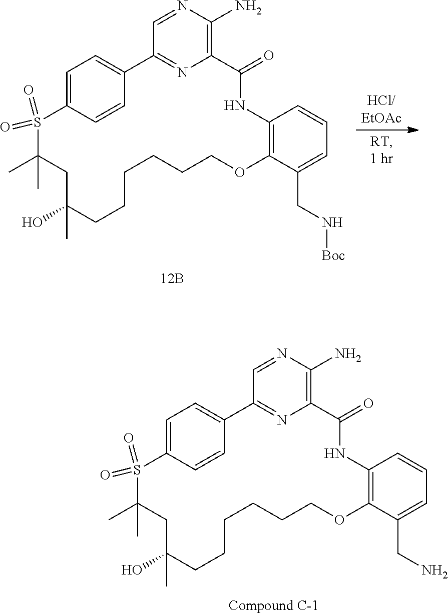 [SMILES: CC(C)(C)OC(=O)NCc1cccc2c1OCCCCCC[C@](C)(O)CC(C)(C)S(=O)(=O)c1ccc(cc1)-c1cnc(N)c(n1)C(=O)N2 ; "HCl/EtOAc" ; "12B" ; CC1(C)C[C@@](C)(O)CCCCCCOc2c(CN)cccc2NC(=O)c2nc(cnc2N)-c2ccc(cc2)S1(=O)=O ; "Compound C-1"]

A mixture of tert-butyl N-[[(22S)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl]methyl]carbamate (40 mg, 58.66 μmol, 1 eq.) in HCl/EtOAc (1 mL; 4 M) was stirred at 20° C. for 1 hour. The reaction mixture was concentrated under reduced pressure to give a residue. The residue was purified by prep-HPLC (column: Phenomenex Luna 80*30 mm*3 μm; mobile phase: [water(HCl)-ACN]; B %: 15%-40%, 8 min) to give (22S)-5-amino-13-(aminomethyl)-22-hydroxy-22,24,24-trimethyl-25,25-dioxo-15-oxa-25λ⁶-thia-4,8,31-triazatetracyclo[24.2.2.1^{2,6}.0^{9,14}]hentriaconta-1(28),2(31),3,5,9(14),10,12,26,29-nonaen-7-one (16.43 mg, 26.58 μmol, 45.30% yield, 100% purity, HCl) as a yellow solid. ¹H NMR (400 MHz, DMSO-d₆) δ 10.39 (s, 1H), 9.12 (s, 1H), 8.46-8.33 (m, 6H), 8.13-7.86 (m, 4H), 7.38-7.27 (m, 2H), 4.15-4.08 (m, 2H), 3.94-3.83 (m, 3H), 1.86-1.73 (m, 3H), 1.53 (s, 3H), 1.48 (s, 3H), 1.43-1.31 (m, 4H), 1.30-1.08 (m, 6H), 1.07-0.94 (m, 2H). ESI [M+H]=582.3.

### Example 14: Preparation of Compound A-2

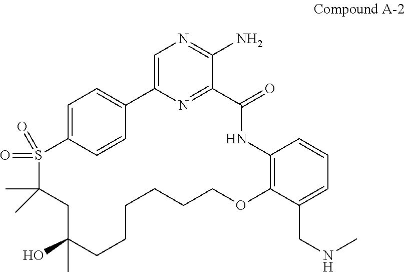 [SMILES: CNCc1cccc2c1OCCCCCC[C@@](C)(O)CC(C)(C)S(=O)(=O)c1ccc(cc1)-c1cnc(N)c(n1)C(=O)N2]

### (i) Preparation of Compound 20

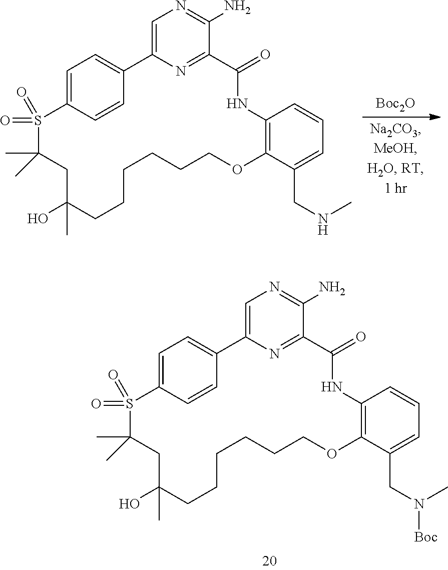 [SMILES: "Boc2O" ; CNCc1cccc2c1OCCCCCCC(C)(O)CC(C)(C)S(=O)(=O)c1ccc(cc1)-c1cnc(N)c(n1)C(=O)N2 ; CN(Cc1cccc2c1OCCCCCCC(C)(O)CC(C)(C)S(=O)(=O)c1ccc(cc1)-c1cnc(N)c(n1)C(=O)N2)C(=O)OC(C)(C)C]

To a solution of 5-amino-22-hydroxy-22,24,24-trimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-7-one (250 mg, 395.44 μmol, 1.0 eq., HCl) in MeOH (10 mL) and H₂O (2 ML) was added Na₂CO₃ (41.91 mg, 395.44 μmol, 1.0 eq.), Boc₂O (94.93 mg, 434.98 μmol, 99.93 μL, 1.1 eq.). The mixture was stirred at 20° C. for 1 hour. The mixture was added H₂O (40 mL) and extracted with Ethyl acetate (80 mL×2). The organic layers were dried over Na₂SO₄, filtered and concentrated under reduced pressure to give a residue. The residue was purified by column chromatography (SiO₂, petroleum ether:ethyl acetate=100:1 to 0:1) to give tert-butyl N-[(5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-13-yl)methyl]-N-methyl-carbamate (260 mg, 373.63 μmol, 94.49% yield) as a yellow solid. ESI [M+Na⁺]=718.4.

### (ii) Preparation of Compounds 21A and 21B.

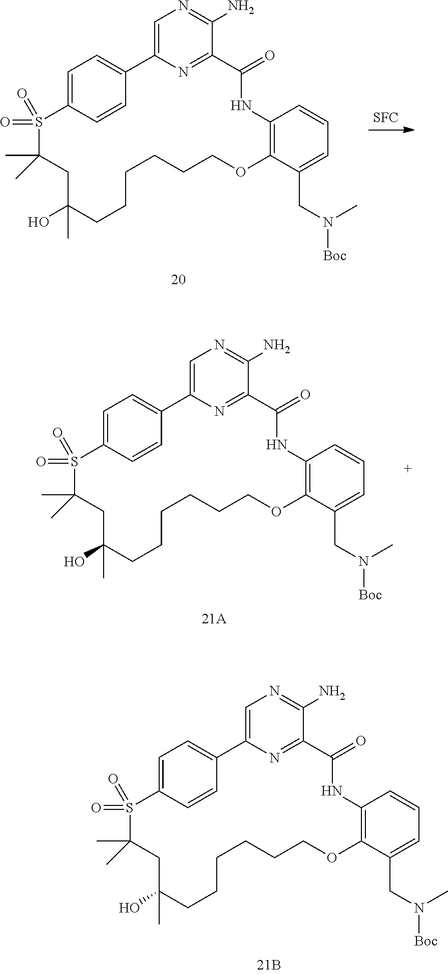

The racemic material was purified by SFC (column: Phenomenex-Cellulose-2 (250 mm*30 mm, 10 μm); mobile phase: [EtOH/ACN]; B %: 60%-60%, 20 min) to give arbitrarily assigned: tert-butyl N-[[(22R)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-13-yl]methyl]-N-methyl-carbamate (Peak 1, retention time=1.103 min) (130 mg, 186.82 μmol, 50.00% yield, ee %=100%) as a yellow solid and tert-butyl N-[[(22S)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-13-yl]methyl]-N-methyl-carbamate (Peak 2, retention time=2.602 min) (120 mg, 172.45 μmol, 46.15% yield, ee %=100%) as a yellow solid. ESI [M+Na⁺]=718.4.

(iii) Preparation of Compound A-2

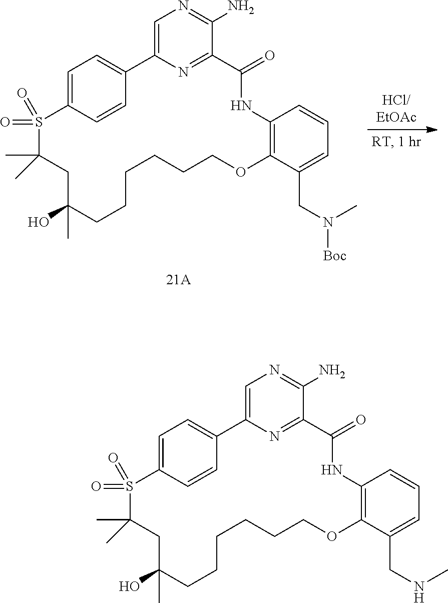 [SMILES: CN(Cc1cccc2c1OCCCCCC[C@@](C)(O)CC(C)(C)S(=O)(=O)c1ccc(cc1)-c1cnc(N)c(n1)C(=O)N2)C(=O)OC(C)(C)C ; "HCl/EtOAc" ; CNCc1cccc2c1OCCCCCC[C@@](C)(O)CC(C)(C)S(=O)(=O)c1ccc(cc1)-c1cnc(N)c(n1)C(=O)N2]

A mixture of tert-butyl N-[[(22R)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-13-yl]methyl]-N-methyl-carbamate (130 mg, 186.82 μmol, 1.0 eq.) in HCl/EtOAc (3 mL; 4 M) was stirred at 20° C. for 1 hour. The reaction mixture were concentrated under reduced pressure to give a residue. The residue was purified by prep-HPLC (column: Phenomenex Luna 80*30 mm*3 μm; mobile phase: [water(HCl)-ACN]; B %: 10%-40%, 8 min) to give (22R)-5-amino-22-hydroxy-22,24,24-trimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-7-one (79.75 mg, 126.14 μmol, 67.52% yield, 100% purity, HCl) as a yellow solid. ¹H NMR (400 MHz, DMSO-d₆) δ 10.39 (s, 1H), 9.12 (s, 3H), 8.46 (dd, J=1.4, 8.1 Hz, 1H), 8.37 (d, J=8.6 Hz, 2H), 8.15-7.83 (m, 4H), 7.42-7.38 (m, 1H), 7.36-7.29 (m, 1H), 4.19 (br t, J=5.7 Hz, 2H), 3.93-3.84 (m, 2H), 2.65-2.61 (m, 3H), 1.87-1.72 (m, 3H), 1.60 (br d, J=14.1 Hz, 1H), 1.54 (s, 3H), 1.48 (s, 3H), 1.41-1.28 (m, 4H), 1.26-1.09 (m, 5H), 1.07-0.90 (m, 2H). ESI [M+H]=596.2.

### Example 15: Preparation of Compound A-1

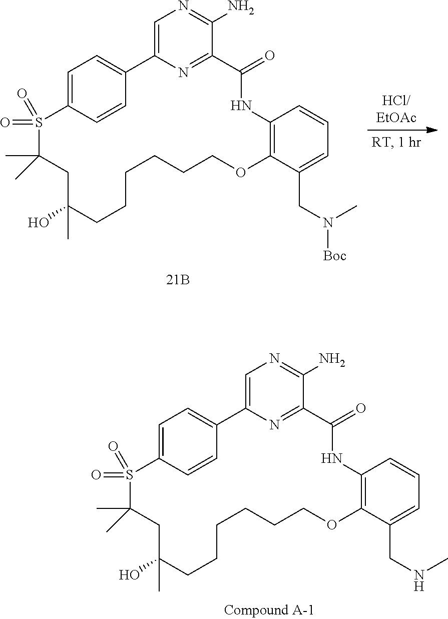 [SMILES: CN(Cc1cccc2c1OCCCCCC[C@](C)(O)CC(C)(C)S(=O)(=O)c1ccc(cc1)-c1cnc(N)c(n1)C(=O)N2)C(=O)OC(C)(C)C ; "HCl/EtOAc" ; CNCc1cccc2c1OCCCCCC[C@](C)(O)CC(C)(C)S(=O)(=O)c1ccc(cc1)-c1cnc(N)c(n1)C(=O)N2]

A mixture of tert-butyl N-[[(22S)-5-amino-22-hydroxy-22,24,24-trimethyl-7,25,25-trioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-13-yl]methyl]-N-methyl-carbamate (120 mg, 172.45 μmol, 1.0 eq.) in HCl/EtOAc (3 mL; 4 M) was stirred at 20° C. for 1 hour. The reaction mixture were concentrated under reduced pressure to give a residue. The residue was purified by prep-HPLC (column: Phenomenex Luna 80*30 mm*3 μm; mobile phase: [water(HCl)-ACN]; B %: 10%-40%, 8 min) to give (22S)-5-amino-22-hydroxy-22,24,24-trimethyl-13-(methylaminomethyl)-25,25-dioxo-15-oxa-25λ⁶-thia-4,8,31-triazatetracyclo[24.2.2.1^{2,6}.0^{9,14}]hentriaconta-1(28),2,4,6(31),9(14),10,12,26,29-nonaen-7-one (81.45 mg, 128.83 μmol, 74.71% yield, 100% purity, HCl) as a yellow solid. ¹H NMR (400 MHz, DMSO-d₆) δ 10.39 (s, 1H), 9.16-8.92 (m, 3H), 8.46 (dd, J=1.6, 7.9 Hz, 1H), 8.37 (d, J=8.5 Hz, 2H), 8.14-7.88 (m, 4H), 7.41-7.29 (m, 2H), 4.20 (br t, J=5.1 Hz, 2H), 3.94-3.82 (m, 2H), 2.64 (br t, J=5.2 Hz, 3H), 1.87-1.72 (m, 3H), 1.60 (br d, J=14.0 Hz, 1H), 1.54 (s, 3H), 1.48 (s, 3H), 1.41-1.30 (m, 4H), 1.27-1.10 (m, 5H), 1.07-0.90 (m, 2H). ESI [M+H]=596.2.

### Example 16: Preparation of Compound B-2

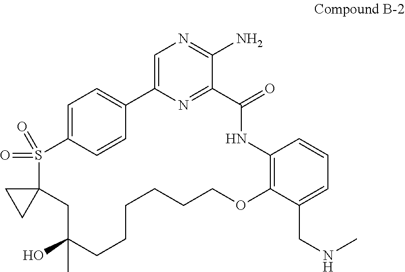 [SMILES: CNCc1cccc2c1OCCCCCC[C@@](C)(O)CC1(CC1)S(=O)(=O)c1ccc(cc1)-c1cnc(N)c(n1)C(=O)N2]

### (i) Preparation of Compound 2

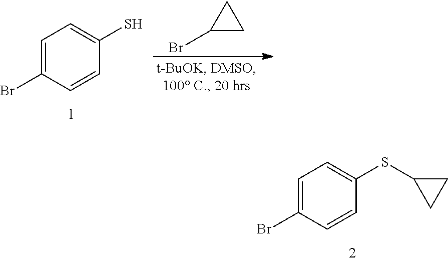

To a solution of 4-bromobenzenethiol (34 g, 179.83 mmol, 1.0 eq.) in DMSO (400 mL) was added t-BuOK (40.36 g, 359.65 mmol, 2.0 eq.) and bromocyclopropane (43.51 g, 359.65 mmol, 28.81 mL, 2.0 eq.). The mixture was stirred at 100° C. for 20 hours. The mixture was added ice water (1000 mL) and extracted with MTBE (1000 mL×3). The organic layers were dried over Na₂SO₄, filtered and concentrated to give 1-bromo-4-cyclopropylsulfanyl-benzene (32.5 g, crude) as a yellow oil. ¹H NMR (400 MHz, methanol-d₄) δ 7.31 (d, J=8.5 Hz, 2H), 7.17 (d, J=8.5 Hz, 2H), 2.17-2.04 (m, 1H), 1.04-0.95 (m, 2H), 0.55-0.47 (m, 2H).

### (ii) Preparation of Compound 3

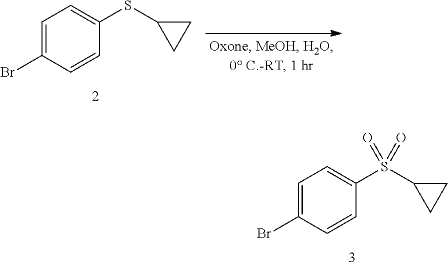 [SMILES: Brc1ccc(SC2CC2)cc1 ; O=S(=O)(c1ccc(Br)cc1)C1CC1]

To a solution of 1-bromo-4-cyclopropylsulfanyl-benzene (30 g, 130.93 mmol, 1.0 eq.) in MeOH (750 mL) was added a solution of oxone (185.12 g, 301.13 mmol, 2.3 eq.) in H₂O (750 mL) at 0° C. The mixture was stirred at 25° C. for 1 hour. The mixture was concentrated to remove MeOH, then extracted with ethyl acetate (1000 mL×2). The combined organic layers were dried over Na₂SO₄, filtered and concentrated to give the residue. The residue was triturated with MTBE (400 mL) to give 1-bromo-4-cyclopropylsulfonyl-benzene (28 g, 107.22 mmol, 81.90% yield) as a white solid. ¹H NMR (400 MHz, methanol-d₄) δ 7.85 (s, 4H), 2.73 (tt, J=4.8, 7.9 Hz, 1H), 1.30-1.24 (m, 2H), 1.15-1.08 (m, 2H). ESI [M+H]=261.0/263.0.

(iii) Preparation of Compound 5

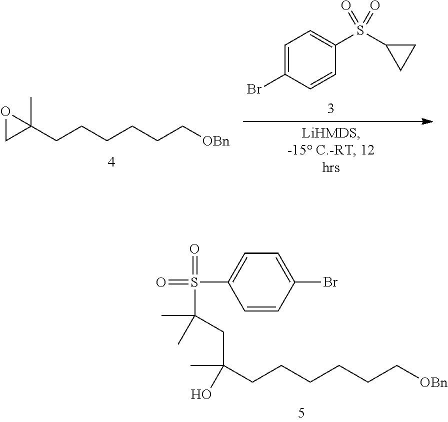 [SMILES: O=S(=O)(c1ccc(Br)cc1)C1CC1 ; CC1(CCCCCCOCc2ccccc2)CO1 ; CC(O)(CCCCCCOCc1ccccc1)CC(C)(C)S(=O)(=O)c1ccc(Br)cc1]

To a mixture of 2-(6-benzyloxyhexyl)-2-methyl-oxirane (11.41 g, 45.95 mmol, 1.2 eq.) and 1-bromo-4-cyclopropylsulfonyl-benzene (10 g, 38.29 mmol, 1.0 eq.) in THF (80 mL) was added dropwise LiHMDS (1 M, 57.44 mL, 1.5 eq.) at −20° C. under N₂ atmosphere. The mixture was stirred at 25° C. for 12 hours under N₂ atmosphere. The mixture was quenched with sat. aq. NH₄Cl (150 mL) at 0° C. and extracted with ethyl acetate (200 mL×2). The organic layers were dried over Na₂SO₄, filtered, concentrated and purified by column chromatography (SiO₂, petroleum ether:ethyl acetate=100:1 to 0:1) to give 8-benzyloxy-1-[1-(4-bromophenyl)sulfonylcyclopropyl]-2-methyl-octan-2-ol (9.42 g, 18.49 mmol, 48.28% yield) as a yellow oil. ¹H NMR (400 MHz, chloroform-d) δ 7.78-7.72 (m, 2H), 7.67-7.62 (m, 2H), 7.30-7.17 (m, 5H), 4.42 (s, 2H), 3.97 (s, 1H), 3.39 (t, J=6.6 Hz, 2H), 1.88-1.70 (m, 2H), 1.58-1.39 (m, 6H), 1.36-1.18 (m, 9H), 1.01-0.91 (m, 1H), 0.82 (ddd, J=5.4, 6.6, 8.9 Hz, 1H). ESI [M+Na⁺]=531.2/533.2.

### (iv) Preparation of Compound 6

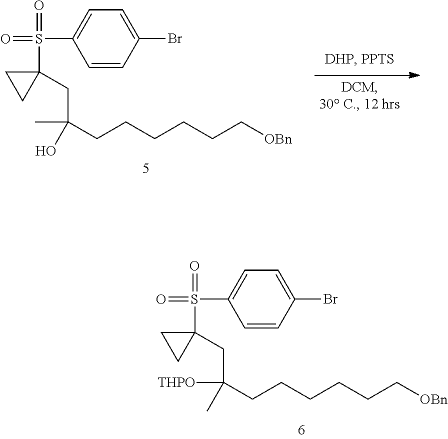

To a solution of 8-benzyloxy-1-[1-(4-bromophenyl)sulfonylcyclopropyl]-2-methyl-octan-2-ol (10 g, 19.63 mmol, 1.0 eq.) in DCM (100 mL) was added DHP (36.80 g, 437.47 mmol, 40.00 mL, 22.29 eq.) and PPTS (493.23 mg, 1.96 mmol, 0.1 eq.). The mixture was stirred at 30° C. for 12 hours under N₂ atmosphere. The mixture was added H₂O (50 mL) and extracted with dichloromethane (100 mL×2). The organic layers were dried over Na₂SO₄, filtered and concentrated to give a residue. Then the residue was purified by column chromatography (SiO₂, petroleum ether:ethyl acetate=100:1 to 0:1) to give 2-[7-benzyloxy-1-[[1-(4-bromophenyl)sulfonylcyclopropyl]methyl]-1-methyl-heptoxy]tetrahydropyran (13 g, crude) as a yellow oil. ESI [M+Na⁺]=615.3/617.3.

### (v) Preparation of Compound 7

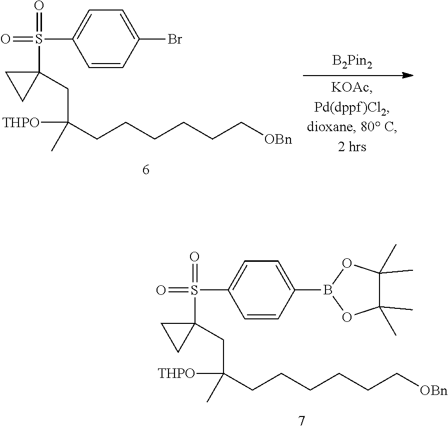

A mixture of 2-[7-benzyloxy-1-[[1-(4-bromophenyl)sulfonylcyclopropyl]methyl]-1-methyl-heptoxy]tetrahydropyran (13 g, 21.90 mmol, 1.0 eq.), 4,4,5,5-tetramethyl-2-(4,4,5,5-tetramethyl-1,3,2-dioxaborolan-2-yl)-1,3,2-dioxaborolane (6.12 g, 24.09 mmol, 1.1 eq.), Pd(dppf)Cl₂ (1.60 g, 2.19 mmol, 0.1 eq.), KOAc (4.30 g, 43.80 mmol, 2.0 eq.) in dioxane (230 mL) was stirred at 80° C. for 2 hours under N₂ atmosphere. The reaction solution was used directly to next step. ESI [M+Na⁺]=663.4.

### (vi) Preparation of Compound 8

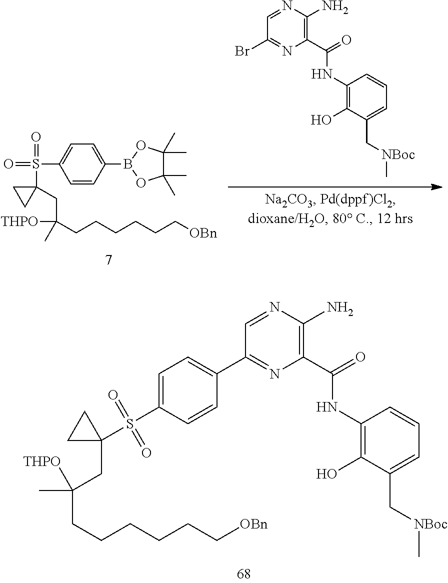

A mixture of tert-butyl N-[[3-[(3-amino-6-bromo-pyrazine-2-carbonyl)amino]-2-hydroxy-phenyl]methyl]-N-methyl-carbamate (9.88 g, 21.85 mmol, 1.0 eq.), 2-[4-[1-(8-benzyloxy-2-methyl-2-tetrahydropyran-2-yloxy-octyl)cyclopropyl]sulfonylphenyl]-4,4,5,5-tetramethyl-1,3,2-dioxaborolane (14 g, 21.85 mmol, 1.0 eq.), Na₂CO₃ (3.47 g, 32.78 mmol, 1.5 eq.), Pd(dppf)Cl₂ (1.60 g, 2.19 mmol, 0.1 eq.) in dioxane (230 mL) and H₂O (45 mL) was stirred at 80° C. for 12 hours under N₂ atmosphere. The reaction mixture was concentrated and then added water (200 mL), extracted with ethyl acetate (200 mL×2). The combined organic layers were dried over Na₂SO₄, filtered and concentrated to give a residue. Then the residue was purified by column chromatography (SiO₂, petroleum ether:ethyl acetate=100:1 to 1:1) to give tert-butyl N-[[3-[[3-amino-6-[4-[1-(8-benzyloxy-2-methyl-2-tetrahydropyran-2-yloxyoctyl)cyclopropyl]sulfonylphenyl]pyrazine-2-carbonyl]amino]-2-hydroxy-phenyl]methyl]-N-methyl-carbamate (18.2 g, 20.54 mmol, 93.99% yield) as a yellow oil. ESI [M+H]=886.5.

(vii) Preparation of Compound 9

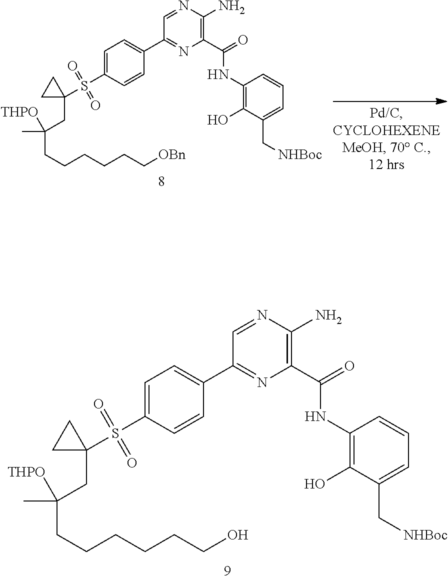

A mixture of tert-butyl N-[[3-[[3-amino-6-[4-[1-(8-benzyloxy-2-methyl-2-tetrahydropyran-2-yloxyoctyl)cyclopropyl]sulfonylphenyl]pyrazine-2-carbonyl]amino]-2-hydroxy-phenyl]methyl]-N-methyl-carbamate (6.5 g, 7.34 mmol, 1.0 eq.), Pd/C (6 g, 10% purity) in MeOH (300 mL) and cyclohexene (60 mL) was stirred at 70° C. for 12 hours. The mixture was filtered and the filtrate was concentrated in vacuo. The residue was purified by column chromatography (SiO₂, petroleum ether:tetrahydrofuran=100:1 to 0:1) to give tert-butyl N-[[3-[[3-amino-6-[4-[1-(8-hydroxy-2-methyl-2-tetrahydropyran-2-yloxyoctyl)cyclopropyl]sulfonylphenyl]pyrazine-2-carbonyl]amino]-2-hydroxy-phenyl]methyl]-N-methyl-carbamate (6.2 g, crude) as a yellow solid. ESI [M+H−Boc]=696.4.

(viii) Preparation of Compound 10

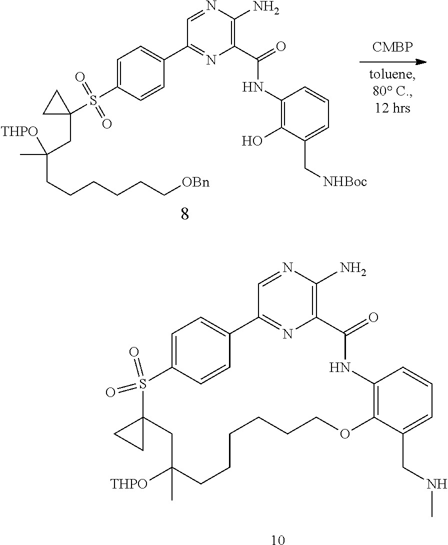

To a solution of tert-butyl N-[[3-[[3-amino-6-[4-[1-(8-hydroxy-2-methyl-2-tetrahydropyran-2-yloxyoctyl)cyclopropyl]sulfonylphenyl]pyrazine-2-carbonyl]amino]-2-hydroxy-phenyl]methyl]-N-methyl-carbamate (6.2 g, 7.79 mmol, 1.0 eq.) in Tol. (110 mL) was added a solution of 2-(tributyl-V-phosphanylidene)acetonitrile (5.64 g, 23.37 mmol, 3.0 eq.) in Tol. (10 mL) at 0° C. The mixture was stirred at 80° C. for 12 hours. The reaction mixture was concentrated in vacuo. The residue was purified by column chromatography (SiO₂, petroleum ether:ethyl acetate=100:1 to 0:1) to give tert-butyl N-[(5-amino-22-methyl-7,25,25-trioxo-22-tetrahydropyran-2-yloxy-spiro[15-oxa-25λ⁶thia4,8,31triazatetracyclo[24.2.2.1^{2,6}.0^{9,14}]hentriacontal(28),2(31),3,5,9(14),10,12,26,29-nonaene-24,1′-cyclopropane]-13-yl)methyl]-Nmethyl-carbamate (5.2 g, 6.68 mmol, 85.81% yield) as a yellow solid. ESI [M+H−Boc]=678.4.

### (ix) Preparation of Compound 13

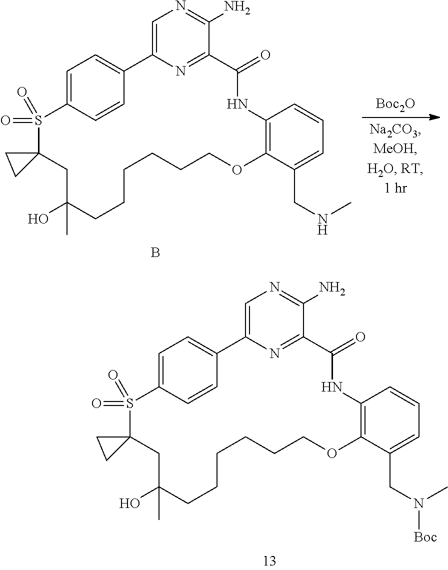

To a solution of 5-amino-22-hydroxy-22-methyl-13-(methylaminomethyl)-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (180 mg, 285.63 μmol, 1.0 eq., HCl) in MeOH (10 mL) and H₂O (2 mL) was added Na₂CO₃ (30.27 mg, 285.63 μmol, 1.0 eq.) and Boc₂O (68.57 mg, 314.19 μmol, 72.18 μL, 1.1 eq.). The mixture was stirred at 20° C. for 1 hour. The mixture was added H₂O (20 mL) and extracted with ethyl acetate (50 mL×2). The organic layer was dried over Na₂SO₄, filtered and concentrated to give a residue. Then the residue was purified by column chromatography (SiO₂, petroleum ether:ethyl acetate=100:1 to 0:1) to give tert-butyl N-[(5-amino-22-hydroxy-22-methyl-7,25,25-trioxo-spiro[15-oxa-25λ⁶-thia-4,8,31-triazatetracyclo[24.2.2.1^{2,6}.0^{9,14}]hentriaconta-1(28),2(31),3,5,9(14),10,12,26,29-nonaene-24,1′-cyclopropane]-13-yl)methyl]-Nmethyl-carbamate (160 mg, 230.60 μmol, 80.73% yield) as a yellow solid. ESI [M+H−Boc]=594.4.

### (x) Preparation of Compounds 14A and 14B

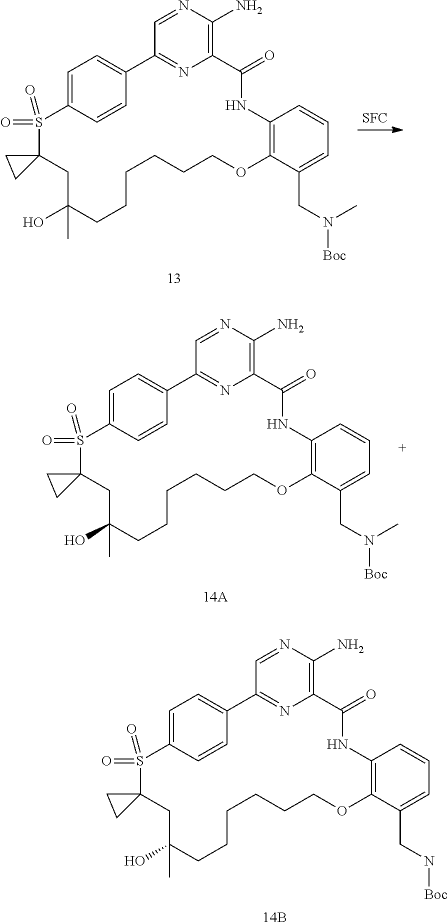

The racemic material was purified by SFC (column: DAICEL CHIRALPAKAD (250 mm*30 mm, 10 μm); mobile phase: [0.1% NH₃H₂O EtOH]: B %: 54%-54%, 8 min) to give arbitrarily assigned: tert-butyl N-[[(22R)-5-amino-22-hydroxy-22-methyl-7,25,25-trioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-13-yl]methyl]-N-methyl-carbamate (Peak 1, retention time=1.484 min) (70 mg, 100.89 μmol, 43.75% yield, ee %=100%) as a yellow solid and tert-butyl N-[[(22S)-5-amino-22-hydroxy-22-methyl-7,25,25-trioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-13-yl]methyl]-N-methyl-carbamate (Peak 2, retention time=1.888 min) (90 mg, 129.71 μmol, 56.25% yield, ee %=99.66%) as a yellow solid. [M+H−Boc]=594.3.

### (xi) Preparation of Compound B-2

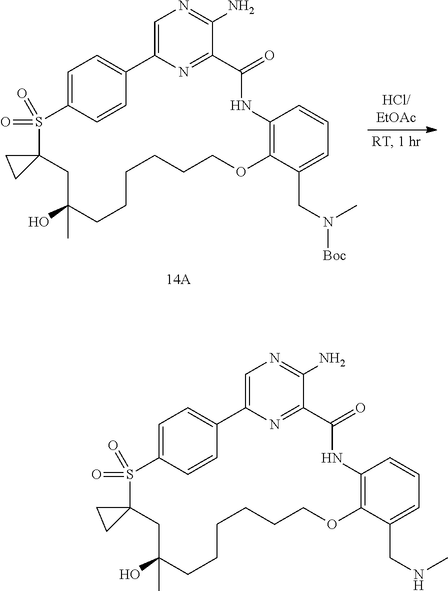 [SMILES: CN(Cc1cccc2c1OCCCCCC[C@@](C)(O)CC1(CC1)S(=O)(=O)c1ccc(cc1)-c1cnc(N)c(n1)C(=O)N2)C(=O)OC(C)(C)C ; "HCl/EtOAc" ; CNCc1cccc2c1OCCCCCC[C@@](C)(O)CC1(CC1)S(=O)(=O)c1ccc(cc1)-c1cnc(N)c(n1)C(=O)N2]

A mixture of tert-butyl N-[[(22R)-5-amino-22-hydroxy-22-methyl-7,25,25-trioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-13-yl]methyl]-Nmethyl-carbamate (65 mg, 93.68 μmol, 1.0 eq.) in HCl/EtOAc (2 mL; 4 M) was stirred at 20° C. for 1 hour. The reaction mixture was concentrated and the residue was purified by prep-HPLC (column: Phenomenex Luna 80*30 mm*3 μm; mobile phase: [water(HCl)-ACN]; B %: 10%-50%, 8 min) to give (22R)-5-amino-22-hydroxy-22-methyl-13-(methylaminomethyl)-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (31.72 mg, 50.33 μmol, 53.73% yield, 100% purity, HCl) as a yellow solid. ¹H NMR (400 MHz, DMSO-d₆) δ 10.33 (s, 1H), 9.08 (s, 3H), 8.37-8.28 (m, 3H), 7.98 (br d, J=8.3 Hz, 3H), 7.42 (d, J=7.8 Hz, 1H), 7.35-7.29 (m, 1H), 4.19 (br t, J=5.1 Hz, 3H), 3.91-3.76 (m, 2H), 2.64 (br t, J=4.9 Hz, 3H), 2.09-2.01 (m, 1H), 1.97-1.90 (m, 1H), 1.81-1.69 (m, 1H), 1.68-1.54 (m, 3H), 1.53-1.43 (m, 2H), 1.36 (dt, J=6.3, 11.7 Hz, 1H), 1.28-1.17 (m, 1H), 1.15-1.00 (m, 2H), 0.99-0.67 (m, 7H). ESI [M+H]=594.3.

### Example 17: Preparation of Compound B-1

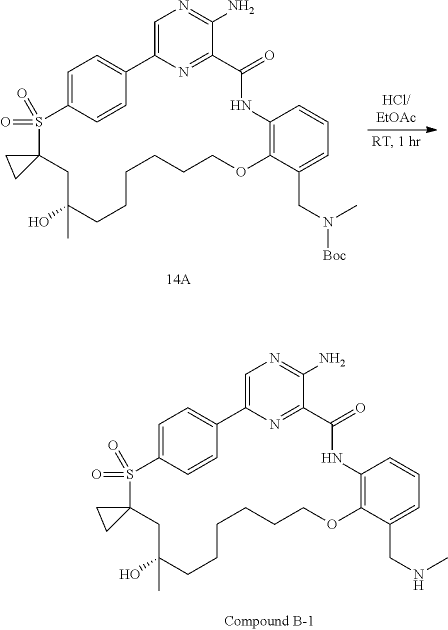 [SMILES: CN(Cc1cccc2c1OCCCCCC[C@](C)(O)CC1(CC1)S(=O)(=O)c1ccc(cc1)-c1cnc(N)c(n1)C(=O)N2)C(=O)OC(C)(C)C ; "HCl/EtOAc" ; CNCc1cccc2c1OCCCCCC[C@](C)(O)CC1(CC1)S(=O)(=O)c1ccc(cc1)-c1cnc(N)c(n1)C(=O)N2]

A mixture of tert-butyl N-[[(22S)-5-amino-22-hydroxy-22-methyl-7,25,25-trioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-13-yl]methyl]-Nmethyl-carbamate (85 mg, 122.50 μmol, 1.0 eq.) in HCl/EtOAc (2 mL; 4 M) was stirred at 20° C. for 1 hour. The reaction mixture was concentrated and the residue was purified by prep-HPLC (column: Phenomenex Luna 80*30 mm*3 μm; mobile phase: [water(HCl)-ACN]; B %: 10%-50%, 8 min) to give (22S)-5-amino-22-hydroxy-22-methyl-13-(methylaminomethyl)-25,25-dioxo-spiro[15-oxa-25λ⁶-thia-4,8,31-triazatetracyclo[24.2.2.1^{2,6}.0^{9,14}]hentriaconta-1(28),2,4,6(31),9(14),10,12,26,29-nonaene-24,1′-cyclopropane]-7-one (37.42 mg, 59.38 μmol, 48.47% yield, 100% purity, HCl) as a yellow solid. ¹H NMR (400 MHz, DMSO-d₆) δ 10.33 (s, 1H), 9.09 (s, 1H), 8.96 (br d, J=5.8 Hz, 2H), 8.40-8.27 (m, 3H), 7.99 (br d, J=8.4 Hz, 3H), 7.42-7.29 (m, 2H), 4.28-4.15 (m, 3H), 3.90-3.76 (m, 2H), 2.65 (br t, J=5.1 Hz, 3H), 2.08-2.01 (m, 1H), 1.98-1.89 (m, 1H), 1.83-1.70 (m, 1H), 1.67-1.55 (m, 3H), 1.53-1.42 (m, 2H), 1.41-1.30 (m, 1H), 1.27-1.17 (m, 1H), 1.16-1.02 (m, 2H), 0.89 (s, 7H). ESI [M+H]=594.3.

### Example 18: Preparation of Compound J-1

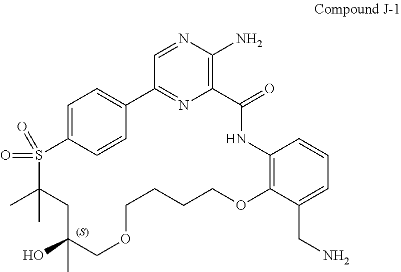

(i) Preparation of compound 2

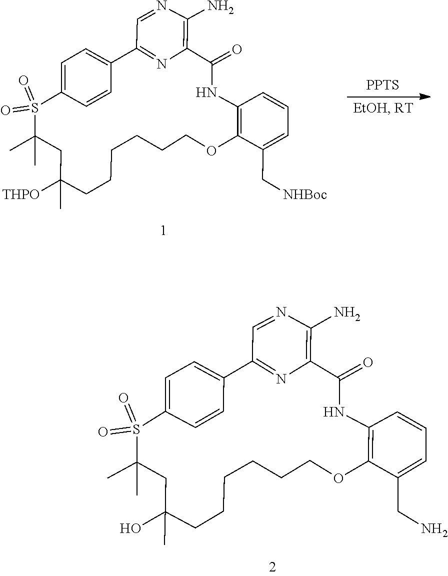

To a solution of tert-butyl N-[(32-amino-36,36,37-trimethyl-34,53,53-trioxo-37-tetrahydropyran-2-yloxy-48,49-dioxa-53thia-40,41,42-triazatetracyclohentriaconta-6(8),7(27),9(25),10(12),11(26),13(40),28(30),29(41),31-nonaen-27-yl)methyl]carbamate (120 mg, 156.26 μmol, 1.0 eq.) in EtOH (5 mL) was added TsOH·H₂O (5.94 mg, 31.25 μmol, 0.2 eq.). The mixture was stirred at 30° C. for 1 hour. The reaction mixture was concentrated to give tert-butyl N-[(5-amino-22-hydroxy-22,24,24-trimethyl-7,25,25-trioxo-15,20-dioxa-25λ⁶-thia-4,8,31-triazatetracyclo[24.2.2.1^{2,6}.0^{9,14}]hentriaconta-1(28),2(31),3,5,9(14),10,12,26,29-nonaen-13-yl)methyl]carbamate (110 mg, 160.86 μmol, 102.94% yield) as a yellow solid. ESI [M+H]=684.3.

### (ii) Preparation of Compounds 3A and 3B

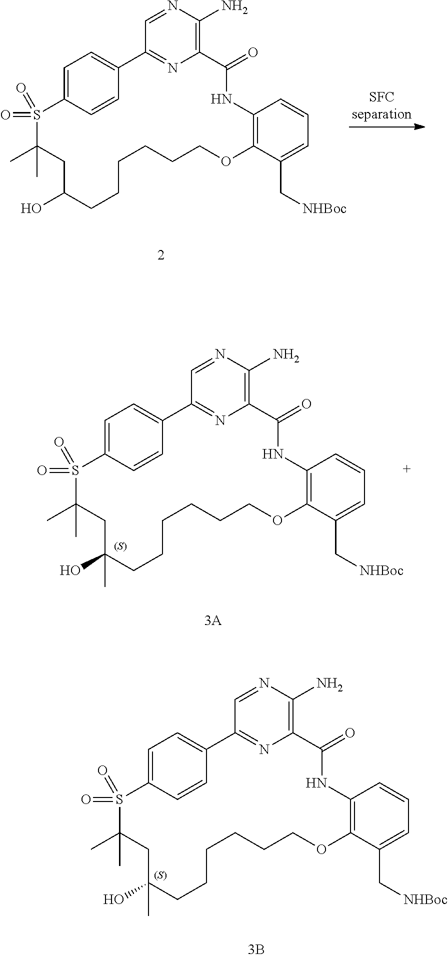

The racemic material was purified by SFC (column: DAICEL CHIRALPAK IC (250 mm*50 mm, 10 μm); mobile phase: [0.1% NH₃H₂O MEOH]; B %: 50%-50%, 40 min) to give arbitrarily assigned: tert-butyl N-[[(32S)-28-amino-32-hydroxy-31,31,32-trimethyl-29,47,47-trioxo-44,45-dioxa-47thia-35,36,37-triazatetracyclohentriaconta-6(8),7(23),9(21),10(12),11(22),13(35),24(26),25(36),27-nonaen-23-yl]methyl]carbamate (Peak 1, retention time=3.89 min) (50 mg, 73.12 μmol, 93.58% yield, ee %=100%) as a yellow solid and tert-butyl N-[[(32R)-28-amino-32-hydroxy-31,31,32-trimethyl-29,47,47-trioxo-44,45-dioxa-47thia-35,36,37-triazatetracyclohentriaconta-6(8),7(23),9(21),10(12),11(22),13(35),24(26),25(36),27-nonaen-23-yl]methyl]carbamate (Peak 2, retention time=6.16 min) (50 mg, 73.12 μmol, 93.58% yield, ee %=100%) as a yellow solid. ESI [M+H]=684.3.

(iii) Preparation of Compound J-1

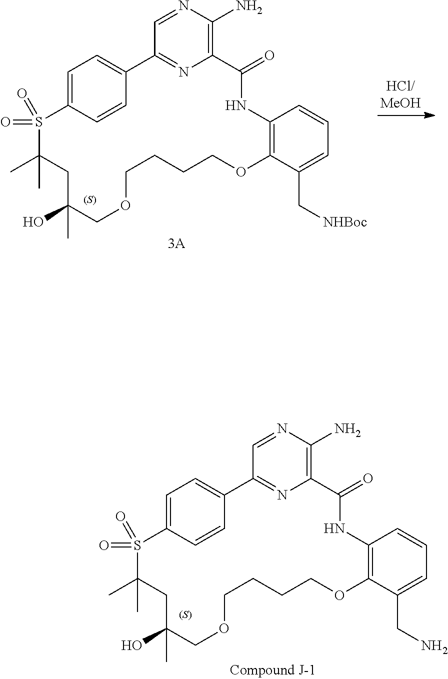

A solution of tert-butyl N-[[(32S)-28-amino-32-hydroxy-31,31,32-trimethyl-29,47,47-trioxo-44,45-dioxa-47thia-35,36,37-triazatetracyclohentriaconta-6(8),7(23),9(21),10(12),11(22),13(35),24(26),25(36),27-nonaen-23-yl]methyl]carbamate (50 mg, 73.12 μmol, 1.0 eq.) in HCl/MeOH (1 mL; 4M) was stirred at 30° C. for 0.5 hour. The reaction mixture was concentrated and the residue was purified by prep-HPLC (column: Phenomenex Luna C18 100*30 mm*5 μm; mobile phase: [water (0.04% HCl)-ACN]; B %: 15%-45%, 10 min) to give (28S)-25-amino-20-(aminomethyl)-28-hydroxy-27,27,28-trimethyl-40,40-dioxo-38,39-dioxa-40thia-31,32,33-triazatetracyclohentriaconta-3(5),4(20),6(18),7(9),8(19),10(31),21(23),22(32),24-nonaen-26-one (29.3 mg, 47.13 μmol, 64.46% yield, 99.76% purity, HCl) as a yellow solid. ¹H NMR (400 MHz, DMSO-d₆) δ 10.44 (s, 1H), 9.05 (s, 1H), 8.61 (br s, 3H), 8.48-8.41 (m, 1H), 8.28 (d, J=8.4 Hz, 2H), 7.89 (d, J=8.4 Hz, 3H), 7.39 (d, J=7.7 Hz, 1H), 7.33-7.23 (m, 1H), 4.09 (br d, J=5.4 Hz, 2H), 3.96 (br d, J=10.1 Hz, 2H), 3.41-3.30 (m, 1H), 3.27-3.16 (m, 1H), 3.15-3.01 (m, 2H), 1.98 (br d, J=14.3 Hz, 1H), 1.91-1.80 (m, 2H), 1.73 (br d, J=14.2 Hz, 1H), 1.77-1.69 (m, 1H), 1.67-1.55 (m, 2H), 1.49 (br d. J=12.2 Hz, 6H), 1.12 (s, 3H). ESI [M+H]=584.3.

### Preparation of Compound J-2

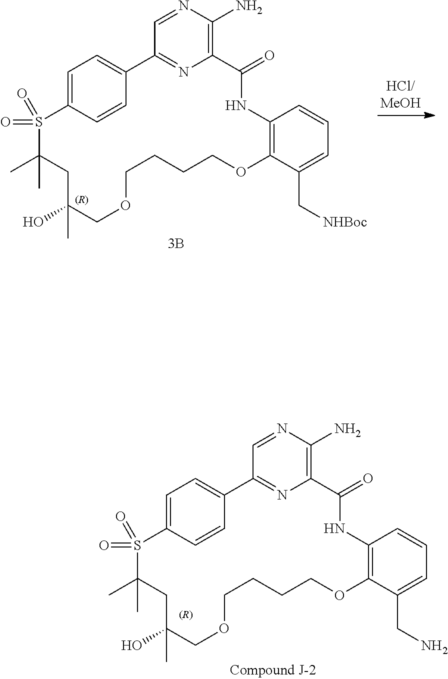

A solution of tert-butyl N-[[(32R)-28-amino-32-hydroxy-31,31,32-trimethyl-29,47,47-trioxo-44,45-dioxa-47thia-35,36,37-triazatetracyclohentriaconta-6(8),7(23),9(21),10(12),11(22),13(35),24(26),25(36),27-nonaen-23-yl]methyl]carbamate (50 mg, 73.12 μmol, 1.0 eq.) in HCl/MeOH (1 mL; 4M) was stirred at 20° C. for 0.5 hour. The reaction mixture was concentrated and the residue was purified by prep-HPLC (column: Phenomenex Luna C18 150*30 mm*5 μm; mobile phase: [water(0.04% HCl)-ACN]; B %: 10%-45%, 10 min) to give (28R)-25-amino-20-(aminomethyl)-28-hydroxy-27,27,28-trimethyl-40,40-dioxo-38,39-dioxa-40thia-31,32,33-triazatetracyclohentriaconta-3(5),4(20),6(18),7(9),8(19),10(31),21(23),22(32),24-nonaen-26-one (24 mg, 40.04 μmol, 54.76% yield, 97.382% purity) as a yellow solid. ¹H NMR (400 MHz, DMSO-d₆) δ 10.45 (s, 1H), 9.09-9.00 (m, 1H), 8.56-8.40 (m, 4H), 8.34-8.21 (m, 2H), 7.97 (br s, 4H), 7.39-7.23 (m, 2H), 4.15-4.04 (m, 2H), 4.02-3.89 (m, 2H), 3.41-3.31 (m, 1H), 3.24-3.17 (m, 1H), 3.13-2.99 (m, 2H), 2.02-1.93 (m, 1H), 1.91-1.82 (m, 2H), 1.77-1.68 (m, 1H), 1.65-1.55 (m, 2H), 1.54-1.44 (m, 6H), 1.18-1.04 (m, 3H). ESI [M+H]=584.3.

### Example 20: Assays

### A. Cell Seeding

Cells are seeded for the purpose of a specific assay or series of assays. Cell lines differ in surface area per cell and thus different numbers of cells are needed for different cell lines. The numbers of cells needed per cm² for each specific assay and cell line are indicated in Table 1.

TABLE 1
Seeding Densities by Cell Line and Assay

| | | Seeding Densities | | |
|---|---|---|---|---|
| | | Standard | Per Well (Calculated) | |
| Assay | Cell Line | Density | 6-well (k) | 96-well |
| Atrize ™ | Jurkat E6.1 | 1.5 × 10⁶/mL | NA | 150k |
| Cell Cycle | Jurkat Bcl/xl | 1.5 × 10⁶/mL | NA | 150k |
| WB | HCT116 Bcl/xl | 80 k/cm² | 800 | NA |
| | OVCAR3 | 80 k/cm² | 800 | NA |
| | OVCAR8 | 40 k/cm² | 400 | NA |
| | SKOV3 | 40 k/cm² | 400 | NA |
| | 22RV1 | 80 k/cm² | 800 | NA |
| Doubling | HCT116Bcl/xl | 32 k/cm² | 320 | NA |
| | OVCAR3 | 15 k/cm² | 150 | NA |
| | OVCAR8 | 20 k/cm² | 200 | NA |
| | SKOV3 | 20 k/cm² | 200 | NA |
| | 22RV1 | 20 k/cm² | 200 | NA |

Cells to be seeded will be two or three passages after thawing.

1. Prewarm cell media in a 37° C. water bath and 0.05% or 0.25% trypsin on bench top.

2. Examine cells to be used for seeding under 10× and 40× brightfield magnification for healthy appearance and level of confluency. For seeding, cells should be at approximately 80% so that cells are in the exponential growth phase.

3. Aspirate cell media from the flask and wash twice with 15 mL (for 75 cm² flask) or 10 mL (for 10-cm cell culture dish) of 1×PBS.

4. Add 1.5 mL of 0.05% or 0.25% trypsin media and tilt the flask/dish to make sure all the cells are exposed to the trypsin media. Place the flask/dish back in the incubator for 5 minutes.

5. When cells are dissociated from the dish surface, add prewarmed cell culture media equal to 3× the volume of trypsin previously added to deactivate the trypsin (4.5 mL). Wash the cells off the surface via gentle pipetting. The total volume of the cell suspension is now 6 mL.

6. Combine cell suspensions from all plates (if multiple plates are being used) into a 50-mL centrifuge tube.

7. Withdraw 40 μL of cell suspension and add to 40 μL of Trypan Blue dye in a 1.5-mL Eppendorf tube.

8. Add 10 μL of the cell suspension/dye mixture to each side of a cell counting slide and perform a count of the cells in an automated cell counter.

9. Record the number of live cells per side of the slide. If the counts are above 95% viability and within ˜10% of each other, average the counts to arrive at a total cell number. If the counts do not meet these criteria, repeat this step. If the counts fail again, repeat step 8. Discard remaining cell suspension/dye mixture.

10. Calculate the total number of cells in the cell suspension:

$$Total cells = ( Cells per mL from Step 9 ) * ( Total volume of cell suspension in mL )$$

11. Calculate the number of cells needed for the experiment:

$$Number of cells needed = ( Number of wells to be seeded + 2 ) * seeding density per well from Table 1$$

12. Calculate the volume of cell suspension needed for the experiment:

$$\frac{Number of cells needed}{Cell density from Step 9} = ml of cell suspension needed$$

13. Gently pellet the required volume of cell suspension from step 12 in a 50-mL centrifuge tube (1000 rpm for 5 minutes).

14. Calculate the volume of media required for resuspension of the cell pellet:

$$m L of media = number of cell suspension needed$$

15. Remove the supernatant from pelleted cells and resuspend in that calculated volume of prewarmed cell media.

16. Add 1 mL fresh prewarmed cell media to each well to be seeded in the appropriate number of sterile, labeled 6-well plates. Two 6-well plates are required for the standard exposure regimen for one compound.

17. Add 1 mL of cell resuspension to each well. Gently swirl or pipette the tube containing the cell suspension between wells to avoid cells accumulating at the bottom of the tube. Gently swirl plates using a figure 8 motion to ensure equal distribution of the cells throughout the well.

18. Re-plate the remaining cell suspension for future use at an appropriate density.

19. Compound exposure can be performed immediately for the Atrize™ Assay and the cell doubling assay; for all other assays the cells are allowed 24 hours in the incubator before compound exposure.

20. Place the plate(s) in the incubator at 37° C. with 5% CO₂.

### B. Cell Doubling Assay—Rangefinder

### (i) Cell Seeding and Treatment: Day 0

Prewarm cell media in a 37° C. water bath and 0.05% trypsin on bench top.

Compound stocks are stored at a concentration of 1 mM. Lower-potency compounds (i.e., administered at >100 nM) need not be diluted further and are used at the stored stock concentration. For high-potency compounds (i.e., administered at <100 nM), dilute the compound 1:10 in DMSO [90 μL DMSO+10 μL 1 mM compound stock] in an Eppendorf tube. The concentration is now 100 μM.

Divide the high potency diluted compound into five 20-μL compound working aliquots. Store aliquots at −20° C. Use a fresh aliquot when cells are counted and replated on subsequent days. Dilutions are 2× of the final concentration.

TABLE 2
Compound Dilutions

| | Final/ | | | | |
|---|---|---|---|---|---|
| | Dosage | | Vol Cell | | |
| | Conc. | | Media | | Vol Compound |
| Article | (nM) | 2× | (mL) | DMSO | Dilution |
| NT | n/a | n/a | 4 | Match* | n/a |
| Compound | x | 2x | 8 | n/a | as calculated |
| | 0.5x | x | 4 | | 4 mL of above + |
| | | | | | 4 mL cell media |
| | 0.25x | 0.5x | 4 | | 4 mL of above + |
| | | | | | 4 mL cell media |
| | 0.125x | 0.25x | 4 | | 4 mL of above + |
| | | | | | 4 mL cell media |
| | 0.0625x | 0.125x | 4 | | 4 mL of above + |
| | | | | | 4 mL cell media |

n/a, not applicable;
*match highest μL values calculated for dosage solutions

Use the following equations (based on CC1VV1=CC2VV2) to determine the volume of compound stock or compound working aliquot to add to the dosage media.

Volume of 1 mM compound stock to add to 8 mL media of highest concentration:

$$μL = \frac{[ 2 \times final conc in nM ] * 8}{1000} = ( final conc in nM ) * 0.016$$

Volume of 100 μM compound working aliquot to add to 8 mL media of highest concentration:

$$μ L = \frac{[ 2 \times final conc in nM * 8}{100} = ( final conc in nM ) * 0.16$$

To conduct the Range-finding Assay for one drug compound (agent), 18 wells (three 6-well plates) are required, as the study is conducted in three identical replicates. Add 1 mL of the appropriate dosage media to each well and place plates in the incubator.

Aspirate cell media from the dish(es)/flask and wash twice with 5 mL of 1× PBS. Add 1.5 mL of 0.05% trypsin media and tilt the dish/flask to make sure all the cells are exposed to the trypsin media. Place the flask/dish back in the incubator for 5 minutes.

When cells are dissociated from the dish surface, add prewarmed cell culture media equal to 3× the volume of trypsin previously added to deactivate the trypsin (4.5 mL). Wash the cells off the surface via gentle pipetting. The total volume of the cell suspension is now 6 mL (per dish/flask). Combine the suspension from all dishes if multiple dishes are being used.

Centrifuge 1 mL of Trypan Blue at maximum speed for 5 minutes. Move the supernatant to an Eppendorf tube for use in the biosafety cabinet. Withdraw 20 μL of cell suspension and add to 20 μL of Trypan Blue dye in a 1.0-mL Eppendorf tube. Add 10 μL of the cell suspension/dye mixture to each side of a cell counting slide and perform a count of the cells in an automated cell counter. Count twice (two samples). Record the number of live cells per side of the slide. If the counts are above 90% viability and within ˜10% of each other, average the counts. If the counts do not meet these criteria, repeat this step. Prepare cells according to the following numbered steps:

1. Calculate the total number of cells in the cell suspension:

$$Total cells = ( Cells per mL ) * ( Total volume of cell suspension in mL )$$

2. Calculate the number of cells needed for the experiment:

$$Number of cells neeeded = ( Number of wells to be seeded + 2 ) * ( seeding density per well from Table 3 )$$

TABLE 3
Seeding Densities by Cell Line and Assay

Seeding Densities

| | Standard | Per Well |
|---|---|---|
| | Density | (Calculated) |
| Cell Line | (k/cm²) | [6-well] |
| HCT116Bcl/xl | 32 | 320k |
| OVCAR3 | 15 | 150k |
| OVCAR8 | 20 | 200k |
| SKOV3 | 20 | 200k |
| 22RV1 | 32 | 320k |

3. Calculate the volume of cell suspension needed for the experiment:

$$\frac{Number of cells neeeded}{Average cell density from counting slide} = mL of cell suspension needed$$

4. Pellet the required volume of cell suspension from Step 3 in a 50-mL centrifuge tube (1000 rpm for 5 minutes).

5. Calculate the volume of media required for resuspension of the cell pellet:

$$mL of media = number of wells to be seeded + 2$$

6. Remove the supernatant from pelleted cells and resuspend in volume of prewarmed cell media calculated in Step 5 in a 50-mL centrifuge tube.

Gently swirl the cell suspension tube to distribute the cells within the media. For Day 0 only, add 1 mL of cell resuspension to each of the 18 wells. Note that on Days 2 and 4, the volume of cell suspension (and additional fresh media) added to wells is calculated and will vary. Place the plates in the incubator.

### (ii) Count and Replate: (Days 2 and 4)

Prewarm cell media in a 37° C. water bath and 0.05% or 0.25% trypsin on bench top. Prepare dosage solutions as described for Day 0. Aspirate cell media from the wells and wash once with 2 mL of 1×PBS. Add 250 μL of 0.05% trypsin media and tilt the dish to make sure all the cells are exposed to the trypsin media. Place the flask/dish back in the incubator for 5 minutes.

When cells are dissociated from the dish surface, add prewarmed cell culture media equal to 3× the volume of trypsin previously added to deactivate the trypsin (750 μL). Wash the cells off the surface via gentle pipetting. The total volume of the cell suspension is now 1 mL (per well). Transfer the contents of each well to the appropriate 1.5 mL tube.

For each tube, withdraw 30 μL of cell suspension and add to 30 μL of Trypan Blue dye in the associated labeled 1.0-mL Eppendorf tube. Add 10 μL of the cell suspension/dye mixture to one side of a cell counting slide and perform a count of the cells in an automated cell counter. Repeat for each well/tube.

If there are not enough cells to match the seeding density from Table 1, back-calculate the number of cells to seed per well such that no more than 850 μL of cell suspension is placed back into the well (along with 150 μL of fresh media). Adjust the equation in the spreadsheet to reflect the actual number of cells seeded. All three replicate wells of a dosage group must receive the same number of cells.

Add dosage solutions, cell suspension, and fresh media to wells as calculated.

(iii) Count Only: (Day 6) or Count and Replate if Necessary: (Day 6 or 8)

Each well is considered a separate experiment and cell suspensions are not to be pooled at any time. Count cells in the same manner as described in section (ii). If there is significant die-off in the higher dose groups, discard cell suspensions.

### (iv) Formulae

Cell doublings on Day 2 are calculated via the following formula:

$$Doublings = Log2 ( \frac{( \frac{Seeding Density}{{10}^{4}} )}{CellCount} )$$

* where CellCount is the average cell count across three replicates for Day 2.

Cell doublings on subsequent days are calculated as follows:

$$Doublings = Log2 ( \frac{( \frac{Seeding Density}{{10}^{4}} )}{CellCount} ) + PrevCount$$

* where CellCount is the average cell count across three replicates for the day in question, and PrevCount is the average cell count across three replicates for the previous counting day.

### C. Cell Doubling Assay—Two Agents

### (i) Cell Seeding and Treatment: Day 0

Compound stocks are stored at a concentration of 1 mM. Lower-potency compounds (i.e., administered at >100 nM) need not be diluted further and are used at the stored stock concentration. For high-potency compounds (i.e., administered at <100 nM), dilute the compound 1:10 in DMSO [90 μL DMSO+10 μL 1 mM compound stock] in an Eppendorf tube. The concentration is now 100 μM.

Divide the high potency diluted compound into five 20-μL compound working aliquots. Store aliquots at −20° C. Use a fresh aliquot when cells are counted and replated on subsequent days. Dilute each compound into 4 mL cell media such that the compound is at 4× of the final concentration to be administered to the cells; the administered 1 mL of diluted compound in cell media will constitute 25% of the volume of each well. See Table 3 for well final contents.

Add compound or DMSO vehicle and cell media as shown in Table 4 to three 15-mL centrifuge tubes with the dosage levels.

Prewarm cell media in a 37° C. water bath and 0.05% trypsin on bench top.

TABLE 4
Compound Dilutions

| | Target | | Final Contents | | |
|---|---|---|---|---|---|
| | Conc. | | Cell | | Vol Compound |
| Article | (nM) | 4× | Media | DMSO | Dilution |
| NT | n/a | n/a | 4 mL | Match* | n/a |
| Compound 1 | x | 4x | | n/a | As calculated |
| Compound 2 | x | | | n/a | As calculated |

n/a, not applicable;
*match higher of two μL values calculated for dosage solutions

Use the following equations (based on CC1VV=CC2VV2) to determine the volume of compound stock or compound working aliquot to add to the dosage media.

Volume of 1 mM compound stock to add to 4 mL media:

$$μ L = \frac{[ 4 \times final conc in nM ] * 4}{1000} = ( final conc in nM ) * 0.016$$

Volume of 100 μM compound working aliquot to add to 4 mL media:

$$μ L = \frac{[ 4 \times final conc in nM ] * 4}{100} = ( final conc in nM ) * 0.16$$

To conduct the assay for two drug compounds (agents), 12 wells (two 6-well plates) are required, as the study is conducted in three identical replicates. Add dosage media plus fresh media to wells according to Table 5, then put plates in incubator.

TABLE 5
Dosage and Fresh media added to wells, Day 0

| | | | |
|---|---|---|---|
| Plate 1 | 500 μL NT | 500 μL NT | 500 μL NT |
| | solution + 500 μL | solution + 500 μL | solution + 500 μL |
| | fresh media* | fresh media | fresh media |
| | 500 μL Compound 1 | 500 μL Compound 1 | 500 μL Compound 1 |
| | solution + 500 μL | solution + 500 μL | solution + 500 μL |
| | fresh media | fresh media | fresh media |
| Plate 2 | 500 μL Compound 2 | 500 μL Compound 2 | 500 μL Compound 2 |
| | solution + 500 μL | solution + 500 μL | solution + 500 μL |
| | fresh media | fresh media | fresh media |
| | 500 μL Compound 1 | 500 μL Compound 1 | 500 μL Compound 1 |
| | solution + 500 μL | solution + 500 μL | solution + 500 μL |
| | Compound 2 | Compound 2 | Compound 2 |
| | solution | solution | solution |

Aspirate cell media from the dish(es)/flask and wash twice with 5 mL of 1× PBS. Add 1.5 mL of 0.05% trypsin media and tilt the dish/flask to make sure all the cells are exposed to the trypsin media. Place the flask/dish back in the incubator for 5 minutes.

When cells are dissociated from the dish surface, add prewarmed cell culture media equal to 3× the volume of trypsin previously added to deactivate the trypsin (4.5 mL). Wash the cells off the surface via gentle pipetting. The total volume of the cell suspension is now 6 mL (per dish/flask). Combine the suspension from all dishes if multiple dishes are being used.

Centrifuge 1 mL of Trypan Blue at maximum speed for 5 minutes. Move the supernatant to an Eppendorf tube for use in the biosafety cabinet. Withdraw 20 μL of cell suspension and add to 20 μL of Trypan Blue dye in a 1.0-mL Eppendorf tube.

Add 10 μL of the cell suspension/dye mixture to each side of a cell counting slide and perform a count of the cells in an automated cell counter. Count twice (two samples). Record the number of live cells per side of the slide. If the counts are above 90% viability and within ˜10% of each other, average the counts. If the counts do not meet these criteria, repeat this step. Discard remaining cell suspension/dye mixture. Prepare cells according to the following numbered steps:

1. Calculate the total number of cells in the cell suspension:

$$Total cells = ( Cells per mL ) * ( Total volume of cell suspension in mL )$$

2. Calculate the number of cells needed for the experiment according to Table 3.

$$Number of cells neeeded = ( Number of wells to be seeded + 2 ) * ( seeding density per well from Table 3 )$$

3. Calculate the volume of cell suspension needed for the experiment:

$$\frac{Number of cells neeeded}{Average cell density from counting slide} = mL of cell suspension needed$$

4. Pellet the required volume of cell suspension from Step 3 in a 50-mL centrifuge tube (1000 rpm for 5 minutes).

5. Calculate the volume of media required for resuspension of the cell pellet:

$$mL of media = number of wells to be seeded + 2$$

6. Remove the supernatant from pelleted cells and resuspend in volume of prewarmed cell media calculated in Step 5 in a 50-mL centrifuge tube.

Gently swirl the cell suspension tube to distribute the cells within the media. For Day 0 only, add 1 mL of cell resuspension to each of the 12 wells. On Days 2 and 4, the volume of cell suspension (and additional fresh media) added to wells is calculated and will vary from the values in Table 3. Place the plates in the incubator.

(ii) Count and Replate: (Days 2 and 4, optional: Day 6)

Prewarm cell media in a 37° C. water bath and 0.05% or 0.25% trypsin. Each well is considered a separate experiment and cell suspensions are not to be pooled at any time. Aspirate cell media from the wells and wash once with 2 mL of 1×PBS. Add 250 μL of 0.05% trypsin media and tilt the dish to make sure all the cells are exposed to the trypsin media. Place the flask/dish back in the incubator for 5 minutes.

When cells are dissociated from the dish surface, add prewarmed cell culture media equal to 3× the volume of trypsin previously added to deactivate the trypsin (750 μL). Wash the cells off the surface via gentle pipetting. The total volume of the cell suspension is now 1 mL (per well). Transfer the contents of each well to the appropriate labeled 1.5 mL tube.

For each tube, withdraw 30 μL of cell suspension and add to 30 μL of Trypan Blue dye in the associated labeled 1.0-mL Eppendorf tube. Add 10 μL of the cell suspension/dye mixture to one side of a cell counting slide and perform a count of the cells in an automated cell counter. Repeat for each well/tube.

If there are not enough cells to match the seeding density from Table 1, back-calculate the number of cells to seed per well such that no more than 850 μL of cell suspension is placed back into the well (along with 150 μL of fresh media). Adjust the equation in the spreadsheet to reflect the actual number of cells seeded. All three replicate wells of a dosage group must receive the same number of cells; calculate accordingly. Add dosage solutions, cell suspension, and fresh media to wells as calculated.

(iii) Count Only or Count and Replate if Necessary: (Day 6 or 8)

Each well is considered a separate experiment and cell suspensions are not to be pooled at any time. Count cells in the same manner as described in section (ii). These results will be entered into the spreadsheet prepared for the experiment. Enter the counts into the doubling curve in the spreadsheet; if there is not significant decrease in doubling of the combination (Compound 1+Compound 2) wells, place tubes with cell suspensions into the incubator and prepare dosage solutions as described for Day 0. Then follow the rest of the procedure for Days 2 and 4.) If there is significant die-off in the combination group, discard cell suspensions.

### (iv) Formulae

Cell doublings on Day 2 are calculated via the following formula:

$$Doublings = Log2 ( \frac{( \frac{Seeding Density}{{10}^{4}} )}{CellCount} )$$

where CellCount is the average cell count across three replicates for Day 2.

Cell doublings on subsequent days are calculated as follows:

$$Doublings = Log2 ( \frac{( \frac{Seeding Density}{{10}^{4}} )}{CellCount} ) + PrevCount$$

where CellCount is the average cell count across three replicates for the day in question, and PrevCount is the average cell count across three replicates for the previous counting day.

### D. Protein Quantification

Prepare and aliquot BSA standards.

1. Remove prepared BSA standard aliquots from freezer and allow to equilibrate to room temperature. Vortex each aliquot briefly.

2. Remove harvested treated cell samples from freezer and allow to equilibrate to room temperature. Vortex each sample briefly.

3. Add standards and samples to a 96-well flat-bottomed plate as described in the following steps.

4. Add the BSA standards.
Add 25 μL of the first BSA standard (1500 μg/mL) to each of wells A1 and B1.
Add 25 μL of the first BSA standard (1000 μg/mL) to each of wells A2 and B2.
Add 25 μL of the first BSA standard (750 μg/mL) to each of wells A3 and B3.
Add 25 μL of the first BSA standard (500 μg/mL) to each of wells A4 and B4.
Add 25 μL of the first BSA standard (250 μg/mL) to each of wells A5 and B5.
Add 25 μL of the first BSA standard (125 μg/mL) to each of wells A6 and B6.
Add 25 μL of the first BSA standard (25 μg/mL) to each of wells A7 and B7
Add 25 μL of the first BSA standard (0 μg/mL[Blank]) to each of wells A8 and B8.

5. Add the samples from harvested cells. (Row D is a duplicate of Row C). The following steps list the dose levels that are indicated in the standard dosing regimen. If the dosing regimen used differs, adjust accordingly. Note that untreated. APH+ samples bracket the treated dose range.
Add 25 μL of the first sample (NT) to each of wells C1 and D1.
Add 25 μL of the first sample (APH+) to each of wells C2 and D2.
Add 25 μL of the first sample (100 nM) to each of wells C3 and D3.
Add 25 μL of the first sample (30 nM) to each of wells C4 and D4.
Add 25 μL of the first sample (10 nM) to each of wells C5 and D5.
Add 25 μL of the first sample (3 nM) to each of wells C6 and D6.
Add 25 μL of the first sample (1 nM) to each of wells C7 and D7
Add 25 μL of the first sample (300 pM) to each of wells C8 and D8.
Add 25 μL of the first sample (100 pM) to each of wells C9 and D9.
Add 25 μL of the first sample (30 pM) to each of wells C10 and D10.
Add 25 μL of the first sample (10 pM) to each of wells C11 and D11
Add 25 μL of the first sample (APH+) to each of wells C12 and D12.

6. Calculate the volume of Reagent A from the kit required for the number of wells times 200 μL plus 10%, and add that amount to a 15-mL centrifuge tube.

7. Add 1/50^{th} (i.e., multiply by 0.02) of that volume of Reagent B from the kit to the centrifuge tube. Mix well by inversion.

8. Place the reagent mixture in a reagent well.

9. Transfer 200 μL of the reagent mix to each well of the standards and samples in the 96-well plate. Pipette up and down to mix.

10. Cover the plate.

11. Place the plate in the incubator for 30 minutes.

12. Remove from the incubator and allow to equilibrate to room temperature in a biosafety cabinet.

13. Analyze results using the microplate reader.

### E. Treatment of Cells for IC₅₀ Assay by Western Blot

The compound is stored as a 1 mM stock solution prepared in accordance with the Receipt of Compound SOP. The stock solution is diluted in cell media in two separate 1:10 dilution series. The series of dilutions of compound for treatment are listed in Tables 6 and 6.

TABLE 6
Dilution Series 1 (1 μM-10 pM)

| | | Volume of |
|---|---|---|
| | | (Prewarmed) |
| Concentration | Volume of 1 mM Compound | Cell Media |
| of Compound | Stock or Dilution | (mL) |

| | | | |
|---|---|---|---|
| 1 | μM* | 2.5 μL of 1 mM stock | 2.5 |
| 100 | nM | 250 μL of 1 μM dilution | 2.25 |
| 10 | nM | 250 μL of 100 nM dilution | 2.25 |
| 1 | nM | 250 μL of 10 nM dilution | 2.25 |
| 100 | pM | 250 μL of 1 nM dilution | 2.25 |
| 10 | pM | 250 μL of 0.1 nM dilution | 2.25 |

*used to facilitate dilution and not used for exposure to cells.

TABLE 7
Dilution Series 2 (3 μM-30 pM)

| | | Volume of |
|---|---|---|
| | Volume of 1 mM | (Prewarmed) |
| Concentration | Compound Stock | Cell Media |
| of Compound | or Mixture | (mL) |

| | | | |
|---|---|---|---|
| 3 | μM* | 7.5 μL of 1 mM stock | 2.5 |
| 300 | nM* | 250 μL of 3 μM | 2.25 |
| 30 | nM | 250 μL of 300 nM | 2.25 |
| 3 | nM | 250 μL of 30 nM | 2.25 |
| 300 | pM | 250 μL of 3 nM | 2.25 |
| 30 | pM | 250 μL of 0.3 nM | 2.25 |

*used to facilitate dilution and not used for exposure to cells.

1. Arrange twelve 15-mL centrifuge tubes in a rack in two series and label them in accordance with Dilutions Series 1 and Dilution Series 2 shown in Table 1 and Table 2, respectively.

2. Place 2.5 mL of prewarmed cell media in each of the 100 nM tube (Series 1) and 300 nM tube (Series 2). Place 2.25 mL of prewarmed cell media in the remaining tubes.

3. Add 7.5 μL of 1 mM compound stock solution in the 3 μM tube. Mix well.

4. Add 2.5 μL of 1 mM compound stock solution in the 1 μM tube. Mix well.

5. For each series (separately), remove 250 μL from the leftmost tube and transfer to the next tube to the right. Repeat for each tube until the series is complete.

6. Set aside the 1 μM tube, the 3 μM tube, and the 300 nM tube for discard.

7. The cells to be treated will be in 6-well plates labeled by cell line. Remove the cells from the incubator and label the wells with the dosages to be administered.

8. Arrange the dilution tubes in the rack in a manner that reflects the 6-well plate arrangement so as to avoid confusion when administering the dosage solutions to the appropriate wells.

9. Aspirate the three wells in the top row of the first plate (labeled NT, APH+, and APH+) and replace with 2 mL fresh media (without drug compound).

10. Aspirate the three wells in the bottom row of the first plate and replace each well with 2 mL of solution from the appropriate tube.

11. Repeat step 10 for the next row of wells (top row second plate). and then for the final row of wells (bottom row second plate).

12. Place both plates into the incubator for 30 minutes.

13. After 30 minutes have elapsed, remove the plates from the incubator.

14. Acquire stock solution of APH (5 mM) and add 2 μL of the APH stock solution to the center of each well except the well labeled NT.

15. Swirl the plates carefully and gently to diffuse the APH throughout each well.

16. Place the plates back in the incubator for 4 hours, at which point the cells will be harvested for analysis.

17. Twenty minutes before harvesting (or 3 hours and 40 minutes after administration of APH), turn on a heat block suitable to heat Eppendorf tubes. Set the temperature to 95° C.

18. Set up a number of Eppendorf tubes equal to the number of wells treated (i.e., twelve per two-plate drug administration), and label each tube with cell line, compound administered, dosage level, and date. Leave the tubes open.

19. Add 200 mL DI water to a beaker.

20. After 4 hours, remove treated plates from incubator.

21. Aspirate media from all wells.

22. Wash each well with 2 mL 1×PBS and aspirate.

23. Sterilize vacuum line with 70% ethanol by spraying some into the nozzle while the vacuum is engaged. Shut off vacuum.

24. Add Laemmli buffer to each well. For HCT116 Bcl/xl, OVCAR-3, and 22RV1 cells, add 300 μL per well; for OVCAR-8 and SKOV-3 cells, add 250 μL per well.

25. Scrape the first well thoroughly. Place the scraper into the DI water between uses.

26. Transfer the lysed cell debris mixture to the first tube carefully. The mixture will be viscous. Use a 1000-μL pipettor. Close the tube.

27. Repeat steps 25 and 26 for each well.

28. When all wells have been harvested, place all the tubes into the heat block. Place a weighted, heat-resistant object on top of the tubes to keep them closed.

29. After 5 minutes, remove the tubes from the heat block and turn it off. Vortex each tube for 10 seconds. Be cautious of tubes popping open.

30. Allow the tubes to come to room temperature and proceed to further analysis or store frozen at −20° C.

### F. Results

Compound X was prepared as described in U.S. Pat. No. 9,663,535, which is hereby incorporated by reference.

VE822 and AZD 6738 are ATR inhibitors are available in the art.

TABLE 8

| | | | IC₅₀ [PALD] nM | | | | |
|---|---|---|---|---|---|---|---|
| | | | (Percent Activity at Lowest Dose tested) | | | | |
| Racemic | | MEC | HCT | | | | |
| Compound | Enantiomer | (+APH) | Bcl/xl | OVCAR-3 | OVCAR-8 | SKOV-3 | 22RV1 |
| X | NA | 0.3 | 4.8 | — | 52.7 | 8.43 | 23.98 |
| | | | (111%) | | (123%) | (87%) | (106%) |
| VE822 | NA | 0.3 | 62.1 | 11.46 | 84.6 | 9.5 | 84.9 |
| | | | (105%) | (79%) | (89%) | (55%) | (86%) |
| AZD | NA | 0.3 | 57.15 | 91.78 | 86.6 | 106.6 | 275.3 |
| 6738 | | | | (190%) | (116%) | (105%) | (165%) |
| A | — | 0.02 | 3.49 | 2.19 | 1.68 | 2.39 | 8.28 |
| | | | (74%) | (111%) | (71%) | (88%) | (73%) |
| | A-1 | 0.02 | 2.68 | 4.09 | 2.16 | 2.85 | 4.69 |
| | | | (110%) | (116%) | (91%) | (81%) | (121%) |
| | A-2 | 0.02 | 2.29 | 0.753 | 2.95 | 2.71 | 3.44 |
| | | | (91%) | (101%) | (82%) | (110%) | (107%) |
| B | — | 0.04 | 4.63 | 2.15 | 4.47 | 2.62 | 2.99 |
| | | | (115%) | (121%) | (94%) | (106%) | (141%) |
| | B-1 | 0.04 | 4.61 | 7.43 | 7.06 | 1.57 | 4.953 |
| | | | (93%) | (105%) | (132%) | (106%) | (98%) |
| | B-2 | 0.02 | 3.0 | 3.12 | 2.62 | 0.97 | 1.89 |
| | | | (81%) | (94%) | (115%) | (115%) | (101%) |
| C | — | 0.01 | 2.06 | 3.85 | 6.8 | 1.085 | 4.32 |
| | | | (65%) | (158%) | (73%) | (100%) | (90%) |
| | C-1 | 0.01 | 4.53 | 1.02 | 3.63 | — | 3.5 |
| | | | (72%) | (115%) | (83%) | | (107%) |
| | C-2 | 0.01 | 5.41 | 11.5 | 2.47 | 3.2 | 2.03 |
| | | | (71%) | (95%) | (100%) | (95%) | (92%) |
| D | — | 0.04 | 1.27 | — | 4.69 | 2.63 | 1.97 |
| | | | (73%) | | (92%) | (67%) | (81%) |
| E | — | 0.08 | 6.5 | — | 6.47 | — | 3.28 |
| | | | (91%) | | (139%) | | (72%) |
| F | — | 0.156 | 14.08 | 8.23 | 31.46 | — | 10.75 |
| | | | (104%) | (86%) | (81%) | | (96%) |
| G | — | 0.08 | 12.51 | 4.97 | — | 2.64 | 5.09 |
| | | | (109%) | (83%) | | (97%) | (104%) |
| | G-1 | 0.04 | 7.77 | — | 4.97 | | 7.02 |
| | | | (115%) | | (85%) | | (111%) |
| | G-2 | 0.04 | 7.90 | — | — | 4.94 | 8.57 |
| | | | (83%) | | | (92%) | (86%) |
| H | — | 0.02 | 3.7 | — | 4.42 | 1.3 | 3.81 |
| | | | (106%) | | (68%) | (104%) | (94%) |
| | H-1 | 0.04 | 4.46 | — | — | — | 7.84 |
| | | | (89%) | | | | (71%) |
| | H-2 | 0.02 | 6.43 | — | — | — | — |
| | | | (89%) | | | | |

| | | Ave. IC₅₀ | |
|---|---|---|---|
| Racemic | | across cell | |
| Compound | Enantiomer | lines (nM) | SD |
| X | NA | 20.63 | 17.28 |
| VE822 | NA | 50.0 | — |
| AZD 6738 | NA | 123.49 | |
| A | — | 3.61 | 2.4 |
| | A-1 | 3.29 | 0.9 |
| | A-2 | 2.42 | 0.9 |
| B | — | 3.37 | 1.0 |
| | B-1 | 5.12 | 2.1 |
| | B-2 | 2.32 | 0.8 |
| C | — | 3.6 | 2.0 |
| | C-1 | 2.7 | 1.5 |
| | C-2 | 4.92 | 3.5 |
| D | — | 2.6 | 1.3 |
| E | — | — | — |
| F | — | — | — |
| G | — | — | — |
| | G-1 | — | — |
| | G-2 | — | — |
| H | — | 3.3 | 1.2 |
| | H-1 | — | — |
| | H-2 | — | — |

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A compound of Formula (I): 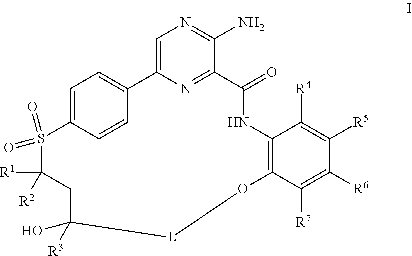
wherein:
R¹ is H, C₁₋₆alkyl, or substituted C₁₋₆alkyl;
R² is H, C₁₋₆alkyl, or substituted C₁₋₆alkyl;
or R¹ and R² are joined to form an optionally substituted C₃₋₆cycloalkyl or an optionally substituted heterocycloalkyl;
R³ is H, C₁₋₆alkyl, or substituted C₁₋₆alkyl;
R⁴, R⁵, R⁶, and R⁷ are, independently, H, C₁₋₆alkyl, or substituted C₁₋₆alkyl;
L is C₁₋₂₀alkylene, wherein one or more carbon atoms of the C₁₋₂₀alkylene are each optionally replaced with an oxygen atom;
or a pharmaceutically acceptable salt thereof.

2. The compound of claim 1, wherein R¹, R², R³, R⁴, R⁵, R⁶, and/or R⁷ is H.

3. The compound of claim 1, wherein R¹, R², R³, R⁴, R⁵, R⁶, and/or R⁷ is C₁₋₆alkyl.

4. The compound of claim 1, wherein R¹, R², R³, R⁴, R⁵, R⁶, and/or R⁷ is substituted C₁₋₆alkyl, such as substituted C₁₋₄alkyl, or such as substituted methyl, substituted ethyl, substituted propyl, substituted butyl, substituted pentyl, or substituted hexyl.

5-7. (canceled)

8. The compound of claim 1, wherein R¹ and R² are joined to form an unsubstituted or substituted C₃₋₆cycloalkyl.

9. (canceled)

10. The compound of claim 1, wherein R¹ and R² are joined to form an unsubstituted or substituted heterocycloalkyl, such as substituted or unsubstituted pyrrolidinyl, substituted or unsubstituted tetrahydrofuranyl, substituted or unsubstituted oxetanyl, substituted or unsubstituted pyranyl, substituted or unsubstituted piperidinyl, or substituted or unsubstituted azetidinyl.

11-17. (canceled)

18. The compound of claim 1, wherein R⁴, R⁵, R⁶, R⁷, and/or R⁸ is substituted with NH₂, NH(C₁₋₆alkyl), or NH(C₁₋₆alkyl)(C₁₋₆alkyl), such as NHCH₃, NHCH₂CH₃, N(CH₃)₂, or C(CH₂CH₃)₂, or such as NH₂, or such as NCH₃.

19-30. (canceled)

31. The compound of claim 1, wherein L is unsubstituted or substituted C₁₋₂₀alkyl.

32-33. (canceled)

34. The compound of claim 1, wherein L comprises one or more oxygen atoms.

35. The compound of claim 34, wherein L comprises two oxygen atoms, three oxygen atoms, four oxygen atoms, five oxygen atoms, or six oxygen atoms.

36. The compound of claim 1, that is: 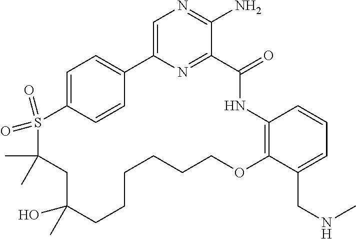
or a pharmaceutically acceptable salt thereof.

37. The compound of claim 1, that is: 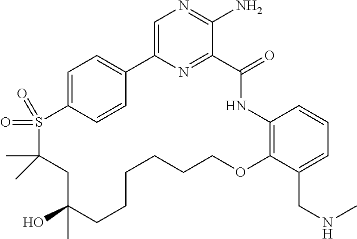 [SMILES: CNCc1cccc2c1OCCCCCC[C@@](C)(O)CC(C)(C)S(=O)(=O)c1ccc(cc1)-c1cnc(N)c(n1)C(=O)N2]
or a pharmaceutically acceptable salt thereof.

38. The compound of claim 1, that is: 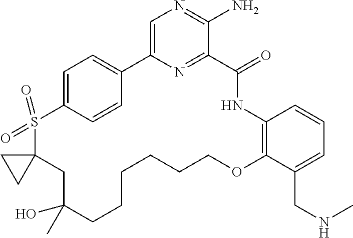 [SMILES: CNCc1cccc2c1OCCCCCCC(C)(O)CC1(CC1)S(=O)(=O)c1ccc(cc1)-c1cnc(N)c(n1)C(=O)N2]
or a pharmaceutically acceptable salt thereof.

39. The compound of claim 1, that is 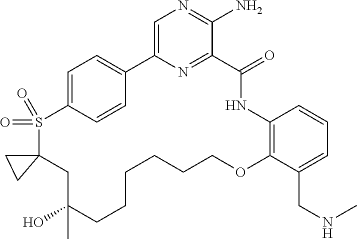 [SMILES: CNCc1cccc2c1OCCCCCC[C@](C)(O)CC1(CC1)S(=O)(=O)c1ccc(cc1)-c1cnc(N)c(n1)C(=O)N2]
or a pharmaceutically acceptable salt thereof.

40. The compound of claim 1, that is: 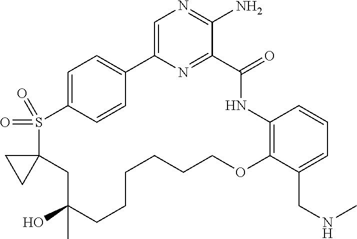 [SMILES: CNCc1cccc2c1OCCCCCC[C@@](C)(O)CC1(CC1)S(=O)(=O)c1ccc(cc1)-c1cnc(N)c(n1)C(=O)N2]
or a pharmaceutically acceptable salt thereof.

41. The compound of claim 1, that is: 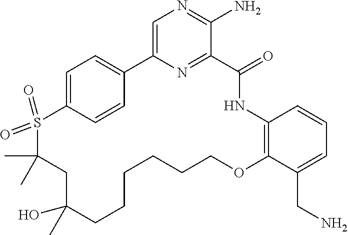 [SMILES: CC1(O)CCCCCCOc2c(CN)cccc2NC(=O)c2nc(cnc2N)-c2ccc(cc2)S(=O)(=O)C(C)(C)C1]
or a pharmaceutically acceptable salt thereof.

42. The compound of claim 1, that is: 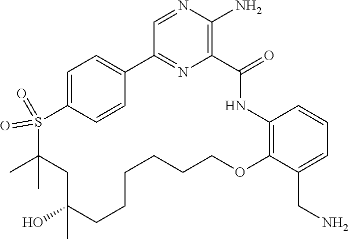 [SMILES: CC1(C)C[C@@](C)(O)CCCCCCOc2c(CN)cccc2NC(=O)c2nc(cnc2N)-c2ccc(cc2)S1(=O)=O]
or a pharmaceutically acceptable salt thereof.

43. The compound of claim 1, that is: 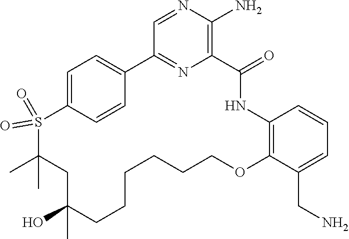 [SMILES: CC1(C)C[C@](C)(O)CCCCCCOc2c(CN)cccc2NC(=O)c2nc(cnc2N)-c2ccc(cc2)S1(=O)=O]
or a pharmaceutically acceptable salt thereof.

44. The compound of claim 1, that is: 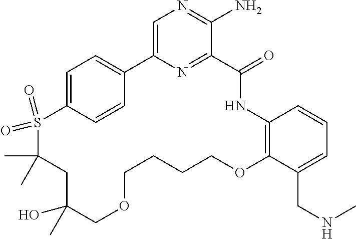
or a pharmaceutically acceptable salt thereof.

45. The compound of claim 1, that is: 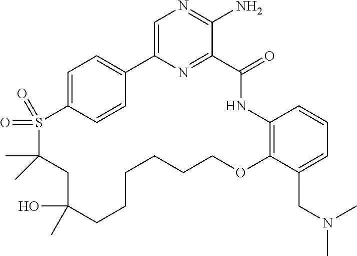
or a pharmaceutically acceptable salt thereof.

46. The compound of claim 1, that is: 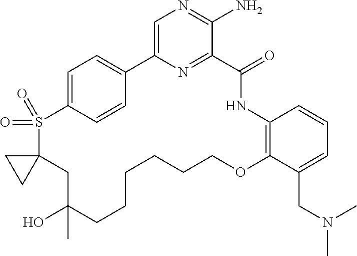 [SMILES: CN(C)Cc1cccc2c1OCCCCCCC(C)(O)CC1(CC1)S(=O)(=O)c1ccc(cc1)-c1cnc(N)c(n1)C(=O)N2]
or a pharmaceutically acceptable salt thereof.

47. The compound of claim 1, that is: 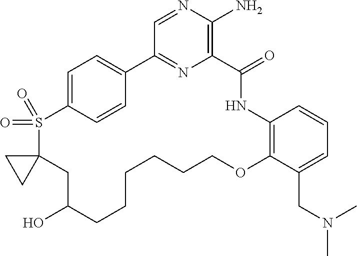 [SMILES: CN(C)Cc1cccc2c1OCCCCCCC(O)CC1(CC1)S(=O)(=O)c1ccc(cc1)-c1cnc(N)c(n1)C(=O)N2]
or a pharmaceutically acceptable salt thereof.

48. The compound of claim 1, that is: 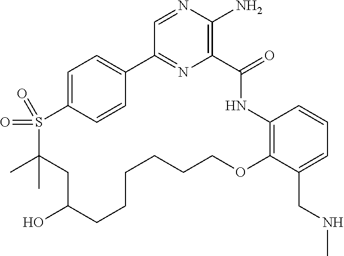 [SMILES: CNCc1cccc2c1OCCCCCCC(O)CC(C)(C)S(=O)(=O)c1ccc(cc1)-c1cnc(N)c(n1)C(=O)N2]
or a pharmaceutically acceptable salt thereof.

49. The compound of claim 1, that is: 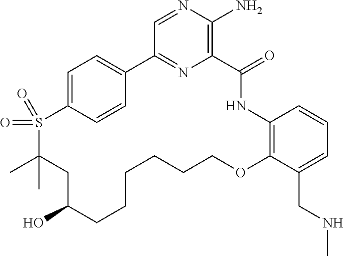 [SMILES: CNCc1cccc2c1OCCCCCC[C@@H](O)CC(C)(C)S(=O)(=O)c1ccc(cc1)-c1cnc(N)c(n1)C(=O)N2]
or a pharmaceutically acceptable salt thereof.

50. The compound of claim 1, that is: 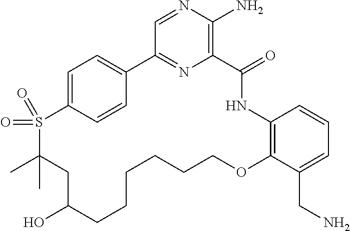 [SMILES: CC1(C)CC(O)CCCCCCOc2c(CN)cccc2NC(=O)c2nc(cnc2N)-c2ccc(cc2)S1(=O)=O]
or a pharmaceutically acceptable salt thereof.

51. The compound of claim 1, that is: 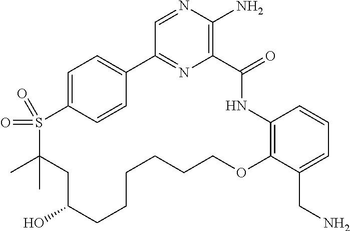 [SMILES: CC1(C)C[C@@H](O)CCCCCCOc2c(CN)cccc2NC(=O)c2nc(cnc2N)-c2ccc(cc2)S1(=O)=O]
or a pharmaceutically acceptable salt thereof.

52. The compound of claim 1, that is: 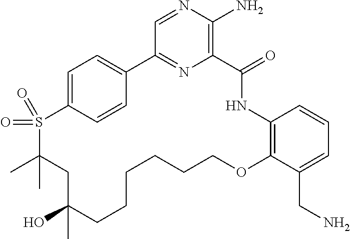 [SMILES: CC1(C)C[C@](C)(O)CCCCCCOc2c(CN)cccc2NC(=O)c2nc(cnc2N)-c2ccc(cc2)S1(=O)=O]
or a pharmaceutically acceptable salt thereof.

53. The compound of claim 1, that is: 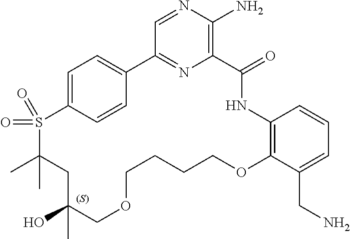
or a pharmaceutically acceptable salt thereof.

54. The compound of claim 1, that is: 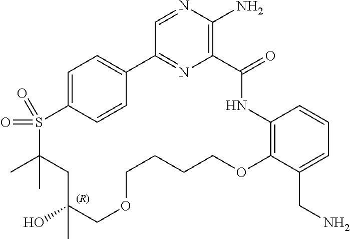
or a pharmaceutically acceptable salt thereof.

55. The compound of claim 1, wherein the salt is an HCl salt.

56. A pharmaceutical composition, comprising one or more compound of claim 1.

57. A method of treating cancer in a patient comprising administering to the patient the compound of claim 1.

58. The method of claim 57, wherein the cancer is brain cancer, breast cancer, central nervous system cancer, colorectal cancer, glioblastoma, melanoma, leukemia, liver cancer, lung cancer, lymphoma, pancreatic cancer, prostate cancer, ovarian cancer, or renal cancer.

59. The method of claim 57, wherein the cancer is ocular melanoma, desmoplastic round cell tumor, chondrosarcoma, leptomengial disease, diffuse large B-cell lymphoma, acute lymphoblastic leukemia, acute myeloid leukemia, adrenocortical carcinoma, an AIDS-related cancer, an AIDS-related lymphoma, anal or rectal cancer, appendix cancer, astrocytomas, atypical teratoid/rhabdoid tumor, basal cell carcinoma, bile duct cancer, bladder cancer, bone cancer, osteosarcoma or malignant fibrous histiocytoma, brain tumor, breast cancer, prostate cancer, bronchial tumor, Burkitt lymphoma, spinal cord tumor, carcinoid tumor, carcinoma of unknown primary, central nervous system atypical teratoid/rhabdoid tumor, leptomeningeal disease, central nervous system embryonal tumors, central nervous system lymphoma, chordoma, chronic lymphocytic leukemia, chronic myelogenous leukemia, chronic myeloproliferative disorders, colon cancer, colorectal cancer, craniopharyngioma, cutaneous T-Cell lymphoma, ependymoblastoma, ependymoma, esophageal cancer, a Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal Tumor (GIST), germ cell tumor, glioma, hairy cell leukemia, head or neck cancer, hepatocellular (liver) cancer, histiocytosis, Hodgkin's lymphoma, hypopharyngeal cancer, Kaposi sarcoma, kidney (renal) cancer, Langerhan's cell histiocytosis, laryngeal cancer, lip or oral cavity cancer, lung cancer, Non-Hodgkin's lymphoma, primary central nervous system lymphoma, Waldenstrom's macroglobulinemia (lymphoplasmacytic lymphoma), medulloblastoma, medulloepithelioma, melanoma, merkel cell carcinoma, mesothelioma, metastatic squamous neck cancer with occult primary, multiple endocrine neoplasia syndrome, mouth cancer, multiple myeloma/plasma cell neoplasm, mycosis fungoides, myelodysplastic syndromes, myelodysplastic/myeloproliferative neoplasms, multiple myeloma, myeloproliferative disorder, nasal cavity or paranasal sinus cancer, nasopharyngeal cancer, neuroblastoma, oral cancer, oropharyngeal cancer, osteosarcoma or malignant fibrous histiocytoma of bone, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, prostate cancer, rectal cancer, pelvis or ureter cancer, respiratory tract carcinoma involving the NUT gene on chromosome 15, retinoblastoma, rhabdomyosarcoma, high grade prostate cancer, medium grade prostate cancer, low grade prostate cancer, castration-resistant prostate cancer, salivary gland cancer, sarcoma, Sezary syndrome, skin cancer such as a skin carcinoma, ocular cancer, small intestine cancer, soft tissue sarcoma, squamous cell carcinoma, squamous neck cancer with occult primary, supratentorial primitive neuroectodermal tumors, T-Cell lymphoma, testicular cancer, throat cancer, thymoma or thymic carcinoma, thyroid cancer, transitional cell cancer of the renal, pelvis or ureter, cancer of unknown primary site such as carcinoma of unknown primary site, unusual cancers of childhood, urethral cancer, Wilm's tumor, or a women's cancer such as breast cancer, cervical cancer, endometrial cancer, gestational trophoblastic tumor, ovarian cancer, ovarian germ cell tumor, ovarian epithelial cancer, ovarian low malignant potential tumor, pregnancy cancer, uterine sarcoma, vaginal cancer, or vulvar cancer.

60. The method of claim 57, wherein the compound is administered orally.
